(19) United States
(12) Patent Application Publication
(10) Pub. No.: US 2025/0281629 A1
(43) Pub. Date: Sep. 11, 2025

(54) COMPOSITIONS AND METHODS FOR ANTI-PHOSPHOCHOLINE ACTIVE IMMUNOTHERAPY

(71) Applicant: Duke University, Durham, NC (US)

(72) Inventors: Joel Collier, Durham, NC (US); Elizabeth Curvino, Durham, NC (US)

(21) Appl. No.: 18/859,471

(22) PCT Filed: Apr. 25, 2023
(86) PCT No.: PCT/US2023/019876

§ 371 Date: Oct. 23, 2024

Related U.S. Application Data
(60) Provisional application No. 63/334,259, filed on Apr. 25, 2022.

(51) Int. Cl. A61K 47/64; C07K 7/06; C07K 7/08; C07K 14/00

(52) U.S. Cl. CPC A61K 47/646; C07K 7/06; C07K 7/08; C07K 14/001

(57) ABSTRACT

Embodiments are directed to a phosphorylcholine (PC)-conjugate peptide including a self-assembling peptide and at least one PC molecule. The PC-conjugate peptide may self-assemble into a nanofiber or fibril. Compositions including the PC-conjugate peptide may be used to elicit an immune response, such as stimulating B1a cells to produce antibodies. The compositions may be used to treat an inflammatory disease such as inflammatory bowel disease (IBD) or ulcerative colitis. The compositions may also be used to alter the gut microbiome.

### CROSS-REFERENCE TO RELATED APPLICATIONS

This application claims priority to U.S. Provisional Patent Application No. 63/334,259, filed Apr. 25, 2022, which is incorporated herein by reference in its entirety.

### STATEMENT REGARDING FEDERALLY SPONSORED RESEARCH

This invention was made with government support under grant R01 EB009701 awarded by the National Institutes of Health (NIH). The government has certain rights in the invention.

### FIELD

Embodiments of this invention are directed generally to biology, medicine, and immunology. Certain aspects are directed to immunogenic peptide conjugates and nanofibers and their use in inducing an immune response.

### INTRODUCTION

Inflammatory bowel disease (IBD) is an autoimmune disorder characterized by chronic inflammation in the gastrointestinal (GI) tract that afflicts nearly 7 million people worldwide. The precise etiology of IBD is not completely understood, however, it is known that a combination of decreased barrier function of the intestinal epithelium and defects in mucosal immunity contribute to a breakdown in tolerance mechanisms that result in immune responses to commensal microbiota. This produces pro-inflammatory mediators that further damage the epithelium, increase antigen exposure, and produce a positive feedback loop that leads to chronic immune-directed inflammation. The resulting unresolved intestinal damage leads to poor nutrient absorption, bleeding, and other GI symptoms that can be life-threatening. Current therapies for IBD, such as corticosteroids, only temporarily alleviate symptoms and vary widely in effectiveness among patients. Current interventional therapies include anti-inflammatory drugs such as corticosteroids, immunosuppressants, and passive immunotherapies (monoclonal antibodies) against inflammatory mediators such as the cytokines TNF5 or IL-12/IL-236. These therapies, however, require ongoing administration throughout this life-long disease and are critically reliant on rigorous patient compliance. Additionally, while passive immunotherapies using monoclonal antibody therapeutics are among the more promising treatments for IBD, their drawbacks include primary non-response rates as high as 30%, the development of anti-drug antibodies (ADAs) that accounts for up to a 46% secondary non-response rate, hypersensitivity reactions, and a heightened susceptibility to severe infections. Consequently, there exists a critical unmet need for a long-lasting and broadly effective IBD treatment.

### SUMMARY

In an aspect, provided herein is phosphorylcholine (PC)-conjugate peptide. The PC-conjugate peptide may include (i) a self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and (ii) at least one PC molecule conjugated to a terminus of the self-assembling peptide. In some embodiments, each self-assembling peptide forms a beta sheet and comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn). In some embodiments, the self-assembling peptide comprises the sequence QQKFQFQFEQQ (SEQ ID NO: 31) orAc-QQKFQFQFEQQ-NH₂ (SEQ ID NO: 99). In some embodiments, each self-assembling peptide forms an alpha-helix and comprises a polypeptide having an amino acid sequence of bXXXb (SEQ ID NO: 1), wherein X is independently any amino acid and b is independently any positively charged amino acid. In some embodiments, b is independently selected from Arg and Lys. In some embodiments, bXXXb (SEQ ID NO: 1) is RAYAR (SEQ ID NO: 2) or KAYAK (SEQ ID NO: 3). In some embodiments, the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 22), or Ac-QAKILEADAEILKAYAKILEAHAEILKAQ-NH₂ (SEQ ID NO: 23), or Ac-ADAEILRAYARILEAHAEILRAQ-NH₂ (SEQ ID NO: 24). In some embodiments, the conjugate further includes (iii) a linker between the at least one PC molecule and the self-assembling peptide. In some embodiments, the linker comprises an amino acid sequence selected from SEQ ID NO: 25 ((Ser-Gly)₂), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), SEQ ID NO: 29 (EAAAK)₂, and SEQ ID NO: 30 (GGAAY). In some embodiments, the linker comprises (Ser-Gly)₂ (SEQ ID NO: 25). In some embodiments, each PC molecule is attached to the linker via a Cys residue or an Asn residue. In some embodiments, the at least one PC molecule is attached to the C-terminus or the N-terminus of the self-assembling peptide. In some embodiments, 1 to 10 PC molecules are attached to the C-terminus or the N-terminus of the self-assembling peptide. In some embodiments, each PC molecule is independently attached to the linker via a Cys residue or an Asn residue. In some embodiments, 1 to 10 PC molecules are attached to the C-terminus or the N-terminus of the self-assembling peptide via a single linker. In some embodiments, 1 to 4 PC molecules are attached to the C-terminus or the N-terminus of the self-assembling peptide.

In some embodiments, 4 PC molecules are attached to the C-terminus or the N-terminus of the self-assembling peptide. In some embodiments, the conjugate comprises the sequence of (PC molecule)₄-CCCCSGSG-QQKFQFQFEQQ-NH₂ SEQ ID NO: 34), wherein each one of the four PC molecules is attached to a different Cys in the linker CCCCSGSG (SEQ ID NO: 26). In some embodiments, each PC molecule is independently selected from C₅H₁₄NO₄P and C₁₁H₂₂NO₆P. In some embodiments, the PC-conjugate peptide comprises a sequence selected from C₅H₁₄NO₄P-SGSG-QQKFQFQFEQQ-NH₂ (pa-PC-Q11, SEQ ID NO: 32), or C₁₁H₂₂NO₆P-CSGSG-QQKFQFQFEQQ-NH₂ (PC-Q11, SEQ ID NO: 33), or (C₁₁H₂₂NO₆P)ₙ-CCCCSGSG-QQKFQFQFEQQ-NH₂ (PC_{M}-Q11, SEQ ID NO: 34) wherein n is an integer selected from 1, 2, 3, and 4). In some embodiments, the conjugate further includes a PAS peptide of SEQ ID NO: 35 or 36 conjugated to the self-assembling peptide at the opposite terminus from wherein the PC molecule is attached.

In another aspect, provided herein is a nanofiber comprising a plurality of PC-conjugate as detailed herein, wherein the conjugate peptide self-assembles into the nanofiber.

In another aspect, provided is a nanofiber comprising (i) at least one PC-conjugate peptide as detailed herein; and (ii) at least one PADRE-conjugate peptide comprising: a self-assembling peptide, wherein each self-assembling peptide independently comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and at least one PADRE molecule, wherein at least one self-assembling peptide is conjugated to at least one PADRE molecule, and wherein the PADRE molecule comprises a polypeptide having the amino acid sequence of aKXVAAWTLKAa (SEQ ID NO: 18, wherein “X” comprises cyclohexylalanine and “a” comprises D-alanine). In some embodiments, the PADRE-conjugate peptide further comprises a linker between the PADRE molecule and the self-assembling peptide. In some embodiments, the linker comprises an amino acid sequence selected from SEQ ID NO: 25 ((Ser-Gly)₂), SEQ ID NO: 26 (CCCCSGSG), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), and SEQ ID NO: 29 (EAAAK)₂, and SEQ ID NO: 30 (GGAAY). In some embodiments, the at least one PADRE-conjugate peptide comprises the sequence of NH₂-aKXVAAWTLKAa-SGSG-QQKFQFQFEQQ-NH₂ (PADRE-Q11, SEQ ID NO: 37). In some embodiments, the cyclohexylalanine comprises D-alanine. In some embodiments, the nanofiber includes (iv) a plain self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid), without any PC molecules attached thereto. In some embodiments, the nanofiber comprises a combination of 10 to 10,000, or 100 to 10,000 peptides comprising PC-conjugate peptides and PADRE-conjugate peptides. In some embodiments, the nanofiber comprises a combination of 10 to 10,000, or 100 to 10,000 peptides comprising PC-conjugate peptides, PADRE-conjugate peptides, and plain self-assembling peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are PC-conjugate peptides. In some embodiments, at least about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the peptides in the nanofiber are PADRE-conjugate peptides. In some embodiments, at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides. In some embodiments, about 50% of the peptides in the nanofiber are PC-conjugate peptides, about 2.5% of the peptides are PADRE-conjugate peptides, and about 47.5% of the peptides are plain self-assembling peptides. In some embodiments, the PC-peptide conjugate and the PADRE-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1. In some embodiments, the self-assembling peptide forms a fibril including beta-sheet structures. In some embodiments, the self-assembling peptide forms a fibril having a coiled coil structure. In some embodiments, the self-assembling peptide forms a fibril having a structure of a helical filament formed around a central axis. In some embodiments, the N-terminus of each self-assembling peptide is positioned at the exterior of the helical filament. In some embodiments, the PC molecules are exposed on the exterior surface of the nanofiber. In some embodiments, the nanofiber is about 5-20 nm in width. In some embodiments, the nanofiber is about 100 nm to 1 μm, 100 nm to 2 μm, 100 nm to 3 μm, 100 nm to 4 μm, or 100 nm to 5 μm in length.

In another aspect, provided is a phosphorylcholine (PC)-conjugate peptide comprising (i) a self-assembling peptide, wherein the self-assembling peptide comprises a polypeptide having an amino acid sequence selected from VEVKVEVKV (SEQ ID NO: 65), VEVKVEVKVEVK (SEQ ID NO: 66), VVVAAAEEE (SEQ ID NO: 67), VEVEVEVEVEVEVEVEVEVE (SEQ ID NO: 68), CGNKRTRGC (SEQ ID NO: 69), VKVKVKVKVDPPTKVEVKVKV (SEQ ID NO: 70), LRKKLGKA (SEQ ID NO: 71), VVVVVVKK (SEQ ID NO: 72), AEAKAEAKAEAKAEAK (SEQ ID NO: 47), AEAKAEAK (SEQ ID NO: 73), AEAEAEAEAKAK (SEQ ID NO: 74), AEAEAKAK (SEQ ID NO: 75), AEAEAKAKAEAEAKAK (SEQ ID NO: 48), RADARADARADARADA (SEQ ID NO: 50), RADARGDARADARGDA (SEQ ID NO: 76), RADARADA (SEQ ID NO: 77), RARADADARARADADA (SEQ ID NO: 51), RARADADA (SEQ ID NO: 78), RARARARADADADADA (SEQ ID NO: 79), ADADADADARARARAR (SEQ ID NO: 80), DADADADARARARARA (SEQ ID NO: 81), RAEARAEARAEARAEA (SEQ ID NO: 82), RAEARAEA (SEQ ID NO: 83), KAKAKAKAEAEAEAEA (SEQ ID NO: 84), AEAEAEAEAKAKAKAK (SEQ ID NO: 49), KADAKADAKADAKADA (SEQ ID NO: 85), KADAKADA (SEQ ID NO: 86), AEAEAHAHAEAEAHAHA (SEQ ID NO: 87), AEAEAHAHA (SEQ ID NO: 88), HEHEHKHKHEHEHKHK (SEQ ID NO: 89), HEHEHKHK (SEQ ID NO: 90), FEFEFKFKFEFEFKFK (SEQ ID NO: 91), FEFKFEFK (SEQ ID NO: 92), LELELKLKLELELKLK (SEQ ID NO: 93), LELELKLK (SEQ ID NO: 94), KFDLKKDLKLDL (SEQ ID NO: 95), FKFEFKFF (SEQ ID NO: 96), FEFEFKFK (SEQ ID NO: 97), and RFRFRFRFRFRFRFRFRFRF (SEQ ID NO: 98); and (ii) at least one PC molecule conjugated to a terminus of the self-assembling peptide. In some embodiments, a plurality of the PC-peptide conjugates assembles into a nanofiber, nanotube, hydrogel, micelle, vesicle, nanoparticle, or suspension.

In another aspect, provided is a pharmaceutical composition comprising (a) a PC-conjugate peptide as detailed herein or a nanofiber as detailed herein; and (b) a pharmaceutically acceptable carrier, diluent, and/or excipient.

In another aspect, provided is a method of stimulating B1a cells to express antibodies. The method may include contacting at least one B1a cell with a PC-conjugate peptide as detailed herein or a nanofiber as detailed herein, such that the B1a cells in the subject express antibodies.

In another aspect, provided is a method of producing anti-PC antibodies in a subject. The method may include administering to the subject an effective amount of a PC-conjugate peptide as detailed herein or a nanofiber as detailed herein or a pharmaceutical composition as detailed herein, such that anti-PC antibodies are produced in the subject. In some embodiments, the antibodies are selected from IgM, IgG, and IgA, or a combination thereof.

In another aspect, provided is a method of treating an inflammatory disease or disorder in a subject. The method may include administering to the subject a therapeutically effective amount of a PC-conjugate peptide as detailed herein or a nanofiber as detailed herein or a pharmaceutical composition as detailed herein. In some embodiments, the inflammatory disease or disorder comprises inflammatory bowel disease (IBD), ulcerative colitis, Crohn's disease, rheumatoid arthritis, psoriasis, cirrhosis, atherosclerosis, cardiovascular inflammation, or a combination thereof.

In another aspect, provided is a method of altering gut microbiome activity in a subject. The method may include administering to the subject a therapeutically effective amount of a conjugate peptide as detailed herein or a nanofiber as detailed herein or a pharmaceutical composition as detailed herein.

In some embodiments, the bacterial diversity in the colon is reduced. In some embodiments, the amount of viable bacteria spread to the spleen from the colon is reduced. In some embodiments, the PC-peptide conjugate or the nanofiber is administered to the subject intraperitoneally, orally, sublingually, intravenously, nasally, buccally, transdermally, intranasally, or intramuscularly, or via the lung. In some embodiments, the method further includes administering at least one additional therapeutic agent. In some embodiments, the at least one additional therapeutic agent is administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition. In some embodiments, the at least one additional therapeutic agent comprises an adjuvant. In some embodiments, the adjuvant comprises CpG, or cholera toxin B subunit (CTB), or STING agonist, or cyclic dinucleotides, or alum, or MF59, or a combination thereof.

The disclosure provides for other aspects and embodiments that will be apparent in light of the following detailed description and accompanying figures.

### BRIEF DESCRIPTION OF THE DRAWINGS

FIGS. 1A-1L. Nanofiber design and influence of PC conjugation chemistry and multivalency. FIG. 1A, Schematic representation of PC-bearing nanofibers shown with a coassembled T-cell epitope. FIG. 1B, Schematic of nanofiber components. FIG. 1C, Structures of pa-PC-Q11 (phosphoramidate linkage) versus PC-Q11 (phosphodiester linkage). FIG. 1D, Negative-stained TEMs showing nanofiber morphology of pa-PC-Q11 (1 mM pa-PC-Q11 coassembled with 1 mM Q11) and PC-Q11 (1 mM PC-Q11 coassembled with 1 mM Q11), both diluted 10-fold before imaging. FIG. 1E, Zeta potential of nanofibers (formulations as in D) indicates the zwitterionic surface charge expected of PC only for PC-Q11, n=3 technical replicates. FIG. 1F, Serum total anti-PC IgG responses of mice immunized s.c and i.p. at weeks 0, 3, and 11 with either pa-PC-Q11 or PC-Q11 (both coassembled with PADRE-Q11), n=5 mice. FIG. 1G, Schematic representation of PC_{M}-Q11 nanofibers shown with a coassembled T-cell epitope. FIG. 1H and FIG. 1I, Schematic and structure of PC_{M}-Q11 nanofiber component showing 4 cysteines with attachment sites for PC. FIG. 1J, Negative-stained TEMs showing nanofiber morphology of PC_{M}-Q11 (0.2 mM PC_{M}-Q11 coassembled with 1.8 mM Q11), diluted 10-fold before imaging. FIG. 1K, ThT assay illustrating β-sheet structure, n=3 technical replicates. FIG. 1L, Serum total anti-PC IgG responses of mice immunized i.p. at weeks 0, 2, and 4 with PC-Q11 or PC_{M}-Q11 (both coassembled with PADRE-Q11), n=5 mice. AUC=area under the curve. Mean±SEM are shown for FIG. 1E, FIG. 1K, and FIG. 1L. Mean+SEM are shown for FIG. 1F. Statistical significance was determined by two-way RM ANOVA with Tukey's multiple comparison test at week 12 for FIG. 1F and two-way RM ANOVA for FIG. 1L.

FIGS. 2A-2C. MALDI-TOF mass spectrometry of PC-peptide conjugates. FIG. 2A, pa-PC-Q11 with phosphoramidate PC conjugation, expected m/z of 1936.84. FIG. 2B, PC-Q11 with Michael addition conjugation that preserves phosphodiester bond, expected m/z of 2172.27. FIG. 2C, PC_{M}-Q11 showing the conjugation of 0-4 PC molecules, expected m/z of: PC₀-Q11=2186.45, PC₁-Q11=2480.72, PC₂-Q11=2772.27, PC₃-Q11=3064.52, and PC₄-Q11=3356.79.

FIGS. 3A-3D. Nanofiber widths are consistent across PC-peptide formulations. Negative-stained TEMs used for width measurements of (FIG. 3A) pa-PC-Q11 (1 mM pa-PC-Q11 coassembled with 1 mM Q11), (FIG. 3B) PC-Q11 (1 mM PC-Q11 coassembled with 1 mM Q11), and (FIG. 3C) PC_{M}-Q11 (0.2 mM PC_{M}-Q11 coassembled with 1.8 mM Q11). FIG. 3D, Nanofiber widths measured from TEMs using ImageJ. n=30 measurements.

FIGS. 4A-4B. Reactivity of PC nanofibers with an anti-PC mAb. FIG. 4A, Binding of an anti-PC mAb to PC-BSA (positive control) and PC-Q11 but not pa-PC-Q11 or Q11. FIG. 4B, Binding of an anti-PC mAb to PC-BSA (positive control), PC-Q11, and PC_{M}-Q11, but not Q11.

FIGS. 5A-5C. Intraperitoneal immunization with PC-Q11 and CpG adjuvant increases antibody and T-cell responses. Mice were immunized either i.p. or s.c. at weeks 0, 2, and 4 with PC-Q11 coassembled with PADRE-Q11 and with or without CpG adjuvant. FIG. 5A, Serum total anti-PC IgG responses showed that i.p. immunization raised the greatest responses and that these responses were vastly improved with the addition of CpG. FIG. 5B, Serum anti-PC IgM responses showed significantly greater IgM for mice immunized i.p. with CpG. FIG. 5C, Week 5 ELISpot of splenocytes restimulated with PADRE peptide showed heightened T-cell responses after immunizations containing CpG with biasing towards IFNγ for i.p. immunized mice. SFC=spot forming cell. AUC=area under the curve. Mean+ or ±SEM are shown. Statistical significance determined by two-way RM ANOVA with Tukey's multiple comparison test for FIG. 5A and FIG. 5B, two-way ANOVA with Šidák's multiple comparisons test for FIG. 5C. n=5 mice.

FIGS. 6A-6C. Additional cysteines in nanofibers do not improve anti-PC antibody responses. Negative-stained TEMs of (FIG. 6A) PC-Q11 (1 mM PC-Q11 coassembled with 0.2 mM Cys₄-Q11, 0.05 mM PADRE-Q11, and 0.75 mM Q11) and (FIG. 6B) PC_{M}-Q11 (0.2 mM PC_{M}-Q11 coassembled with 0.2 mM Cys₄-Q11, 0.05 mM PADRE-Q11, and 1.55 mM Q11) both coassembled with 10% Cys₄-Q11 showed that the addition of excess free cysteines did not inhibit nanofiber formation. FIG. 6C, Mice were immunized i.p. at weeks 0, 2, and 4 with PC-Q11 or PC_{M}-Q11 coassembled with PADRE-Q11 and with or without Cys₄-Q11. Serum total anti-PC IgG responses indicated that the addition of Cys₄-Q11 did not significantly enhance anti-PC IgG antibody levels for PC-Q11 or PC_{M}-Q11. Mean+SEM are shown. AUC=area under the curve. ns=not significant via two-way RM ANOVA with Tukey's multiple comparison test. n=5 mice.

FIG. 7. Increasing PC_{M}-Q11 epitope content 2.5′ does not increase anti-PC antibody responses. Mice were immunized i.p. at weeks 0, 2, 4, and 9 with 10% PC_{M}-Q11 as used in other experiments (0.2 mM PCM-Q11, 1.75 mM Q11, and 0.05 mM PADRE-Q11) or 25% PC_{M}-Q11 with 2.5′ the amount of PC epitope (0.5 mM PC_{M}-Q11, 1.45 mM Q11, and 0.05 mM PADRE-Q11) with CpG adjuvant. Serum total anti-PC IgG responses showed that both 10% PC_{M}-Q11 and 25% PC_{M}-Q11 raised robust anti-PC IgG responses that were not statistically different from each other via two-way RM ANOVA. AUC=area under the curve. n=5 mice.

FIG. 8. Representative flow cytometry gating scheme for nanofiber uptake. To accompany FIGS. 9A-9M. This representative sample is from a mouse i.p. injected with TAMRA-labeled Q11. Nanofiber uptake was identified in B1a cells (CD19+CD5+), other B cells (CD19+CD5−), macrophages (F4/80+) and dendritic cells (F4/80-CD11c+) via the PE channel to detect TAMRA.

FIGS. 9A-9M. PC_{M}-Q11 is taken up more selectively by Bia cells over all other B cells. Cells were isolated from i.p. lavage fluid 4 hours after i.p. injection of TAMRA-labeled nanofibers or PBS and analyzed via flow cytometry. FIGS. 9A-9D, Nanofiber uptake in non-B1a (CD5⁻) B cells (FIG. 9A), B1a (CD5⁺) cells (FIG. 9B), macrophages (FIG. 9C), and DCs (FIG. 9D) indicated that macrophages acquired all three nanofibers, large percentages of the other three cell types all acquired PC-Q11, yet B1a cells acquired PC_{M}-Q11 to a greater extent than DCs or non-B1a B cells. FIG. 9E, Average percentage of each cell type among total TAMRA⁺ cells. FIG. 9F and FIG. 9G, PC_{M}-Q11 led to fewer non-B1a B cells in the peritoneal cavity than PC-Q11 and controls (FIG. 9F) and recruited almost three times as many B1a cells as PC-Q11 (FIG. 9G). FIG. 9H and FIG. 9I, TAMRA MFI of B cells (FIG. 9H) and B1a cells (FIG. 9I). FIG. 9J, Representative histograms showing TAMRA MFI plotted against mode-normalized cell counts for B cells and B1a cells further indicated more selective uptake of PC_{M}-Q11 by B1a cells. FIG. 9K and FIG. 9L, CD86 MFI of TAMRA⁺ B cells (FIG. 9K) and B1a cells (FIG. 9L). FIG. 9M, Representative histograms showing CD86 MFI plotted against mode-normalized cell counts for TAMRA⁺ B cells and B1a cells shows that PC_{M}-Q11 more selectively activates B1a cells. Percentages are reported as the percent of the parent population. Mean±SEM shown. Statistical significance determined by one-way ANOVA with Tukey's multiple comparison test. Results combined from two experiments. n=10 mice except for Q11 where n=9 mice and FIG. 9K and FIG. 9L where n=6 mice.

FIGS. 10A-10M. Addition of the T-cell epitope, PADRE, and CpG adjuvant to PC immunizations broadens the antibody subclasses produced and enhances T-cell responses. FIGS. 10A-10C, Mice were immunized i.p. at weeks 0, 2, and 4 with PC-Q11 or PC_{M}-Q11 with or without coassembled PADRE-Q11. FIG. 10A, Serum total anti-PC IgG responses. FIG. 10B, Serum anti-PC IgM and IgG subclass responses at week 5. FIG. 10C, ELISpot of splenocytes stimulated with PADRE peptide indicated no significant T-cell response after i.p. immunization. FIGS. 10D-10F, Mice were immunized i.p. at weeks 0, 2, and 4 with CpG-adjuvanted PC-Q11 or PC_{M}-Q11 with or without coassembled PADRE-Q11. FIG. 10D, Serum total anti-PC IgG responses. FIG. 10E, Serum anti-PC IgM and IgG subclass responses at week 5. FIG. 10F, ELISpot of splenocytes stimulated with PADRE peptide showed significant T-cell response after i.p. immunization for PADRE-containing immunizations. FIGS. 10G-10M, Mice were injected i.p. with either an anti-CD4 depletion or isotype control antibody at days −1, −3, 3, 7, 11, 15, and 19 and immunized i.p. at weeks 0 (day 0) and 2 (day 14) with CpG-adjuvanted PC-Q11 or PC_{M}-Q11 coassembled with PADRE-Q11. FIGS. 10G-10H, Serum anti-PC IgM was produced regardless of CD4⁺ T-cell presence. FIGS. 101-10J, Serum total anti-PC IgG production was significantly dependent on CD4⁺ T cells. FIGS. 10K-10L, Serum anti-PC IgG subclass responses at week 3 show that IgG3 is the main subclass produced in CD4-depleted mice. FIG. 10M, PADRE-specific T-cell responses are present in isotype control antibody administered mice. AUC=area under the curve. SFC=spot forming cell. Mean+ or ±SEM are shown. Statistical significance was determined by two-way RM ANOVA with Tukey's multiple comparison test for FIG. 10A and FIG. 10D, two-way ANOVA with Tukey's multiple comparison test for FIG. 10B, FIG. 10E, FIG. 10K, and FIG. 10L, two-way ANOVA with Šidák's multiple comparison test for FIG. 10C, FIG. 10F, and FIG. 10M, and two-way RM ANOVA for FIGS. 10G-10J. n=10 mice for FIG. 10A, FIG. 10B, FIG. 10D, and FIG. 10E, and n=5 mice for FIG. 10C, FIG. 10F, and FIG. 10G-10M.

FIGS. 11A-11DE. Administration of an anti-CD4 monoclonal antibody depletes the CD4⁺ T cell population. FIG. 11A, Timeline of CD4⁺ T-cell depletion experiment. Mice were injected i.p. with 200 μg of either anti-CD4 or isotype control antibody on days −3, 1, 3, 7, 11, 15, and 19 and immunized i.p. on day 0 and 14 with PC-Q11 or PC_{M}-Q11 coassembled PADRE-Q11 and CpG adjuvant. FIG. 11B, FIG. 11C, Representative flow plots verifying CD4⁺ T cell depletion in unimmunized mice at (FIG. 11B) day 0 and (FIG. 11C) day 22 in the spleen, draining lymph nodes (axial, brachial, and inguinal), and mesenteric lymph nodes. FIG. 11D, FIG. 11E, Representative histograms of the CD4+ population in naïve or depleted mice at (FIG. 11D) day 0 and (FIG. 11E) day 22. n=2 mice per group per timepoint.

FIG. 12. Representative flow cytometry gating scheme for CD4+ T cell depletion. FIG. 12 accompanies FIGS. 10A-10M and FIGS. 11A-11E. This representative scheme is from an isotype control antibody-administered mouse.

FIGS. 13A-13H. Immunization with PC_{M}-Q11 is protective in a model of chronic colitis. FIG. 13A, Timeline of chronic colitis experiment. Mice were immunized i.p. at days −35, −21, and −7 with either PC_{M}-Q11 (coassembled with PADRE-Q11) with or without CpG adjuvant. Control mice received PBS immunizations. Chronic colitis was induced on day 1 via administration of 2% DSS (w/v) for 5 days followed by 5 days of normal drinking water for 3 cycles at which point mice were euthanized. FIG. 13B, Serum total anti-PC IgG responses. FIG. 13C, Serum anti-PC IgM. FIG. 13D, Serum anti-PC IgG subclass responses at week 5, before beginning DSS administration. FIG. 13E, Daily mouse body weights as a percentage of their day 0 weight. FIG. 13F, Daily disease activity index (DAI) scores as a combination of weight loss, fecal score, and occult blood, with high scores indicating more severe disease. FIG. 13G, Colon lengths were significantly longer for immunized groups compared to DSS disease controls. FIG. 13H, IL-10 levels of colon homogenates measured via ELISA. Results combined from two experiments except for IL-10 data (FIG. 13H), which is from chronic colitis experiment 1. Mean+, − or ±SEM are shown. Statistical significance determined by two-way RM ANOVA for FIG. 13B and FIG. 13C, two-way ANOVA with Tukey's multiple comparison test for FIG. 13D, mixed-effects analysis with Tukey's multiple comparison test for FIG. 13E and FIG. 13F, and one-way ANOVA with Tukey's multiple comparison test for FIG. 13G and FIG. 13H. n=20 mice except for FIG. 13H where n=10 mice, ns=not significant.

FIGS. 14A-14B. Serum total anti-PC IgG antibodies for control groups in chronic DSS model. Mice were injected i.p. at weeks 0, 2, and 4 with PBS and chronic colitis was induced at week 5 via administration of 2% DSS for 5 days followed by 5 days of normal drinking water for 3 cycles at which point mice were euthanized. FIG. 14A, Serum total anti-PC IgG measurements showed little to no detectable antibodies in PBS with DSS (disease) or PBS without DSS (healthy) control mice. FIG. 14B, Little to no anti-PC IgM responses were above detection levels. n=20 mice.

FIGS. 15A-15F. Protective efficacy of immunization with PC_{M}-Q11 is reproducible in two models of chronic colitis. Shown are separated colitis data from two chronic experiments that was combined in FIGS. 13A-131. FIGS. 15A-15C, Chronic colitis experiment 1. FIGS. 15D-15F, Chronic colitis experiment 2. For both experiments, mice were immunized i.p. at days −35, −21, and −7 with either PC_{M}-Q11 (coassembled with PADRE-Q11) with or without CpG adjuvant. Control mice received PBS immunizations. Chronic colitis was induced on day 1 via administration of 2% DSS (w/v) for 5 days followed by 5 days of normal drinking water for 3 cycles at which point mice were euthanized. FIG. 15A, FIG. 150, Daily mouse body weights as a percentage of their day 0 weight. FIG. 15B, FIG. 15E, Daily disease activity index (DAI) scores as a combination of weight loss, fecal score, and occult blood, with high scores indicating more severe disease. FIG. 15C, FIG. 15F, Colon lengths were significantly longer for immunized groups compared to DSS disease controls. Mean+, − or ±SEM are shown. Statistical significance determined by two-way RM ANOVA for FIG. 15A, mixed-effects analysis with Tukey's multiple comparison test for FIG. 15B, FIG. 15D and FIG. 15E, and one-way ANOVA with Tukey's multiple comparison test for FIG. 15C and FIG. 15F. n=10 mice.

FIG. 16. Chronic DSS colitis significantly shortens colons. Colon images from chronic DSS experiment 2 indicates shortened colons for mice exposed to DSS, with this being the most apparent in the PBS with DSS group. An image of mouse 2 from the PBS with DSS group is missing (mouse reached humane endpoints on day 28, 2 days before the planned experimental endpoint). n=10 mice except the PBS with DSS group where n=9 mice.

FIGS. 17A-17B. Bacteria cultured from the spleen and serum FITC-dextran levels were not significantly altered after chronic colitis compared to healthy controls. FIG. 17A, Spleen CFUs, an indication of bacterial spread from colon damage, were not different among groups. FIG. 17B, Measures of FITC-dextran in serum after oral gavage were not different among groups. Data from chronic colitis experiment 2. Mean+, − or ±SEM are shown. Lack of statistical significance determined by one-way ANOVA with Tukey's multiple comparison test. n=10 mice.

FIGS. 18A-18L. Immunization with PC_{M}-Q11 does not significantly improve histological damage 5 days after the last round of DSS administration in chronic colitis. FIGS. 18A-18H, Representative images are shown of the proximal colon (left) and the distal colon (right) for the following groups: PC_{M}-Q11 (FIG. 18A, FIG. 18B), PC_{M}-Q11/CpG (FIG. 18C, FIG. 18D), PBS with DSS (FIG. 18E, FIG. 18F), and PBS without DSS (FIG. 18G, FIG. 18H). At this time point, all mice exposed to DSS show expansion of the mucosa by acute inflammation, extending at least into the submucosa, as well as architectural distortion characteristic of chronic colitis (FIG. 18A, FIG. 18C, FIG. 18E), and at least focal ulcerations (* in FIG. 18B, FIG. 18D, FIG. 18F). Scale bar=200 μm. FIG. 18I, FIG. 18J, Segment scores for proximal (FIG. 18I) and distal (FIG. 18J) colons. FIG. 18K, FIG. 18L, Extent of severe changes score for the proximal (FIG. 18K) and distal (FIG. 18L) colon segments show a slight though not significant trend of decreased scores for the PC_{M}-Q11/CpG immunized group. Data from chronic colitis experiment 2. Mean±SEM are shown. Statistical significance determined by one-way ANOVA with Tukey's multiple comparison test. n=10 mice.

FIGS. 19A-19G. Protective effects of PC_{M}-Q11 immunization repeat through one cycle of DSS colitis in males and females, reduce bacterial spread after colon damage, and are not attributed to CpG alone. FIG. 19A, Timeline of one cycle of DSS colitis experiment. Mice were immunized i.p. at days −35, −21, and −7 with either PC_{M}-Q11 (coassembled with PADRE-Q11) with or without CpG, CpG alone, or PBS (control mice). Colitis was induced on day 1 via administration of 2% DSS (w/v) for 5 days followed by 5 days of normal drinking water, concluding at day 10. FIG. 19B, Serum total anti-PC IgG responses. FIG. 19C, Daily mouse weights as a percentage of their original weight, with statistical comparisons shown below. FIG. 19D, DAI scores as a combination of weight loss, fecal score, and occult blood, with statistical comparisons shown below. FIG. 19E, Colon lengths were significantly longer for immunized groups compared to CpG alone and DSS disease controls. FIG. 19F, Spleen CFUs indicate that immunizations prevented colon damage-associated bacterial spread to the spleen, although this effect was more pronounced in female mice. FIG. 19G, IL-10 levels of colon homogenates showed no significant difference among disease groups; however, PC_{M}-Q11 immunized IL-10 colon levels in males were also not significantly different from healthy controls. Mean+, − or ±SEM are shown. PBS without DSS control groups in FIGS. 19C-19G female mice and FIGS. 30C-30G are the same data, as these experiments were run in parallel. Female and male experiments were conducted separately. Statistical significance was determined by two-way RM ANOVA with Tukey's multiple comparison test for FIG. 19B and FIG. 19C female, mixed-effects analysis with Tukey's multiple comparison test for FIG. 19C male and FIG. 19D, and one-way ANOVA with Tukey's (FIG. 19E and FIG. 19G) or Dunnett's (to PBS with DSS disease control) (FIG. 19F) multiple comparison test for FIGS. 19E-19G. n=5 or 4 mice as 1 mouse each in the male PBS with DSS and PC_{M}-Q11 groups reached humane endpoint before the experimental endpoint, ns=not significant.

FIGS. 20A-20C. Serum anti-PC antibody responses for the one cycle DSS model. FIG. 20A, Serum anti-PC IgM responses for PC_{M}-Q11 and CpG were undetectable in female mice unlike PC_{M}-Q11/CpG immunized mice. Male mice immunized with PC_{M}-Q11 had significantly less anti-PC IgM than PC_{M}-Q11/CpG immunize mice. FIG. 20B, FIG. 20C, Serum antibody responses for PBS with DSS (disease) or PBS without DSS (healthy) control mice showed only one mouse with detectable anti-PC IgG (FIG. 20B) and no mice with detectable anti-PC IgM (FIG. 20C) for both females and males. Mean±SEM are shown. Statistical significance determined by two-way RM ANOVA. n=5 mice.

FIGS. 21A-211. PC_{M}-Q11 immunizations reduce disease severity when administered therapeutically between DSS-colitis cycles. FIG. 21A, Timeline of therapeutic DSS colitis experiment. Colitis was induced by administering one cycle of DSS colitis achieved by 5 days of 2% DSS in water followed by normal drinking water. Mice were monitored daily until they on average regained their original body weight on day 20. Mice were immunized i.p. with PC_{M}-Q11 co-assembled with PADRE-Q11 and with or without CpG on days 20, 32, and 45; colitis symptoms were monitored twice weekly. An additional cycle of colitis was then induced on day 51 via 5 days of 2% DSS in water followed by 5 days of normal drinking water for a total of 10 days with daily monitoring until the experimental endpoint on day 60. FIGS. 21B-21D, Serum total anti-PC IgG (FIG. 21B), IgM (FIG. 21C), and IgG subclass (FIG. 21D) responses. FIG. 21E, Daily mouse weights as a percentage of their original weight. FIG. 21F, DAI scores as a combination of weight loss, fecal score, and occult blood (* on day 26 indicates significantly higher DAI scores in the PBS with DSS group, with P=0.0039 to PC_{M}-Q11, P=0.0157 to PC_{M}-Q11/CpG, and P=0.0003 to PBS without DSS). FIG. 21G, Colon lengths were significantly longer for immunized groups compared to DSS disease controls. FIG. 21H, Spleen CFUs indicate that immunizations prevented colon damage-associated bacterial spread and were similar to healthy controls. FIG. 21I, FITC-dextran serum measurements taken 3 hours after oral gavage of 3-5 kDa m.w. FITC-dextran on day 60. Mean+, − or ±SEM are shown. Statistical significance was determined by two-way RM ANOVA for FIG. 21B and FIG. 21C, two-way ANOVA with Tukey's multiple comparison test for FIG. 21D, two-way RM ANOVA with Tukey's multiple comparison test at day 60 for FIG. 21E, two-way RM ANOVA from day 50-60 with Tukey's multiple comparison test for FIG. 21F, and one-way ANOVA with Tukey's (FIG. 21G, FIG. 21I) or Dunnett's (to PBS with DSS disease control) (FIG. 21H) multiple comparison test for FIGS. 21G-21I. n=10 mice.

FIGS. 22A-22B. Serum anti-PC antibody responses for therapeutic DSS model. Serum anti-PC IgG (FIG. 22A) and IgM (FIG. 22B) antibody responses for PBS with DSS (disease) or PBS without DSS (healthy) control mice during the therapeutic colitis model. n=10 mice.

FIG. 23. Therapeutic DSS colitis colon images. Colon images from the therapeutic DSS experiment showed shortened colons for mice exposed to DSS, with this being most apparent in the PBS with DSS group. n=10 mice.

FIGS. 24A-24J. PC_{M}-Q11 immunizations reduce the incidence of ulceration in the proximal colon but overall do not significantly improve histology scores in the therapeutic colitis DSS model. FIGS. 24A-24D, Representative images are shown of the proximal colon from the following groups: PC_{M}-Q11 (FIG. 24A), PC_{M}-Q11/CpG (FIG. 24B), PBS with DSS (FIG. 24C), and PBS without DSS (FIG. 24D). At this time point, all mice exposed to DSS showed acute inflammation extending into the submucosa, although ulcerations in the proximal colon are seen only in the non-immunized mice exposed to DSS (* in FIG. 24C). Scale bar=200 μm. FIG. 24E, FIG. 24F, Ulceration length per segment length in the proximal (FIG. 24E) and distal (FIG. 24F) colon segments showed fewer ulcerations present in the proximal colon for immunized groups. Segment scores for proximal (FIG. 24G) and distal (FIG. 24H) colons. FIG. 24I, Inflammation scores from the proximal colon segments. FIG. 24J, Extent of severe changes score for the proximal colon segments show a slight though not significant trend of decreased scores for the PC_{M}-Q11/CpG immunized group. Mean±SEM are shown. Statistical significance determined by one-way ANOVA with Tukey's multiple comparison test. n=10 mice.

FIGS. 25A-25K. PC immunization reduces bleeding but does not prevent significant histological damage in an Il10^{-/-} colitis model. FIG. 25A, Timeline of Il10^{-/-} colitis model where mice were immunized i.p. on days −35, −21, and −7 with PC_{M}-Q11 coassembled with PADRE-Q11 and CpG adjuvant or PBS (disease control) followed by piroxicam administration in food fines for 7 days to trigger colitis and then 16 additional days of symptom monitoring. Robust anti-PC IgG (FIG. 25B) and IgM (FIG. 25C) antibody responses were generated in Il10^{-/-} mice. FIG. 25D, IgG subclasses elicited showed the same biasing as in wild-type C57BL/6 mice immunized with PC_{M}-Q11/CpG. There was no significant difference in weight loss (FIG. 25E) or fecal consistency (FIG. 25F) scores between piroxicam-administered groups. FIG. 25G, PC_{M}-Q11/CpG immunization significantly improved bleeding scores compared to disease controls. FIG. 25H, Both PC_{M}-Q11/CpG and PBS-administered mice given piroxicam developed moderate colitis levels as indicated by total histology scores. Hematoxylin and eosin-stained sections showed similar colon mucosal hyperplasia and inflammation in Il10^{-/-} mice exposed to piroxicam, whether immunized with PC_{M}-Q11/CpG (FIG. 25I) or with PBS alone (FIG. 25J). Mice immunized with PC_{M}-Q11/CpG but not exposed to piroxicam had minimal to no colitis (FIG. 25K). Distal colon is shown for all mice. Scale bar indicates 200 μm. Mean+, − or ±SEM are shown. Statistical significance was determined by mixed-effects analysis with Tukey's multiple comparison test for FIG. 25B and FIG. 25C and FIGS. 25E-25G, two-way ANOVA with Tukey's multiple comparison test for FIG. 25D, and one-way ANOVA with Tukey's multiple comparison test for FIG. 25H. n=10 mice for PC_{M}-Q11/CpG with piroxicam group or n=9 mice for other groups.

FIGS. 26A-26P. Levels of B cells, B1a cells, and plasma cells in the colons of mice after immunization, after DSS-induced colitis, and after piroxicam-induced colitis. FIGS. 26A-26D, CD19⁺ B cells were isolated from the colon after: (FIG. 26A) immunization, (FIG. 26B) chronic DSS colitis, (FIG. 26C) therapeutic DSS colitis, and (FIG. 26D) Il10^{-/-} piroxicam-induced colitis. FIGS. 26E-26H, B220⁺ B cells were isolated from the colon after: (FIG. 26E) immunization, (FIG. 26F) chronic DSS colitis, (FIG. 26G) therapeutic DSS colitis, and (FIG. 26H) Il10^{-/-} piroxicam-induced colitis. FIGS. 26I-26L, CD19⁺CD5⁺ B1a cells were isolated from the colon after: (FIG. 26I) immunization, (FIG. 26J) chronic DSS colitis, (FIG. 26K) therapeutic DSS colitis, and (FIG. 26L) Il10^{-/-} piroxicam-induced colitis. FIGS. 26M-26P, B220⁺CD138⁺ plasma cells were isolated from the colon after: (FIG. 26M) immunization, (FIG. 26N) chronic DSS colitis, (FIG. 26O) therapeutic DSS colitis, and (FIG. 26P) Il10^{-/-} piroxicam-induced colitis. Percentages are reported as the percent of the parent population. Mean±SEM shown. Statistical significance was determined by one-way ANOVA with Tukey's multiple comparison test. For immunization group, n=5 mice, for chronic DSS colitis, n=10 mice except for the PBS with DSS group where n=9 mice, for therapeutic colitis, n=10 mice, and for Il10^{-/-} piroxicam-induced colitis, n=9 mice except for the PC_{M}-Q11/CpG with piroxicam group where n=10 mice.

FIG. 27. Representative flow cytometry gating scheme for colon B cell, B1a cell, and plasma cell identification. FIG. 27 accompanies FIGS. 26A-26P. This representative sample was from a mouse in the therapeutic colitis model with gating after live cells to CD19⁺ (B cells) and then one step further to CD19⁺CD5⁺ (B1a cells) or to B220⁺ (B cell lineage) and then one step further to B220⁺CD138⁺ (plasma cells).

FIGS. 28A-28F. Levels of the tight junction proteins ZO-1 and occludin in colon sections after immunization and after chronic DSS colitis. FIGS. 28A-28C, Immunization with PC_{M}-Q11 coassembled with PADRE-Q11 and with or without CpG adjuvant at weeks 0, 2, and 4, does not significantly alter ZO-1 or occludin levels in the colon at week 5. FIG. 28A, Representative images showing colon section overviews (top row, scale bar=500 μm) and region of interest ZO-1 and occludin staining (bottom two rows, scale bar=50 μm). FIG. 28B, FIG. 28C, ZO-1 (FIG. 28B) and occludin (FIG. 28C) levels are not significantly different in immunized mice versus healthy controls. FIGS. 28D-28F, ZO-1 and occludin levels in the colon after chronic colitis experiment 2. FIG. 28D, Representative images showing colon section overviews (top row, scale bar=500 μm) and region of interest ZO-1 and occludin staining (bottom two rows, scale bar=50 μm). FIGS. 28E-28F, ZO-1 (FIG. 28E) and occludin (FIG. 28F) levels varied slightly among groups with PC_{M}-Q11/CpG administered mice have significantly less ZO-1 and occludin than some other groups. Mean±SEM shown. Statistical significance determined by one-way ANOVA with Tukey's multiple comparison test. n=5 mice. DAPI=blue, ZO-1=green, and occludin=red.

FIGS. 29A-29H. Gut microbiome diversity is decreased by both PC_{M}-Q11 immunization and DSS administration. Fecal samples from 5 mice in each group from the chronic DSS model experiment 1 were collected either at week 0 (naïve), week 5 (pre-DSS), or week 9 (post-DSS) and analyzed by sequencing the V4 variable region of the 16S rRNA gene. FIG. 29A, FIG. 29B, Family-level taxonomic histogram illustrating relative abundance of gut microbiome (FIG. 29A) before and after PC_{M}-Q11 (coassembled with PADRE-Q11) immunization with and without CpG and (FIG. 29B) after DSS administration. FIG. 29C, Phylum and family names of bacteria in taxonomic histogram in order of decreasing abundance. FIG. 29D, Jaccard Emperor plot showing clustering of groups based on beta diversity. FIGS. 29E-29F, Measures of alpha diversity including (FIG. 29E) observed OTUs and (FIG. 29F) Shannon diversity index for immunized groups before and after immunization and after DSS. FIG. 29G, FIG. 29H, Measures of alpha diversity including (FIG. 29G) observed OTUs and (FIG. 29H) Shannon diversity index comparing healthy water control mice and immunized or disease control mice after DSS administration. Mean±SEM are shown. Statistical significance was determined by two-way ANOVA with Tukey's multiple comparison test for FIG. 29E and FIG. 29F, and one-way ANOVA with Tukey's multiple comparison test for FIG. 29G and FIG. 29H. n=5 mice.

FIGS. 30A-30J. PC-immunized serum significantly reduces DAI scores, increases colon length when passively transferred, and binds late apoptotic cells. FIG. 30A, Timeline of serum passive transfer (PT) with one cycle of DSS colitis experiment. Sera from mice immunized i.p. with either PC_{M}-Q11 (coassembled with PADRE-Q11) with or without CpG adjuvant or PBS (naïve) was transferred i.v. at day 0 and colitis was induced on day 1 via administration of 2% DSS (w/v) for 5 days followed by 5 days of normal drinking water and ending on day 10. FIG. 30B, Serum total anti-PC IgG responses collected immediately before DSS administration and at the experimental endpoint. FIG. 30C, Daily weight loss showed no difference among disease groups. FIG. 30D, Daily DAI scores indicated reduced scores for immunized serum PT groups compared to naïve serum PT. FIG. 30E, Colon lengths were longer for immunized serum PT groups compared to the naïve serum PT control. FIG. 30F, Spleen CFUs were not different among groups. FIG. 30G, Colon IL-10 levels showed no significant difference among disease groups. FIGS. 30H-30J Caco2 colon epithelial cells were given DSS for 24 hours to induce apoptosis and then treated with PBS or pooled serum from mice immunized with either PC_{M}-Q11 (coassembled with PADRE-Q11) with or without CpG adjuvant or PBS (naïve serum). Anti-mouse IgM or IgG binding to apoptotic cells was then determined via flow cytometry. FIG. 30H, There was significantly more anti-mouse IgM detected on apoptotic cells treated with serum from PC_{M}-Q11/CpG immunized mice than naïve serum or PBS controls. FIG. 30I, There was significantly more anti-mouse IgG detected on apoptotic cells treated with serum from PC_{M}-Q11 and PC_{M}-Q11/CpG immunized mice compared to naïve serum or PBS controls. FIG. 30J, Representative histograms showing either anti-mouse IgM or IgG MFI in live versus late apoptotic cells. Mean+, − or ±SEM are shown. PBS without DSS control groups in FIGS. 19C-19G female mice and FIGS. 30C-30G are the same data, as these experiments were run in parallel. Statistical significance was determined by two-way RM ANOVA with Tukey's multiple comparison test for FIG. 30C, mixed-effects analysis with Tukey's multiple comparison test for FIG. 30D, and one-way ANOVA with Tukey's (FIG. 30E and FIGS. 30G-30I) or Dunnett's (to PBS with DSS disease control) (FIG. 30F) multiple comparison test for FIGS. 30E-30I. ns=not significant. n=5 mice for FIGS. 30A-30G or cell samples for FIG. 30H, FIG. 30I.

FIG. 31. Serum anti-PC IgM antibody responses for passive serum transfer with one cycle DSS model. Serum anti-PC IgM responses were measured in PC_{M}-Q11/CpG serum PT mice but decreased after DSS administration. No anti-PC IgM was detected in PC_{M}-Q11 serum PT or naïve (PBS injected) mice. n=5 mice.

FIG. 32. Representative flow cytometry gating scheme for apoptotic cell binding assay. FIG. 32 accompanies FIGS. 30A-30J. Late apoptotic cells were identified as DAPI⁺Apotracker⁺ and then gated further for identification of anti-mouse IgG or anti-mouse IgM positive cells.

FIG. 33 is a graph of anti-PC IgG antibodies in mice after administration with 2 mM nanofiber formulations consisting of either 0.2 mM (10%) or 1 mM (50%) PC_{M}-Q11-PAS coassembled with PAS-Q11 (1.75 or 0.95 mM respectively) and the T-cell epitope PADRE-Q11 (0.05 mM). It was found that mice given 50% PC_{M}-Q11-PAS raised anti-PC IgG responses comparable to intraperitoneal (i.p.) immunization without adjuvant.

FIG. 34 is a graph of disease activity index for mice with DSS-induced colitis and immunized either i.p. with 50% PC_{M}-Q11 and CpG adjuvant (on days 20 and 32) or orally with 50% PC_{M}-Q11-PAS with CTB adjuvant (on days 20 & 21 and 32 & 33). It was found that mice from immunized groups had significantly lower disease activity index scores on day 26 after just the initial immunizations and on day 33.

### DETAILED DESCRIPTION

Provided herein are novel peptide conjugates that self-assemble into peptide nanofibers or fibrils, with phosphorylcholine (PC) molecules conjugated thereto. The present disclosure is based, in part, on the discovery by the inventors of a novel active immunotherapy that capitalizes on native B1a cells to produce anti-PC antibodies while expanding upon the repertoire of antibodies produced. Immunization with PC as a B-cell epitope displayed on self-assembling peptide nanofibers was shown to target B1a cells and generate an anti-PC antibody response leading to ameliorated disease severity in a murine model of colitis. Immunization with optimized PC nanofibers induced both protective and therapeutic responses in the mouse models of colitis. The PC-peptide nanofibers also induced multiple classes of anti-PC antibodies through traditional immune mechanisms such as other B cell and T cell dependent mechanisms. The compositions described herein may be used, for example, to stimulate B1a cells to produce antibodies, to treat inflammatory diseases such as colitis, and alter the gut microbiome.

Active immunotherapies differ from passive immunotherapies in that they induce the production of therapeutic antibody or cellular responses by a patient instead of delivering the therapeutic antibodies directly. They are potentially advantageous for generating long-lasting therapeutic effects in the context of IBD, for example, because they may require fewer injections, reduce the possibility of anti-drug antibodies, and produce a polyclonal response with the potential for broader efficacy. The life-long endogenous production of anti-cytokine antibodies may be a potential concern if it increases vulnerability to infections. Thus, other active immunotherapy targets that can potentially circumvent this risk may be attractive. Detailed herein is a therapy capable of eliciting an antibody response emulating natural antibodies (NAbs), which play multiple roles including clearance of apoptotic debris, regulation of inflammation, and defense against pathogens. NAbs are mainly produced by B1a cells, typically in the absence of exogenous antigen. They are polyreactive and primarily of the IgM isotype, though class-switched IgG and IgA are also present. Self-renewing populations of B1a cells are largely found in the peritoneal and pleural cavities, and when they are additionally stimulated with antigen, they rapidly migrate to the spleen, differentiate into plasma cells, and begin temporarily secreting higher levels of Nab. One NAb epitope is phosphorylcholine (PC), a phospholipid head group that is externalized on cell membranes from apoptotic and injured host cells, as well as a component of some gram-negative and gram-positive bacterial cell walls and oxidized lipids. Higher levels of anti-PC NAbs may be associated with better outcomes in several autoimmune and inflammatory diseases, including systemic lupus erythematosus and atherosclerosis. Anti-PC NAbs have not been previously well-characterized in patients with IBD. As detailed herein, immunization with optimized PC nanofibers induced both protective and therapeutic responses in mouse models of colitis. A considerable proportion of this therapeutic efficacy was attributed to the induced anti-PC antibody response, thus highlighting the use in treating IBD.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

The term “adjuvant” refers to a compound or mixture that enhances the immune response to an antigen. Adjuvants may contain a substance to protect the antigen from rapid catabolism, such as aluminum hydroxide or a mineral oil, and also a protein derived from lipid A, Bortadella pertussis, or Mycobacterium tuberculosis. Suitable adjuvants may be commercially available and include, for example, complete or incomplete Freund's adjuvant; AS-2; aluminum salts such as aluminum hydroxide (as a gel, where appropriate) or aluminum phosphate; calcium salts, iron salts, or zinc salts; an insoluble suspension of acylated tyrosine; acylated sugars; cationically or anionically derivatized polysaccharides; polyphosphazenes; biologically degradable microspheres; monophosphoryl lipid A, cytokines such as GM-CSF, Interleukin-2, Interleukin-7, and Interleukin-12.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “antigen” is a molecule capable of being bound by an antibody or T-cell receptor. The term “antigen”, as used herein, also encompasses T-cell epitopes. An antigen also refers to a molecule against which a subject can initiate a humoral and/or cellular immune response leading to the activation of B-lymphocytes and/or T-lymphocytes. An antigen is capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In some embodiments, the antigen contains or is linked to a Th cell epitope. An antigen can have one or more epitopes (B-epitopes and T-epitopes). Antigens may also be mixtures of several individual antigens. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. Antigens can be microbial antigens, such as viral, fungal, or bacterial; or therapeutic antigens such as antigens associated with cancerous cells or growths, or autoimmune disorders. In some embodiments, the antigen is selected from a small molecule, nucleotide, polynucleotide, peptide, polypeptide, protein, lipid, carbohydrate, other immunogenic molecules, and a combination thereof.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Identical” or “identity” as a percentage as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Immunogenicity” refers to the ability of an antigen to induce an immune response and includes the intrinsic ability of an antigen to generate antibodies in a subject. In some embodiments, the self-assembling peptides described herein, or the nanofibers they form, are not immunogenic without an antigen such as a PC molecule appended thereto.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. Secondary structure may include beta-sheet and alpha-helices. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution. In some embodiments, a carrier includes phosphate buffered saline solution.

“Sample” or “test sample” as used herein can mean any sample in which the presence and/or level of a target is to be detected or determined or a portion from a subject or portion of an immunogenic composition as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Phosphorylcholine (PC)-Conjugate Peptide

Provided herein is a phosphorylcholine (PC)-conjugate peptide. The PC-conjugate peptide may include a self-assembling peptide and at least one PC molecule attached thereto.

a. Self-Assembling Peptide

Certain aspects include self-assembling peptides. As used herein, the term “self-assembling peptide” refers to peptides that are able to spontaneously associate and form stable structures. Each self-assembling peptide may comprise or form an alpha helix. In other embodiments, each self-assembling peptide may comprise or form a beta-sheet. Examples of self-assembling peptides are detailed in, for example, U.S. Pat. Nos. 9,241,987; 9,849,174; 10,596,238; 11,246,924, Lee, S. et al. Int. J. Mol. Sci. 2019, 20, 5850; Hernandez, A. et al. Front. Bioeng. Biotechnol. 2023, 11, 1139782; and Lopez-Silva et al. ACS Biomater. Sci. Eng. 2019, 5, 977-985, each of which is incorporated herein by reference in its entirety.

In some embodiments, the self-assembling peptide comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO: 54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn). Some examples of self-assembling peptide are detailed in, for example, U.S. Pat. Nos. 9,241,987; 9,849,174; and 10,596,238, each of which is incorporated herein by reference in its entirety. In some embodiments, the self-assembling peptide comprises the amino acid sequence of QQKFQFQFEQQ (Q11, SEQ ID NO: 31), or a polypeptide with at least 75%, 80%, 85%, or 90% identity thereto. In some embodiments, the self-assembling peptide comprises the sequence Ac-QQKFQFQFEQQ-NH₂ (Q11, SEQ ID NO: 99), or a polypeptide with at least 75%, 80%, 85%, or 90% identity thereto.

The self-assembling peptide may comprise an amino acid sequence of bXXXb (SEQ ID NO: 1), wherein X is independently any amino acid, and b is independently any positively charged amino acid. In such embodiments, each self-assembling peptide may form an alpha helix. In some embodiments, b is independently selected from Arg and Lys. In some embodiments, b is Arg. In some embodiments, b is Lys. In some embodiments, bXXXb (SEQ ID NO: 1) is RAYAR (SEQ ID NO: 2). In some embodiments, bXXXb (SEQ ID NO: 1) is KAYAK (SEQ ID NO: 3). In some embodiments, the self-assembling peptide comprises the sequence of RXXXR (SEQ ID NO: 4), wherein X is any amino acid. The self-assembling peptide may comprise an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, n is an integer from 5 to 15, and m is an integer from 5 to 15. Some examples of self-assembling peptide are detailed in, for example, U.S. Pat. No. 11,246,924, which is incorporated herein by reference in its entirety. In such embodiments, a plurality of the PC-peptide conjugates may assemble into a nanofiber.

In some embodiments, the self-assembling peptide comprises a glutamine at the C-terminus. In some embodiments, the self-assembling peptide comprises a glutamine at the N-terminus. The self-assembling peptide may include at least, at most, or exactly 5, 10, 15, 16, 17, 18, 19, 20, 21, 22, 23, 24, 25, 26, 27, 28, 29, 30, 31, 32, 33, 34, 35, or 40 amino acids. In some embodiments, the self-assembling peptide comprises 5 to 40 amino acids in length.

In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or Ac-QARILEADAEILRAYARILEAHAEILRAQ-NH₂ (SEQ ID NO: 23) or Ac-QAKILEADAEILKAYAKILEAHAEILKAQ-NH₂ (SEQ ID NO: 24) or Ac-ADAEILRAYARILEAHAEILRAQ-NH₂ (SEQ ID NO: 25, or a polypeptide with at least 75%, 80%, 85%, 90%, or 95% identity thereto. In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or a variant thereof. In some embodiments, the self-assembling peptide comprises an amino acid sequence of QARILEADAEILRAYARILEAHAEILRAQ (SEQ ID NO: 6). In some embodiments, the self-assembling peptide comprises an amino acid sequence of QAKILEADAEILKAYAKILEAHAEILKAQ (SEQ ID NO: 7). In some embodiments, the self-assembling peptide comprises an amino acid sequence of ADAEILRAYARILEAHAEILRAQ (SEQ ID NO: 8).

In some embodiments, each self-assembling peptide forms a beta sheet and comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn). In such embodiments, a plurality of the PC-peptide conjugates may assemble into a nanofiber.

In some embodiments, the self-assembling peptide comprises a polypeptide having an amino acid sequence selected from VEVKVEVKV (SEQ ID NO: 65), VEVKVEVKVEVK (SEQ ID NO: 66), VVVAAAEEE (SEQ ID NO: 67), VEVEVEVEVEVEVEVEVEVE (SEQ ID NO: 68), CGNKRTRGC (SEQ ID NO: 69), VKVKVKVKVDPPTKVEVKVKV (SEQ ID NO: 70), LRKKLGKA (SEQ ID NO: 71), VVVVVVKK (SEQ ID NO: 72), AEAKAEAKAEAKAEAK (SEQ ID NO: 47), AEAKAEAK (SEQ ID NO: 73), AEAEAEAEAKAK (SEQ ID NO: 74), AEAEAKAK (SEQ ID NO: 75), AEAEAKAKAEAEAKAK (SEQ ID NO: 48), RADARADARADARADA (SEQ ID NO: 50), RADARGDARADARGDA (SEQ ID NO: 76), RADARADA (SEQ ID NO: 77), RARADADARARADADA (SEQ ID NO: 51), RARADADA (SEQ ID NO: 78), RARARARADADADADA (SEQ ID NO: 79), ADADADADARARARAR (SEQ ID NO: 80), DADADADARARARARA (SEQ ID NO: 81), RAEARAEARAEARAEA (SEQ ID NO: 82), RAEARAEA (SEQ ID NO: 83), KAKAKAKAEAEAEAEA (SEQ ID NO: 84), AEAEAEAEAKAKAKAK (SEQ ID NO: 49), KADAKADAKADAKADA (SEQ ID NO: 85), KADAKADA (SEQ ID NO: 86), AEAEAHAHAEAEAHAHA (SEQ ID NO: 87), AEAEAHAHA (SEQ ID NO: 88), HEHEHKHKHEHEHKHK (SEQ ID NO: 89), HEHEHKHK (SEQ ID NO: 90), FEFEFKFKFEFEFKFK (SEQ ID NO: 91), FEFKFEFK (SEQ ID NO: 92), LELELKLKLELELKLK (SEQ ID NO: 93), LELELKLK (SEQ ID NO: 94), KFDLKKDLKLDL (SEQ ID NO: 95), FKFEFKFF (SEQ ID NO: 96), FEFEFKFK (SEQ ID NO: 97), and RFRFRFRFRFRFRFRFRFRF (SEQ ID NO: 98). In such embodiments, a plurality of the PC-peptide conjugates may assemble into a nanofiber, nanotube, hydrogel, micelle, vesicle, nanoparticle, or suspension.

In some embodiments, the self-assembling polypeptide includes a modification to the C-terminus, to the N-terminus, or to both the C-terminus and N-terminus. N-terminal modifications may include, for example biotin and acetyl. C-terminal modifications may include, for example, amide. In some embodiments, the N-terminus is acetylated (which may be indicated by “Ac” for example). In some embodiments, the C-terminus is amidated (which may be indicated by “NH₂” for example).

The peptides described herein can be chemically synthesized using standard chemical synthesis techniques. In some embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides described herein. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill. In some embodiments, the self-assembling peptide is synthesized by a solid phase peptide synthesis.

b. Phosphoryicholine (PC)

The phosphoryicholine (PC)-conjugate peptide includes at least one PC molecule. Phosphorylcholine (PC) may also be referred to herein as “phosphocholine” or “choline phosphate.” PC is an intermediate in the synthesis of phosphatidylcholine in tissues. PC is made in a reaction, catalyzed by choline kinase, that converts ATP and choline into phosphocholine and ADP. PC may be found, for example, in lecithin. PC molecules include, for example, C₅H₁₄NO₄P and C₁₁H₂₂NO₆P. See TABLE 3. In some embodiments, the peptide fibril is coupled to a plurality of PC molecules. PC molecules may be obtained commercially or synthesized and purified by any suitable method known in the art.

TABLE 3
Exemplary PC molecules.

| | |
|---|---|
| 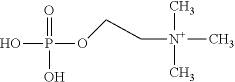 | C₅H₁₄NO₄P |
| 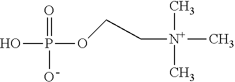 | |
| 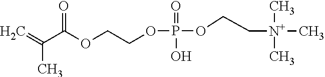 [SMILES: C=C(C)C(=O)OCCOP(=O)(O)OCC[N+](C)(C)C] | C₁₁H₂₂NO₆P |
| 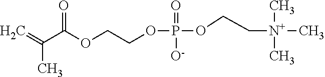 [SMILES: C=C(C)C(=O)OCCOP(=O)(O)OCC[N+](C)(C)C] | |

The PC molecule may be conjugated or coupled to a self-assembling peptide by any means known in the art, including, for example, click chemistry, Spytag/Spycatcher, oxime ligation, condensation reactions. In some embodiments, the PC molecule is covalently coupled to the self-assembling peptide. In some embodiments, the PC molecule is attached to the self-assembling peptide through a thiol reactive group. For example, the PC molecule may be attached to the self-assembling peptide through a thiol reactive group of a cysteine residue in a linker, further detailed below. The PC molecule may be attached to the self-assembling peptide via an asparagine residue in a linker. The PC molecule may be covalently coupled to a terminus of the self-assembling peptide. At least one PC molecule may be attached to the C-terminus or the N-terminus of the self-assembling peptide. In some embodiments, the PC molecule is covalently coupled to the N-terminus of the self-assembling peptide. In some embodiments, the PC molecule is covalently coupled to the C-terminus or C-terminal end of the self-assembling peptide. The conjugation of the PC molecule to the N-terminus or the N-terminal end of the self-assembling peptide may orient the PC molecule towards the exterior of the helical peptide fibril once a plurality of PC-conjugate peptides assembles into a nanofiber. In some embodiments, the PC molecules are exposed on the exterior surface of the nanofiber. In some embodiments, the PC molecules are exposed on the exterior surface of the helical filament of the nanofiber.

The phosphorylcholine (PC)-conjugate peptide may include at least one PC molecule. The phosphorylcholine (PC)-conjugate peptide may include 1 to 10 PC molecules attached to a self-assembling peptide. The phosphorylcholine (PC)-conjugate peptide may include 1, 2, 3, 4, 5, 6, 7, 8, 9, or 10 PC molecules attached to a self-assembling peptide. In embodiments including more than one PC molecule attached to a self-assembling peptide, all PC molecules may be attached to the same end of the self-assembling peptide. For example, 1, 2, 3, or 4 PC molecules together may be attached to the N-terminal end or to the C-terminal end of the self-assembling peptide. Once assembled into a nanofiber, the nanofiber may include n PC molecules, wherein n is an integer from 1 to 1,000, or 1 to 5,000, or 1 to 10,000, or 1 to 50,000, or 1 to 100,000.

In some embodiments, the antigens are exposed on the surface of the peptide fibril. In certain aspects the ratio of antigen to self-assembling peptide is 1:1000, 1:100: 1:10, or 1:1, including all values and ranges there between.

c. Linker

The phosphorylcholine (PC)-conjugate peptide may further comprise a linker. The linker may be between the PC molecule and the self-assembling peptide. The linker may be between the at least one PC molecule and the self-assembling peptide. In some embodiments, a linker is covalently attached to the self-assembling peptide between the PC molecule and the self-assembling peptide. In some embodiments, the linker comprises at least one cysteine. The at least one cysteine may be at the N-terminus of the linker. In some embodiments, the linker comprises one cysteine for each PC molecule attached to the self-assembling peptide. In some embodiments, the linker comprises at least one asparagine. The at least one asparagine may be at the N-terminus of the linker. In some embodiments, the linker comprises one asparagine for each PC molecule attached to the self-assembling peptide. In some embodiments, the linker comprises glycine and serine. In some embodiments, the linker comprises glycine and serine and cysteine. In some embodiments, the PC molecule is attached to the self-assembling peptide through a thiol reactive group in the linker. A PC molecule may be attached to the linker via a cysteine residue. A PC molecule may be attached to the linker via an asparagine residue.

In some embodiments, the conjugate peptide includes more than one linker. In such embodiments, the linkers may be the same or different from one another. The conjugate peptide may include at least 1, at least 2, at least 3, at least 4, at least 5, at least 6, at least 7, at least 8, at least 9, or at least 10 linkers. The conjugate peptide may include less than 20, less than 15, less than 10, or less than 5 linkers. The conjugate peptide may include between 1 and 20, between 5 and 15, or between 1 and 5 linkers. The linker may be positioned at the C-terminus of the self-assembling peptide, at the N-terminus of the self-assembling peptide, or at both the N- and C-termini of the self-assembling peptide. In some embodiments, the linker is positioned at the N-terminus of the self-assembling peptide. Multiple linkers may be positioned adjacent to one another.

The linker may comprise, for example, an oligoethylene glycol, polyethylene glycol, or an amino acid sequence selected from SEQ ID NO: 25 ((Ser-Gly)₂), SEQ ID NO: 26 (CCCCSGSG), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), SEQ ID NO: 29 (EAAAK)₂, and SEQ ID NO: 30 (GGAAY). In some embodiments, the linker comprises (Ser-Gly)₂ (SEQ ID NO: 25).

The PC-conjugate peptide may comprise (PC molecule)₄-CCCCSGSG-QQKFQFQFEQQ-NH₂ (SEQ ID NO: 34). Each one of the four PC molecules may be attached to a different Cys in the linker, for example, in the linker CCCCSGSG (SEQ ID NO: 26). The PC-conjugate peptide may comprise C₅H₁₄NO₄P-SGSG-QQKFQFQFEQQ-NH₂ (pa-PC-Q11, SEQ ID NO: 32). The PC-conjugate peptide may comprise C₁₁H₂₂NO₆P-CSGSG-QQKFQFQFEQQ-NH₂ (PC-Q11, SEQ ID NO: 33). The PC-conjugate peptide may comprise (C₁₁H₂₂NO₆P)ₙ-CCCCSGSG-QQKFQFQFEQQ-NH₂ (PC_{M}-Q11, SEQ ID NO: 34), wherein n is an integer selected from 1, 2, 3, 4, 5, 6, 7, 8, 9, and 10.

d. PAS Peptide

The phosphorylcholine (PC)-conjugate peptide may further comprise a PAS peptide. The PAS peptide may be muco-inert. The PAS peptide may enable or facilitate oral availability. The PAS peptide may comprise the amino acid sequence of ASPAAPAPASPAAPAPSAPA (SEQ ID NO: 35), or a peptide having at least 80%, 85%, 90%, or 95% identity thereto. In some embodiments, the PAS peptide comprises ASPAAPAPASPAAPAPSAPA (SEQ ID NO: 35). In some embodiments, the PAS peptide comprises H₂N-ASPAAPAPASPAAPAPSAPA-NH₂ (SEQ ID NO: 36). The PAS peptide may be conjugated to the self-assembling peptide at the opposite terminus from wherein the PC molecule is conjugated. The PAS peptide may be conjugated to the self-assembling peptide via a linker, as detailed above.

### 3. PADRE-Conjugate Peptide

Further provided herein is a PADRE-conjugate peptide. The PADRE-conjugate peptide may include a self-assembling peptide and at least one PADRE molecule attached thereto. The self-assembling peptide of the PADRE-conjugate peptide may be as detailed above for the PC-conjugate peptide. The PADRE molecule may comprise a polypeptide having the amino acid sequence of aKXVAAWTLKAa, wherein “X” comprises cyclohexylalanine and “a” comprises D-alanine (SEQ ID NO: 18). In some embodiments, the cyclohexylalanine comprises D-alanine.

In some embodiments, the PADRE-conjugate peptide further comprises a linker between the PADRE molecule and the self-assembling peptide. The linker of the PADRE-conjugate peptide may be as detailed above for the PC-conjugate peptide.

The PADRE-conjugate peptide may comprise, for example, the sequence of NH₂-aKXVAAWTLKAa-SGSG-QQKFQFQFEQQ-NH₂ (PADRE-Q11, SEQ ID NO: 37).

### 4. Plain Self-Assembling Peptides

Some embodiments include plain self-assembling peptides, which refers to a self-assembling peptide as detailed above, but without any antigen such as PADRE or a PC molecule attached thereto.

### 5. Nanofibers

Further described herein is a platform for vaccination or treatment based on peptides assembled into nanofibers. The nanofiber may also be referred to as a peptide fibril. The nanofibers may be comprised of alpha-helical peptides. The nanofibers may be comprised of beta-sheet peptides. In this strategy, peptides fold into a complex beta-sheet-based or alpha-helix-based nanofiber where individual peptide coils run perpendicular to the axis of a long fibril. Each self-assembling peptide may comprise or form an alpha helix. The plurality of self-assembling peptides may form a peptide fibril in the form of a helical filament. The resultant nanostructure is composed of thousands of individual peptides or more. The self-assembling peptide may be extended N-terminally with a flexible spacer and an immune epitope such as a PC molecule. In some embodiments, the nanofiber does not further comprise an adjuvant. In some embodiments, the nanofiber is an adjuvant.

Multiple epitope-bearing self-assembling peptides may be co-assembled into nanofibers composed of β-sheets or α-helices. Coiled coil folding requires more extensive design considerations compared to β-sheet fibrillization, as both inter-helical interactions as well as those between the C-terminus and the main chain must be considered.

The nanofiber may be comprised of 10 to 10,000 peptides. The nanofiber may include a combination of 10 to 10,000, or 100 to 10,000 peptides comprising PC-conjugate peptides and PADRE-conjugate peptides. The nanofiber comprises a combination of 10 to 10,000, or 100 to 10,000 peptides comprising PC-conjugate peptides, PADRE-conjugate peptides, and plain self-assembling peptides.

The nanofiber may include at least one PC-conjugate peptide. The nanofiber may include a plurality of PC-conjugate peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different PC-conjugate peptides. The PC-conjugate peptides making up a single nanofiber may be the same or different. The nanofiber may further include at least one PADRE-conjugate peptide. The nanofiber may include a plurality of PADRE-conjugate peptides. The nanofiber may include at least 1, 5, 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, or 5000 of the same or different PADRE-conjugate peptides. The PADRE-conjugate peptides making up a single nanofiber may be the same or different. In some embodiments, the nanofiber includes self-assembling peptides without PADRE or without a PC molecule conjugated thereto, which may be referred to as a plain self-assembling peptide. The nanofiber may further include at least one plain self-assembling peptide. The nanofiber may include a plurality of plain self-assembling peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different plain self-assembling peptides. The plain self-assembling peptides making up a single nanofiber may be the same or different.

A single nanofiber may include different peptides in a variety of ratios. The nanofiber may include a variety of ratios of PC-conjugate peptides to PADRE-conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are PC-conjugate peptides. In some embodiments, at least about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the peptides in the nanofiber are PADRE-conjugate peptides. In some embodiments, at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides. In some embodiments, about 50% of the peptides in the nanofiber are PC-conjugate peptides, about 2.5% of the peptides are PADRE-conjugate peptides, and about 47.5% of the peptides are plain self-assembling peptides. In some embodiments, the PC-peptide conjugate and the PADRE-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

The helical filament of the nanofiber may be formed around a central axis or core. The plurality of self-assembling peptides may form a peptide fibril in the form of a coiled coil. In some embodiments, the N-terminus of each self-assembling peptide is positioned at the exterior of the helical filament. The PC molecules may be exposed on the exterior surface of the nanofiber. The PADRE molecules may be exposed on the exterior surface of the nanofiber. An example of the self-assembling peptides formed into a peptide fibril is shown schematically in Egelman et al. (Structure 2015, 23, 280-289, incorporated herein by reference).

Nanofibers have been observed to be up to several microns long. The nanofiber can have a length of at least, at most, or exactly 100 nm, 200 nm, 300 nm, 400 nm, 500 nm, 600 nm, 700 nm, 800 nm, 900 nm, 1 μm, 1.2 μm, 1.4 μm, 1.6 μm, 1.8 μm, 2 μm, 2.2 μm, 2.4 μm, 2.6 μm, 2.8 μm, 3 μm, 3.2 μm, 3.4 μm, 3.6 μm, 3.8 μm, 4 μm, 4.2 μm, 4.4 μm, 4.6 μm, 4.8 μm, or 5 μm. The nanofiber may be about 100 nm to 1 μm, 100 nm to 2 μm, 100 nm to 3 μm, 100 nm to 4 μm, or 100 nm to 5 μm in length. The nanofiber can have a length of at least, at most, or exactly 0.01, 0.05, 0.1, 0.15, 0.20, 0.25, 0.5, 1, 5, 10, 20, 30, 40, 50, 75, 100, 125, 150, 175, 200, 225, 250, or 300 μm, including all values and ranges there between. In some embodiments, the nanofiber is at least 100, 150, 200, 250, 300, or 350 nanometers in length. In some embodiments, the nanofiber is less than 10, 5, or 2 μm in length. In some embodiments, the nanofiber is 50 nm to 600 nm in length. In certain aspects, the nanofiber has a molecular weight of at least 100, 500, 1,000, 5,000, 10,000, 100,000 Da to 1×10⁶, 1×10⁷, 7×10⁸ Da, including all values and ranges there between. The nanofiber can have a diameter or width of at least, at most, or exactly 5, 10, 15, or 20 nm. In some embodiments, the nanofiber is 5-20 nm in diameter or width.

### 6. Immune Response and Immunoassays

As discussed above, the compositions and methods provided herein include evoking or inducing an immune response in a subject against an antigen. The antigen may comprise a PC molecule. In one embodiment, the immune response can protect against or treat a subject having, suspected of having, or at risk of developing an infection or related disease, or a pathological condition such as inflammation or cancer or autoimmunity. One use of the immunogenic compositions is to provide effective vaccines. The compositions detailed herein may induce an immune response. The immune response may be an antigen-specific immune response. In some embodiments, the antigen-specific immune response is temporary or not life-long. The anti-PC antibodies generated may be IgM, or IgG, or IgA, or a combination thereof. In some embodiments, the antibodies are IgM antibodies. The immune response may include IgG antibody isotypes and subclasses. In some embodiments, the immune response comprises IgG1, IgG2, IgG3, or IgG4 antibody isotypes, or a combination thereof. In some embodiments, the immune response comprises IgM antibody isotypes. The immunogenic composition may have increased immunogenicity relative to a control. In some embodiments, the control comprises a PC molecule or other antigen without a self-assembling peptide.

The compositions and methods detailed herein may increase the level of anti-PC antibodies relative to a control. The level of anti-PC antibodies may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-PC antibodies may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-PC antibodies may be increased by about 5-95%, 10-90%, 15-85%, 20-80%, or 1.5-fold to 10-fold, relative to a control.

Further provided herein is the implementation of serological assays to evaluate whether and to what extent an immune response is induced or evoked by compositions. There are many types of immunoassays that can be implemented. Immunoassays include, but are not limited to, those described in U.S. Pat. No. 4,367,110 (double monoclonal antibody sandwich assay) and U.S. Pat. No. 4,452,901 (western blot), which are incorporated herein by reference. Other assays include immunoprecipitation of labeled ligands and immunocytochemistry, both in vitro and in vivo.

Immunoassays generally are binding assays. Certain immunoassays are the various types of enzyme linked immunosorbent assays (ELISAs) and radioimmunoassays (RIA) known in the art. Immunohistochemical detection using tissue sections is also useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

a. Protective Immunity

In some embodiments, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” may refer to the development of a humoral (antibody mediated), cellular (mediated by antigen-specific T cells or their secretion products) or both humoral and cellular response directed against a protein, peptide, carbohydrate, or polypeptide in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to Staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition as detailed herein can be administered to a subject who then acts as a source or donor for globulin, produced in response to challenge with the antigenic composition (“hyperimmune globulin”), that contains antibodies directed against Staphylococcus or other organism, for example. A subject thus treated would donate plasma from which hyperimmune globulin would then be obtained, via conventional plasma-fractionation methodology, and administered to another subject in order to impart resistance against or to treat Staphylococcus infection. Hyperimmune globulins are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed from small molecules. For example, B-cell epitopes may be formed from a PC molecule. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM, and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific. Monoclonal antibodies can be produced by hyperimmunization of an appropriate donor with the antigen or ex-vivo by use of primary cultures of splenic cells or cell lines derived from spleen (Anavi, 1998; Huston et al., 1991; Johnson et al., 1991; Mernaugh et al., 1995, each incorporated herein by reference).

As used herein, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

### 7. Pharmaceutical Compositions

Further provided herein are pharmaceutical compositions comprising the PC-peptide conjugate or nanofibers detailed herein. Compositions can include a peptide fibril coupled to a plurality of antigens such as PC molecule, and may be referred to as a “fibril complex.” In some embodiments, the composition does not further comprise an adjuvant. In some embodiments, the composition further comprises an adjuvant. In some embodiments, the peptide fibril is an adjuvant.

The preparation of pharmaceutical compositions such as vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such pharmaceutical compositions are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, pharmaceutical compositions are formulated with a combination of substances, as described in U.S. Pat. Nos. 6,793,923 and 6,733,754, which are incorporated herein by reference.

Pharmaceutical compositions may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The compositions described herein may be formulated into a pharmaceutical composition as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, compositions are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the vaccine will depend on the route of administration and will vary according to the size and health of the subject.

The compositions and related methods, particularly administration of a peptide conjugate or nanofiber, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a peptide conjugate or nanofiber or pharmaceutical composition is used in conjunction with an additional treatment described herein. Alternatively, the therapy may precede or follow the other agent treatment by intervals ranging from minutes to weeks. In embodiments where the other agents and/or a proteins is administered separately, one may generally ensure that a significant period of time did not expire between the time of each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6, or 7) to several weeks (1, 2, 3, 4, 5, 6, 7, or 8) lapse between the respective administrations.

Various combinations may be employed, for example antibiotic therapy is “A” and the immunogenic composition is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the pharmaceutical compositions to a patient/subject will follow general protocols for the administration of such compounds, taking into account the toxicity, if any. It is expected that the treatment cycles would be repeated as necessary. It also is contemplated that various standard therapies, such as hydration, may be applied in combination with the described therapy.

In some embodiments, pharmaceutical compositions are administered to a subject. Different aspects involve administering an effective amount of a composition to a subject. In some embodiments, immunogenic compositions may be administered to the patient to protect against infection by one or more microbial pathogens. Additionally, such compounds can be administered in combination with an antibiotic or other known anti-microbial therapy. Such compositions will generally be dissolved or dispersed in a pharmaceutically acceptable carrier or aqueous medium. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration of the compositions will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. In some embodiments, the composition is administered to the subject intravenously, intraarterially, intraperitoneally, subcutaneously, intranasally, intramuscularly, or intratumorally. In some embodiments, the immunogenic composition is administered orally.

For parenteral administration in an aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions may be administered in a manner compatible with the dosage formulation and in such amount as is therapeutically or prophylactically effective. The formulations may be easily administered in a variety of dosage forms, such as the type of injectable solutions described above.

### 8. Methods

a. Methods of Inducing an Immune Response

Further provided herein are methods of inducing an immune response in a subject. The methods may include administering to the subject a PC-conjugate peptide, or nanofiber, or pharmaceutical composition as detailed herein. In some embodiments, the B1a cells in the subject express antibodies upon or subsequent to administration. The antibodies may be selected from IgM, IgG, and IgA, or a combination thereof. The IgG antibodies may comprise IgG1, IgG2, IgG3, or IgG4, or a combination thereof. In some embodiments, the antibodies are IgM antibodies. Further provided herein is an antibody produced in the immune response. In some embodiments, the PC-peptide conjugate or the nanofiber is administered to the subject intraperitoneally, orally, sublingually, intravenously, nasally, buccally, transdermally, intranasally, or intramuscularly, or via the lung. In some embodiments, the PC-peptide conjugate or the nanofiber is administered to the subject intraperitoneally or orally. In some embodiments, method further comprises administering at least one additional therapeutic agent. The at least one additional therapeutic agent may be administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition. The at least one additional therapeutic agent may comprise an adjuvant. In some embodiments, the adjuvant comprises CpG, cholera toxin B subunit (CTB), STING agonists like cyclic dinucleotides, alum, and/or MF59.

b. Methods of Stimulating B1a Cells to Express Antibodies

Further provided herein are methods of stimulating B1a cells to express antibodies. The methods may include administering to a subject a PC-conjugate peptide, or nanofiber, or pharmaceutical composition as detailed herein. In some embodiments, anti-PC antibodies are produced in the subject. The antibodies may be selected from IgM, IgG, and IgA, or a combination thereof. The IgG antibodies may comprise IgG1, IgG2, IgG3, or IgG4, or a combination thereof. In some embodiments, the antibodies are IgM antibodies. In some embodiments, the PC-peptide conjugate or the nanofiber is administered to the subject intraperitoneally, orally, sublingually, intravenously, nasally, buccally, transdermally, intranasally, or intramuscularly, or via the lung. In some embodiments, method further comprises administering at least one additional therapeutic agent. The at least one additional therapeutic agent may be administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition. The at least one additional therapeutic agent may comprise an adjuvant. In some embodiments, the adjuvant comprises CpG, cholera toxin B subunit (CTB), STING agonists like cyclic dinucleotides, alum, and/or MF59.

c. Methods of Treating an Inflammatory Disease or Disorder

Further provided herein are methods of treating an inflammatory disease or disorder in a subject. The methods may include administering to a subject a PC-conjugate peptide, or nanofiber, or pharmaceutical composition as detailed herein. In some embodiments, the PC-peptide conjugate or the nanofiber is administered to the subject intraperitoneally, orally, sublingually, intravenously, nasally, buccally, transdermally, intranasally, or intramuscularly, or via the lung. In some embodiments, method further comprises administering at least one additional therapeutic agent. The at least one additional therapeutic agent may be administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition. The at least one additional therapeutic agent may comprise an adjuvant. In some embodiments, the adjuvant comprises CpG, cholera toxin B subunit (CTB), STING agonists like cyclic dinucleotides, alum, and/or MF59.

In some embodiments, the inflammatory disease or disorder includes inflammatory bowel disease (IBD), ulcerative colitis, Crohn's disease, rheumatoid arthritis, cirrhosis, atherosclerosis, cardiovascular inflammation, or psoriasis, or a combination thereof.

Embodiments can be used to treat or ameliorate a number of immune-mediated, inflammatory, autoimmune, or autoimmune-inflammatory diseases, e.g., allergies, asthma, diabetes (e.g. type 1 diabetes), graft rejection, etc. Examples of such diseases or disorders also include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, Pemphigus (including Pemphigus vulgaris, Pemphigus foliaceus, Pemphigus mucus-membrane pemphigoid, and Pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolysis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

d. Methods of Altering Gut Microbiome Activity

Further provided herein are methods of altering gut microbiome activity. The methods may include administering to a subject a PC-conjugate peptide, or nanofiber, or pharmaceutical composition as detailed herein. In some embodiments, the PC-peptide conjugate or the nanofiber is administered to the subject intraperitoneally orally, sublingually, intravenously, nasally, buccally, transdermally, intranasally, or intramuscularly, or via the lung. Upon or after administration, bacterial diversity in the colon may be reduced. Bacteria in the gut may include, for example, Campylobacter, Salmonella, Shigella, Escherichia, and Yersinia species. There may be damage to the colon during colitis, for example. During colitis, the colon may be “leaky” and release bacteria to the spleen. The compositions detailed herein may result in less bacteria spreading to the spleen. The compositions detailed herein may reduce ulceration in the colon. In some embodiments, method further comprises administering at least one additional therapeutic agent. The at least one additional therapeutic agent may be administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition. The at least one additional therapeutic agent may comprise an adjuvant. In some embodiments, the adjuvant comprises CpG oligodeoxynucleotides, or cholera toxin B subunit (CTB), or STING agonist, or cyclic dinucleotides, or alum, or MF59, or a combination thereof.

### 9. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

Peptide Synthesis. Peptide sequences are listed in TABLE 1. All peptides were synthesized using standard Fmoc solid-phase synthesis. To create fluorescent peptides, 5 (6)-TAMRA (AnaSpec, AS-81120-1) was conjugated to the N-terminus with the coupling reagents CI-HOBt and N,N′-Diisopropylcarbodiimide (DIC). PC conjugation to the N-terminus of peptides to generate pa-PC-Q11 was achieved by activating phosphocholine chloride calcium salt tetrahydrate (Sigma, P0378) with EDC and NHS for 1 hour and then reacting it overnight with resin-bound peptide in DMF while heating to 60° C. PC conjugation to thiols to form PC-Q11 and PC_{M}-Q11 was accomplished by first selectively deprotecting the thiols of Fmoc-Cys(4-methoxytrityl)-OH (Sigma, 727997) on resin-bound peptide in 2% TFA in DCM. Then, 2-methacryloyloxyethyl phosphorylcholine (Sigma, 730114) was mixed with resin-bound peptide in DMF overnight. Peptides were cleaved in a 95%/2.5%/2.5% TFA/triisopropylsilane/water cocktail and were purified via reverse-phase HPLC. Finally, peptide molecular weight was confirmed using MALDI-TOF mass spectrometry on a Bruker Autoflex Speed LRF MADLI-TOF with a α-cyano-4-hydroxycinnamic acid matrix.

TABLE 1
Peptides investigated.

| Peptide | Sequence |
|---|---|
| Q11 | AC-QQKFQFQFEQQ-NH₂ |
| | (SEQ ID NO: 99) |
| TAMRA-Q11 | 5-(and-6)-carboxytetramethylrhodamine- |
| | SGSG-QQKFQFQFEQQ-NH₂ (SEQ ID NO: 37) |
| PADRE-Q11 | NH₂-aKXVAAWTLKAa-SGSG-QQKFQFQFEQQ-NH₂ |
| | X = cyclohexylalanine, a = D-alanine |
| | (SEQ ID NO: 37) |
| Cys₄-Q11 | CCCCSGSG-QQKFQFQFEQQ-NH₂ |
| | (SEQ ID NO: 34) |
| pa-PC-Q11 | C₅H₁₄NO₄P-SGSG-QQKFQFQFEQQ-NH₂ |
| | (SEQ ID NO: 32) |
| PC-Q11 | C₁₁H₂₂NO₆P-CSGSG-QQKFQFQFEQQ-NH₂ |
| | (SEQ ID NO: 33) |
| PC_{M}-Q11 | (C₁₁H₂₂NO₆P)ₙ-CCCCSGSG-QQKFQFQFEQQ-NH₂ |
| | n = 1-4 (SEQ ID NO: 34) |

N-terminal Ac indicates acetylation; C-terminal NH₂ indicates amidation.

Nanofiber Preparation. Nanofibers were prepared by weighing out lyophilized peptide components and mixing as dry powders before dissolving in sterile water at 8 mM and incubating at 4° C. overnight. The next day, the solutions were diluted to 2 mM total peptide in sterile 1×PBS (diluted from 10×PBS, 45001-130, VWR) via the addition of water and 10×PBS, at which point nanofiber assembly begins. For formulations with CpG (ODN 1826, IAX-200-002-3001, Innaxon), 10 μg CpG in water was included. Nanofibers were allowed to assemble for at least 3 hours at room temperature prior to use. For pa-PC-Q11 and PC-Q11 formulations, 1 mM pa-PC-Q11 or PC-Q11 was assembled with 1 mM Q11. PC_{M}-Q11 formulations contained 0.2 mM PC_{M}-Q11 with 1.8 mM Q11. In cases where the T-cell epitope, PADRE, was included, 0.05 mM PADRE-Q11 was substituted for 0.05 mM of the Q11 backbone. To make fluorescent nanofibers, 0.2 mM TAMRA-Q11 was substituted for 0.2 mM of the Q11 component. To make nanofibers with extra cysteines, 0.2 mM Cys₄-Q11 was substituted for 0.2 mM of the Q11 component.

Transmission Electron Microscopy (TEM). Formed nanofibers were visualized on TEM by diluting to 0.2 mM in 1×PBS and placing 5 μL on Formvar/Carbon 300 mesh copper grids (Electron Microscopy Sciences, FCF300-CU). The samples were then washed with water, and nanofibers were negatively stained with 1% wild type/volume uranyl acetate. The grids were then dried and imaged using an FEI Tecnai G² Twin TEM. Nanofiber widths from TEM images were measured using ImageJ.

Thioflavin T (ThT) Assay. β-sheets were detected by Thioflavin T (ThT) (Alfa Aesar, J61043) binding and resulting fluorescence. 180 μL of 0.05 mM ThT was added to 20 μL of 2 mM nanofibers in a 96-well black plate and allowed to incubate for 1 min. Fluorescence intensity was then determined using an excitation wavelength of 440 nm and emission wavelengths from 460-600 nm at 5 nm intervals. A peak fluorescence intensity at an emission wavelength around 488 nm is indicative of β-sheets.

Zeta Potential. Nanofibers were diluted to 0.2 mM in 1×PBS and zeta potential measurements were conducted at 25° C. using an Anton Paar Litesizer 500.

ELISA for Measuring Nanofiber Epitope Reactivity. The reactivity of PC conjugation methods was determined via binding of a mouse anti-PC IgM monoclonal antibody (Clone: BH8) (Sigma, MABF2084) to nanofibers. Nanofibers were diluted to 0.2 mM and coated onto 96-well plates at 4° C. overnight. PC-BSA (Biosearch Technologies, PC-1011-10) was coated at 20 μg/mL in 1×PBS overnight as a positive control, and PBS was used as a negative control. The next day, the plates were washed using 1×PBS with 0.05% Tween 20 (1×PBST) and then blocked with Superblock blocking buffer solution (Thermo Fisher, 37515) three times. This was followed by the addition anti-PC IgM which was serially diluted in 1% BSA in 1×PBST by 5-fold steps beginning from 1:100 (5 μg/mL) and allowed to incubate for 2 h at room temperature. Anti-PC IgM bound to nanofibers was then detected with HRP-conjugated goat anti-mouse IgM (Southern Biotech, 1021-05) and the absorbance values at 450 nm were measured.

Mice and Immunizations. Female C57BL/6 mice (Envigo) age 6-10 weeks were used to initiate experiments except for the Il10^{-/-} colitis study where female B6.129P2-Il10^{tm1Cgn}/J (The Jackson Laboratory) mice age 5-7 weeks were used. While female mice were used for most experiments, age-matched male C57BL/6 mice (Envigo) were used in a one cycle acute DSS colitis model to confirm efficacy across sexes. Mice were housed at Duke University. All experimental procedures were performed on protocols (A264-18-11 and A199-21-09) approved by the Duke University Institutional Animal Care and Use Committee (IACUC). For subcutaneous immunizations, anesthetized mice were injected once behind each shoulder with 50 μL of 2 mM peptide nanofiber solution or 1×PBS. For intraperitoneal immunizations, mice were injected with 100 μL of 2 mM peptide nanofiber solution or 1×PBS. Nanofiber immunizations with adjuvant contained 10 μg CpG (ODN 1826, IAX-200-002-3001, Innaxon). For injections of only CpG, 10 μg of CpG in 100 μL 1×PBS was used. Mice were typically given three immunizations, with the specific boosting schedules stated in each figure caption, usually at weeks 0, 2, and 4, as this was determined to produce robust antibody responses in a short time frame.

Nanofiber Uptake and Evaluation. The cell types present and their propensity to uptake nanofibers in the i.p. space was tested by injecting mice i.p. with 100 μL of 2 mM TAMRA-labeled fluorescent nanofibers. 4 hours later, mice were euthanized and i.p. lavage was conducted via the injection of 2 mL of cold 1×PBS into the peritoneal cavity and massaging the abdomen for 30 seconds. The solution was then carefully extracted from the i.p. space and the cells were isolated followed by rinsing with ice cold flow buffer (2% heat-inactivated FBS in 1×PBS). The cells present were treated with mouse BD Fc Block (Clone: 2.4G2) (BD Biosciences, 553142) in flow buffer on ice for 30 min and then washed and stained with antibodies against the following markers: CD5-APC (Clone: 53-7.3) (BioLegend, 100626), F4/80-PerCP/Cy5.5 (Clone: BM8) (BioLegend, 123128), CD19-APC/Cy7 (Clone: 6D5) (BioLegend, 115530), CD11c-PE/Cy7 (Clone: N418) (BioLegend, 117318), and CD86-BV605 (Clone: GL1) (BD Biosciences, 2272591) in flow buffer for 30 min on ice. TAMRA-fluorescence as an indication of nanofiber uptake was measured on the PE channel. Cells were then washed in flow buffer and stained with DAPI. Flow cytometry was conducted on a BD FACSCanto II cytometer and the results were analyzed using FlowJo software.

Detection of Anti-PC Antibody Responses via ELISA. Anti-PC antibody responses in serum collected from the submandibular vein in mice were measured using ELISA. Briefly, plates were coated with 20 μg/mL PC-BSA (Biosearch Technologies, PC-1011-10) in 1×PBS or just 1×PBS at 4° C. overnight. The next day, the plates were washed using 1×PBST (1×PBS with 0.05% Tween 20) and then blocked three times with Superblock blocking buffer solution (Thermo Fisher, 37515). Serum diluted from 1:10² to 1:10⁵ in 1% BSA in 1×PBST was the added to the plate and allowed to incubate for 2 h at room temperature. Anti-PC IgG was detected using HRP-conjugated Fcγ fragment-specific goat anti-mouse IgG (Jackson Immuno Research, 15-035-071). IgG subclasses and IgM anti-PC antibodies were detected using Southern Biotech HRP-conjugated goat anti-mouse antibodies (IgG1, IgG2b, IgG2c, IgG3, & IgM: 5300-05B). Area under the curve (AUC) measurements were calculated using background-subtracted absorbance values at dilutions from 1:10² to 1:10⁵. Antibody titers were defined using an absorbance cutoff above background of 0.2 OD, where titer 2 represents an OD value greater than 0.2 at 1:10² dilution and so on up to titer 5 at 1:10⁵ dilution.

T Cell ELISpot. T-cell responses were measured using ELISpot. Mice were euthanized 6 days after booster immunization and splenocytes were isolated using Lympholyte-M (Cedarlane, c15031). Splenocytes were stimulated with either media (negative control), Concanavalin A (ConA) (Sigma, C5275) (positive control), or PADRE peptide (1 μM) and then plated at 250,000 cells/well on IL-4 and IFN-γ capture antibody-coated 96-well ELISpot plates (Millipore, MSIPS4510). IL-4 (BD, 551878) and IFNγ (BD, 551881) detection antibodies followed by streptavidin-alkaline phosphatase (Mabtech, 3310-0) and Sigmafast BCIP/NBT (Sigma, B5655) were used to develop cell spots. The quantification of cell spots was then outsourced to Zellnet Consulting Services which uses a Zeiss KS ELISPOT reader. ELISpot counts shown represent spot counts after the subtraction of spots in the negative control wells for each sample.

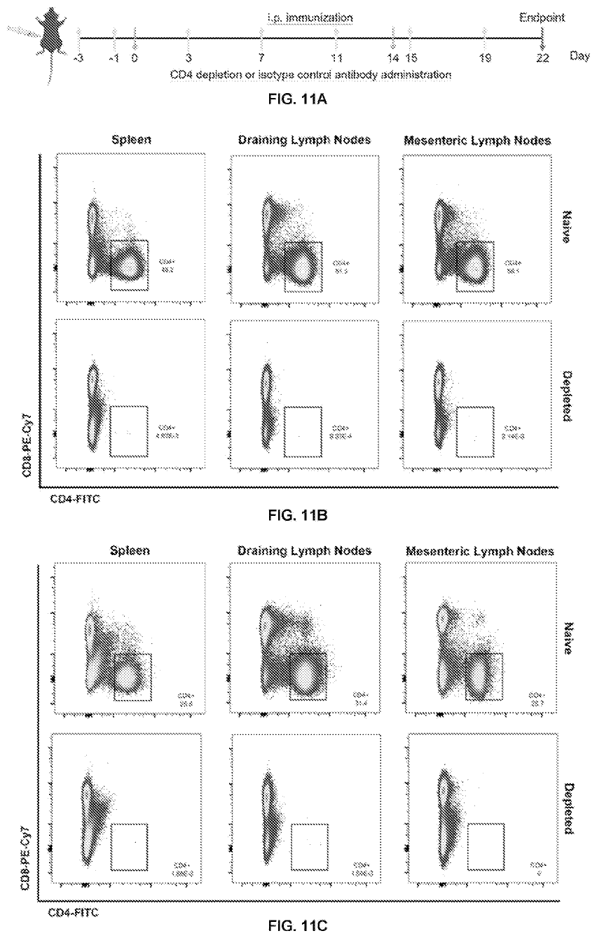

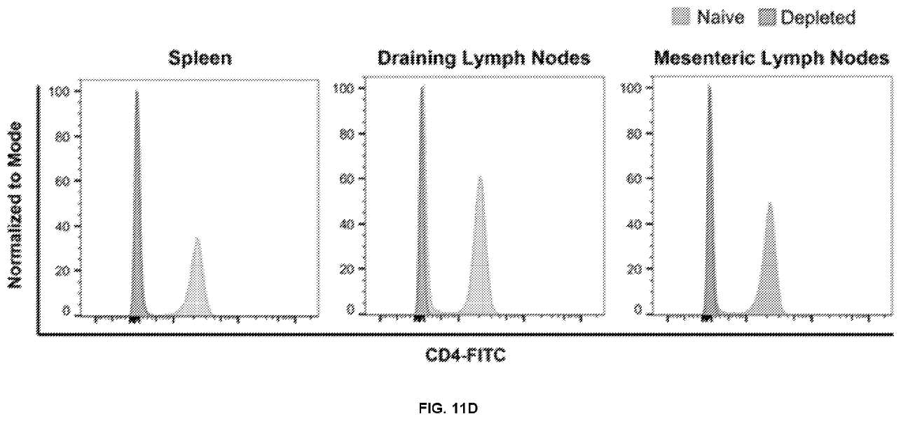

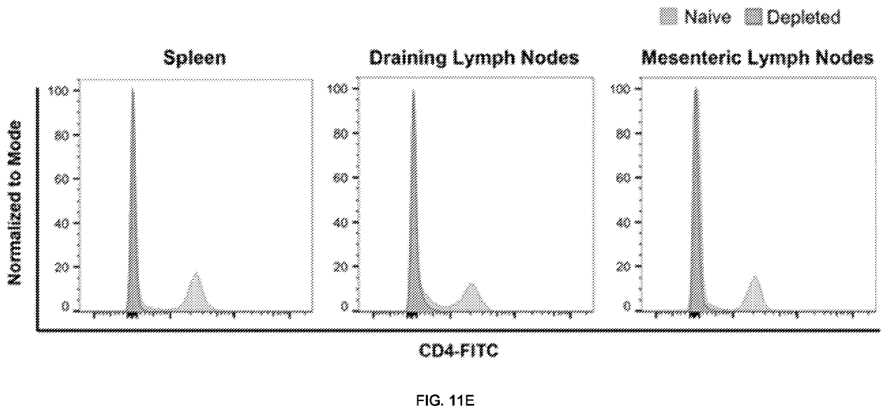

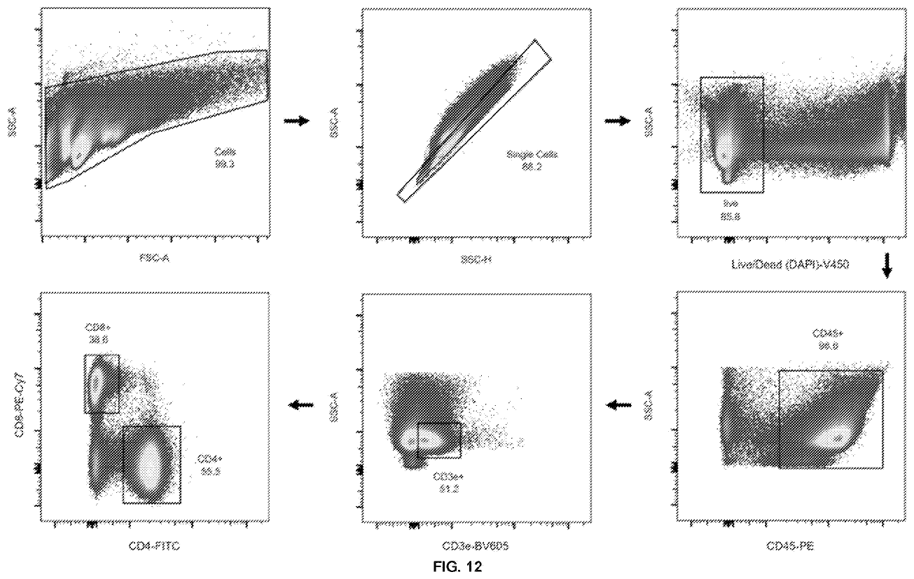

CD4 T cell Depletion. Mice were given 200 μg of either anti-mouse CD4 (InVivoMAb, clone GK1.5, BioXCell, BE0003-1) or isotype control (InVivoMAb rat IgG2b isotype control anti-KLH, clone LTF-2, BioXCell, BE0090) antibodies in 100 μL InVivoPure pH 6.5 dilution buffer (BioXCell, IP0065) via i.p. injection at days −3, −1, 3, 7, 11, 15, and 19. On days 0 and 22, cells from the spleen, draining lymph nodes (axial, brachial, and inguinal), and mesenteric lymph nodes of unimmunized mice were isolated via physical separation using a 70 μm filter in order to verify CD4⁺ T-cell depletion (FIGS. 11A-11E, representative flow cytometry shown in FIG. 12). The cells were treated with mouse BD Fc Block (Clone: 2.4G2) (BD Biosciences, 553142) in flow buffer on ice for 30 min and then washed and stained with antibodies against the following markers: CD4-FITC (Clone: GK1.5) (BD Biosciences, 557307), CD45-PE (Clone: 30-F11) (BioLegend, 103106), CD8a-PE-Cy7 (Clone: 53-6.7 (BD Biosciences, 552877), and CD3e-BV605 (Clone: 145-2C11) (BD Biosciences, 563004) in flow buffer for 30 min on ice. Cells were then washed in flow buffer and stained with DAPI. Flow cytometry was conducted on a BD FACSCanto II cytometer and the results were analyzed using FlowJo software. To determine the importance of PADRE T-cell epitope inclusion in immunizations, anti-CD4 or isotype control antibody-administered mice were immunized on days 0 and 14, with anti-PC antibodies measured at days 0, 7, and 22 via ELISA and T-cell responses measured on day 22 via ELISpot.

DSS-Induced Colitis and Assessment. Before inducing colitis, mice from each treatment group were divided amongst the cages to avoid any cage-related disease effects. This excluded the PBS without DSS (healthy) control mice which remained in their own cage and received normal drinking water for the duration of the experiment. Colitis was induced by administering 2% (w/v) colitis-grade dextran sodium sulfate (MP Biomedicals, 160110) in drinking water. For determination of prophylactic immunization efficacy, mice were immunized i.p. 3 times at days −35, −21 and −7 before colitis induction on Day 0. Chronic colitis was induced via DSS-containing water given for 5 days followed by 5 days of normal drinking water for 3 cycles, resulting in a 30-day model. A shorter, acute DSS-induced colitis model consisting of one cycle of colitis was achieved by giving 5 days of 2% DSS in water followed by 5 days of normal drinking water for a total of 10 days and was also examined in a preventative immunization scenario. Mice were weighed daily and evaluated for the IACUC approved humane endpoint of >30% weight loss as DSS mice regain weight quickly after DSS administration ceases. They were also monitored for occult blood with Hemoccult tape and developer (Beckman Coulter, 63202) and fecal consistency. Disease activity index (DAI) scores were a combination of weight loss (≥100%=0, 90-99%=1, 80-89%=2, >80%=3), occult blood (no blood=0, blood=3), and fecal consistency (hard=0, soft=1, softer=2, runny=3), with a possible daily maximum score of 9. If a mouse did not produce a fecal sample, its DAI score was excluded for that day. At the experimental endpoint, mice were euthanized, and colon lengths were measured from the base of the cecum to the rectum using digital calibrators. To determine immunization efficacy in a therapeutic colitis setting, colitis was first induced by administering one cycle of colitis achieved by 5 days of 2% DSS in water followed by 5 days of normal drinking water for a total of 10 days. Mice were monitored daily as described above until they on average regained their original body weight on day 20. At this point, i.p. immunizations were initiated and given three times over a 4-week period on days 20, 32, and 45, and colitis monitoring was reduced to twice weekly. An additional cycle of colitis was then induced on day 51 via 5 days of 2% DSS in water followed by 5 days of normal drinking water for a total of 10 days with daily monitoring. The entire therapeutic model ended on day 60, and colon lengths were measured as described above.

Il10^{-/+} Colitis and Assessment. Il10^{-/-} mice were injected i.p. with either PBS or PC_{M}-Q11/CpG at days −35, −21, and −7. Mice were acclimated to powdered rodent food and monitored to ensure their weight had stabilized for 10 days before colitis was triggered on day 0 via the administration of 200 ppm piroxicam (Sigma, P0847) in powdered rodent food for 7 days with free access to normal drinking water (Hale, L. P., et al. Inflamm. Bowel. Dis. 2005, 11, 1060-1069; Berg, D. J. et al. Gastroenterology 2002, 123, 1527-1542; incorporated herein by reference). Mice were monitored daily throughout piroxicam administration and for an additional 16 days after piroxicam cessation. Monitoring included weight loss, fecal consistency, and occult blood measurements. Fecal consistency scores were as follows: hard=0, soft=1, softer=2, runny=3. Hemoccult tape and developer (Beckman Coulter, 63202) was used to detect occult blood and scored as follows: no blood=0, trace=1, moderate=2, visible blood=3). At the experimental endpoint, mice were euthanized, and colons were prepared for histology and B cell flow cytometry.

FITC-dextran Measurements. On the last day of the chronic and therapeutic DSS colitis models, mice were fasted overnight and then given 0.6 mg/g 3-5 KDa m.w. FITC-dextran (Sigma, FD4) in 100 μL of sterile 1×PBS via oral gavage. Serum samples were collected 3 h later at the experimental endpoint. To determine the intestinal barrier permeability, serum samples were diluted in 1×PBS and FITC fluorescence intensity was measured at an excitation of 488 nm and emission of 520 nm. Values were converted to pg/mL via a standard curve of FITC-dextran in serum.

Spleen Bacteria Measurements. At the conclusion of the DSS experiments, spleens were harvested and weighed followed by homogenization in 500 μL of 0.1% Triton X-100 in sterile 1×PBS using a Tissue Tearer handheld homogenizer (Biospec Products). 25 μL of homogenate solution was then spread on each quarter of culture plates consisting of tryptic soy broth No. 2 (Sigma, 51228) and Difco agar, granulated (BD Biosciences, 214530) in dilutions from 100 to 103. After a 48 hr incubation at 37° C., colony forming units (CFUs) were counted and converted to CFU/g of spleen tissue via the equations:

$$\frac{CFU}{mL} = \frac{colony count \times dilution factor}{volume spread}$$

$$\frac{CFU}{g} = \frac{\frac{CFU}{mL} \times homogenate volume}{tissue mass}$$

Colon IL-10 Quantification. At the conclusion of some DSS experiments, one half of the harvested mouse colons were flushed with PBS to remove fecal matter and flash frozen in liquid nitrogen. Later, colons were thawed on ice, weighed, and homogenized in 2 μL/mg of Tissue Extraction Reagent I (Thermo Fisher, FNN0071) with a protease inhibitor tablet (Thermo Fisher, A32955) using a Navy Bead Lysis Kit (Next Advance, NAVYE1). The amount of IL-10 in colon homogenate supernatants was then measured using an IL-10 mouse ELISA kit (Invitrogen, 88-7105-22) in accordance with the manufacturer's protocol.

Determination of Colon B cell Populations. Small colon samples collected from either after immunization only or after colitis experiments were collected at experimental endpoints. Colon samples were flushed with PBS and then digested in a solution containing 200 U/mL collagenase from Clostridium histolyticum (Sigma, C5138) and 0.1 mg/mL DNase I (Sigma, 10104159001) in 1×PBS. Digestion occurred at 37° C. while shaking for a total of 30 minutes. The solutions were then filtered to isolate cells, spun down, and transferred into ice cold flow buffer (2% heat-inactivated FBS in 1×PBS). The cells were blocked with mouse BD Fc Block (Clone: 2.4G2) (BD Biosciences, 553142) for 30 min on ice and then washed in flow buffer and stained with antibodies against CD5-APC (Clone: 53-7.3) (BioLegend, 100626), CD19-APC/Cy7 (Clone: 6D5) (BioLegend, 115530), B220-PE (Clone: RA3-6B2) (BioLegend, 103208), and CD138-PE/Cy7 (Clone: 281-2) (BioLegend, 103208) in flow buffer for 30 min on ice. Cells were washed and DAPI was added. Flow cytometry was then conducted on a BD FACSCanto II cytometer and the results were analyzed using FlowJo software.

Colon Histology. At the end of the chronic and therapeutic DSS colitis experiments, proximal and distal colon samples were collected for histology. At the end of the Il10^{-/-} colitis experiment, the colon was divided into 5 sections for histology: cecum, proximal, middle, and distal colon, and rectum/terminal colon. Colon samples were fixed in Carnoy's solution (a 6:3:1 ration of absolute ethanol, chloroform, and glacial acetic acid) for 3 h before transfer to 100% absolute ethanol. Colon samples were then processed and embedded into paraffin blocks. Sections were stained with hematoxylin and eosin and then evaluated by a pathologist blinded to treatment according to the scoring system in TABLE 2 (Hale, L. P., et al. Inflamm. Bowel Dis. 2005, 11, 1060-1069; Burich, A. et al. Am. J. Physiol. Gastrointest. Liver Physiol. 2001, 281, G764-77; incorporated herein by reference). Ulceration measurements were conducted on whole slide digital images of H&E-stained slides by a pathologist blinded to treatment. The extent of each ulceration was outlined using the “pen tool” and its length in μm was calculated using ImageScope software (Leica). The total length of each intestinal segment was measured using the ImageScope “ruler tool”. The total surface length examined was generally at least twice the segment length since the tissues were embedded so that both sides of the segment could be evaluated. However, the total surface length examined within each segment, which was highly dependent on mucosal folding, was not calculated. Therefore, these measurements are denoted at μm ulcer/mm segment length as opposed to percent ulceration.

TABLE 2
Histological Scoring Parameters.

| | Score |
|---|---|
| Mucosa (M) | |
| No significant lesions | 0 |
| Mild, epithelial hyperplasia | 1 |
| Moderate epithelial hyperplasia | 2 |
| Severe epithelial hyperplasia, with crypt branching | 3 |
| or herniation | |
| Inflammation (I) | |
| None | 0 |
| Mild, limited to mucosa | 1 |
| Moderate, in mucosa and submucosa | 2 |
| Severe, with obliteration of normal architecture, | 3 |
| erosions, &/or crypt abscesses | |
| Level 3 changes plus ulceration | 4 |
| Extent (E) | |
| No significant changes | 0 |
| <5% of segment affected | 1 |
| 5-30% of segment affected | 2 |
| 31-60% of segment affected | 3 |
| >60% of segment affected | 4 |

E1 = % of segment affected in any manner
E2 = % of segment with level 3 or 4 changes
Segment score = M + I + E1 + E2 (maximum of 15)
For IL-10^{-/-} model: Total score is derived from summing segment scores for all segments examined (cecum, proximal, middle, and distal colon, rectum) (maximum of 75)

Microbiome Analysis. Fecal samples collected from mice in the chronic DSS model were processed for microbiome analysis by the Duke Microbiome Core Facility. DNA extraction was accomplished using a Qiagen DNeasy PowerSoil Pro Kit (Qiagen, 47014) and bacteria contained in the samples was identified by using polymerase chain reaction (PCR) to amplify the V4 region of the 16S rRNA gene with forward primer 515 (515F) and reverse primer 806 (806R) according to the Earth Microbiome Project (http://www.earthmicrobiome.org/), which allows for multiplexed sequencing. A Qubit dsDNA HS assay kit (Thermo Fisher, Q32854) used in conjunction with a Promega GloMax plate reader allowed for determination of the concentration of PCR products. The Duke Sequencing and Genomic Technologies shared resource conducted sequencing on pooled equimolar 16S rRNA PCR products using an Illumina MiSeq instrument set up for 250 base-pair paired-end sequencing runs. Sequencing data was then processed using QIIME 2 software (Bolyen, E. et al. Nat. Biotechnol. 2019, 37, 852-857, incorporated herein by reference) by first trimming the noise from the data and then generating a phylogenic diversity tree followed by taxonomic analysis at the family level. Beta diversity was then characterized via the creation of a Jaccard Emperor plot, and alpha diversity measures including observed operational taxonomic units (OTUs) and Shannon diversity indices were calculated.

Apoptotic Cell Binding Assay. Caco2 human colon epithelial cells (ATCC, HTB-37™) were seeded at 3×10⁴ cells per well in a 48-well plate in complete Caco2 media (DMEM+10% heat-inactivated FBS+4.5 g/L D-glucose+1× L-glutamine+1× sodium pyruvate+1× non-essential amino acids+1× penicillin-streptomycin). 24 hours later, cells were treated with 2% colitis-grade dextran sodium sulfate (MP Biomedicals, 160110) in complete Caco2 media. After 24 hours, the cells were then isolated and pre-treated with Fc-block (Human TruStain FcX, BioLegend, 422302) in flow buffer (2% heat-inactivated FBS in 1×PBS) for 10 min at room temperature. Cells were washed in flow buffer and then treated with either 1×PBS or pooled serum from either naïve, PC_{M}-Q11 immunized, or PC_{M}-Q11/CpG immunized mice on ice for 30 min followed by staining with anti-mouse IgG-PE (Clone: Poly4053) (BioLegend, B363203) and anti-mouse IgM-APC (Clone: RMM-1) (BioLegend, B371986) in flow buffer for 30 min on ice. Cells were then washed, and Apotracker Green (BioLegend, 427403) was added for 15 min at room temperature for the detection of apoptotic cells. Cells were then washed in flow buffer and DAPI was added. Flow cytometry was then conducted on a BD FACSCanto II cytometer and the results were analyzed using FlowJo software.

Passive Transfer. Serum from either unimmunized, PC_{M}-Q11 immunized, or PC_{M}-Q11/CpG immunized mice was collected and pooled for each group. 200 μL of serum was then injected via the tail vein into naïve, age-matched mice. Small serum samples were taken the next day, and anti-PC antibody levels were confirmed using ELISA.

Immunofluorescence Imaging and Analysis. Immunofluorescence was conducted on sections from proximal colon samples frozen in OCT and sectioned. Slides were first thawed and outlined with a hydrophobic pen (ImmEdge Hydrophobic Barrier PAP Pen, Vector Labs, H-4000) then fixed with 4% paraformaldehyde for 10 min at room temperature. The slides were washed three times with wash buffer (1×PBS+100 mM CaCl₂+100 mM MgCl) for 10 min at room temperature then incubated in a blocking solution containing 0.3% Triton X-100 and 10% normal donkey serum in 1×PBS+100 mM CaCl₂+100 mM MgCl for 1 h at room temperature. The slides were then incubated in the primary antibodies (rat anti-ZO-1, clone R40.70, Fisher Scientific, MABT11; rabbit anti-occludin, clone EPR20992, Abcam, ab216327) diluted 1:100 in the blocking solution overnight at 4° C. The slides were washed three times with wash buffer for 10 min at room temperature. They were then incubated in the secondary antibodies (donkey anti-rat Alexa Fluor™ 568, Thermo Fisher, A78946; donkey anti-rabbit Alexa Fluor™ 647, Thermo Fisher, A31573) and nuclear marker DAPI diluted 1:500 in the blocking solution for 2 h at room temperature. The slides were washed three times with wash buffer for 10 minutes then allowed to dry at room temperature. The slides were dehydrated in ascending concentrations of ethanol (50-100%), incubated in xylene, and mounted in mounting medium (DPX, Electron Microscopy Sciences, 13512). A Nikon-C2 laser scanning confocal microscope with a 4× or 20× air objective was used to take fluorescent images represented as maximum intensity projections. One 4× and three 20× images were taken of each sample. Imaging and analyses were performed by researchers blinded to treatment group. For the analyses, three 100 μm×100 μm regions of interest within each image were used. Regions of interest were selected to include cross sections of crypts and to avoid crypt edges. The intensities of each marker were quantified using ImageJ and expressed as the integrated density of the fluorescent signal normalized to the area fraction of DAPI positive pixels. All measurements were averaged across the three images per sample and presented per animal.

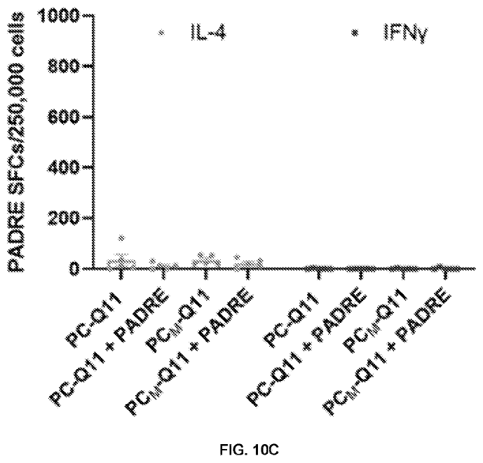

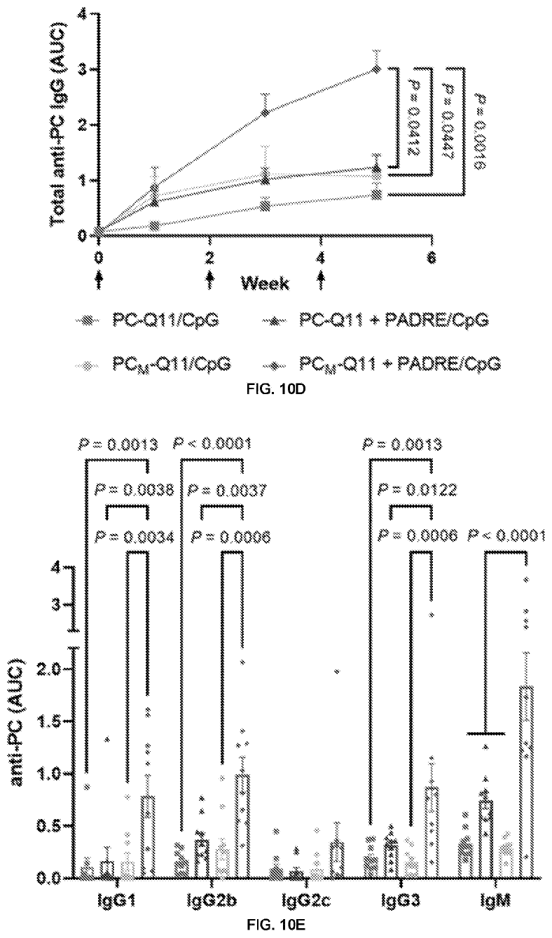

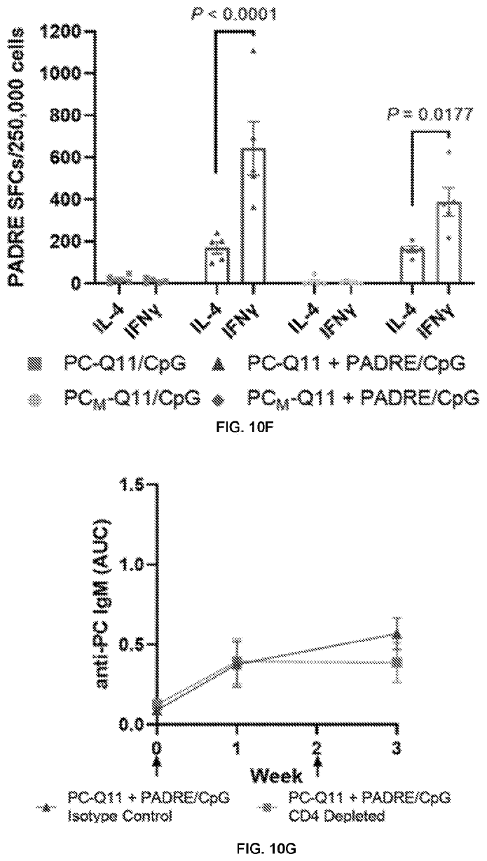

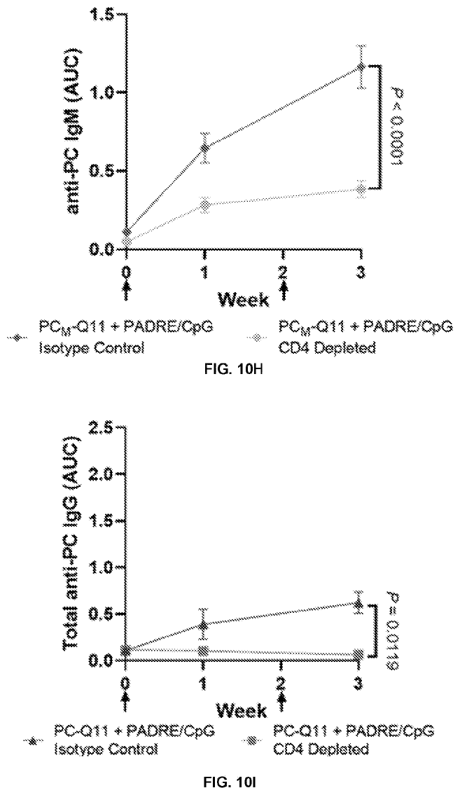

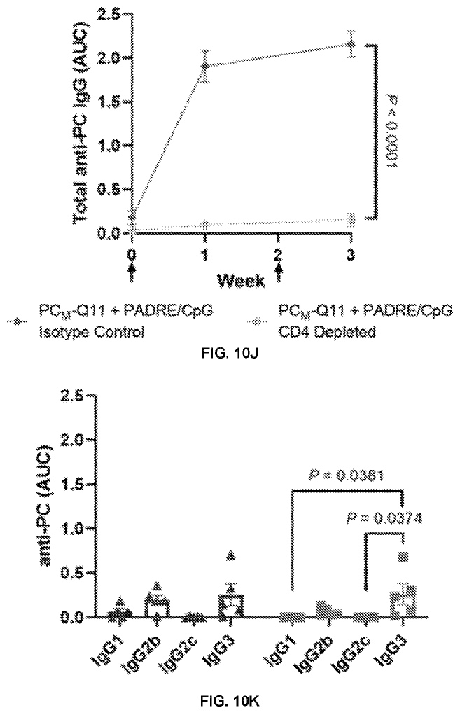

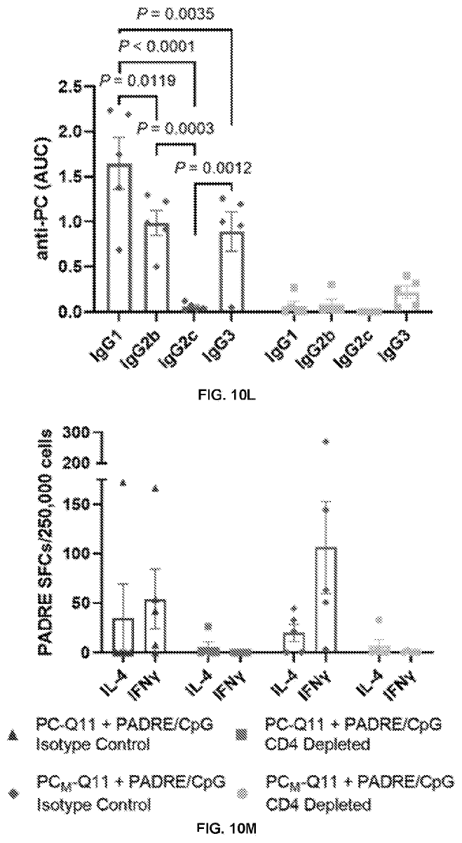

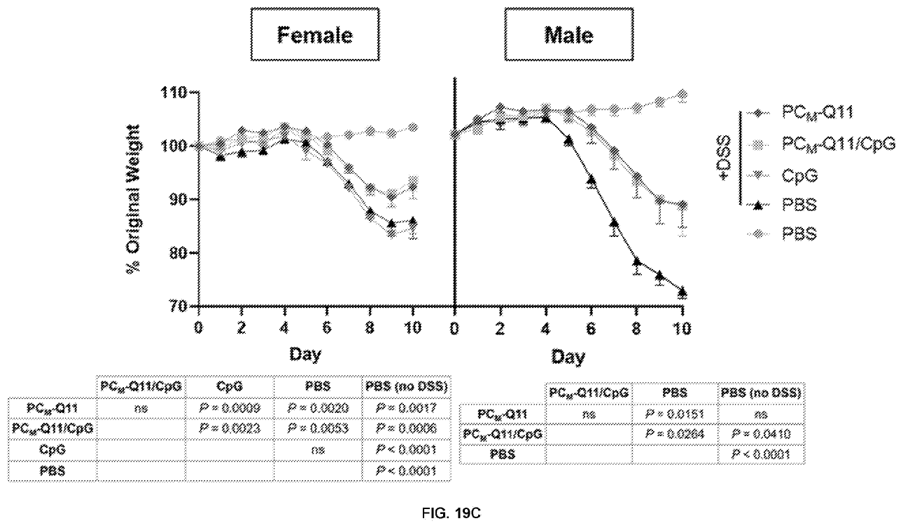

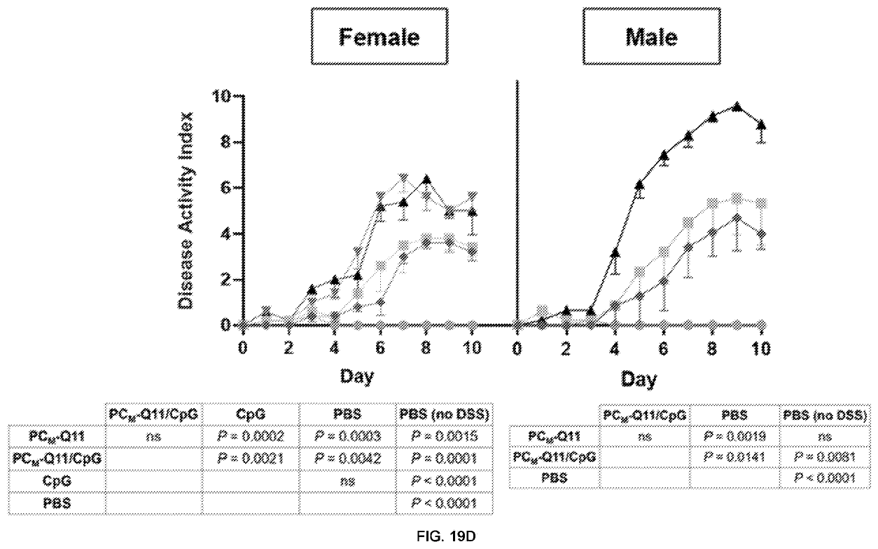

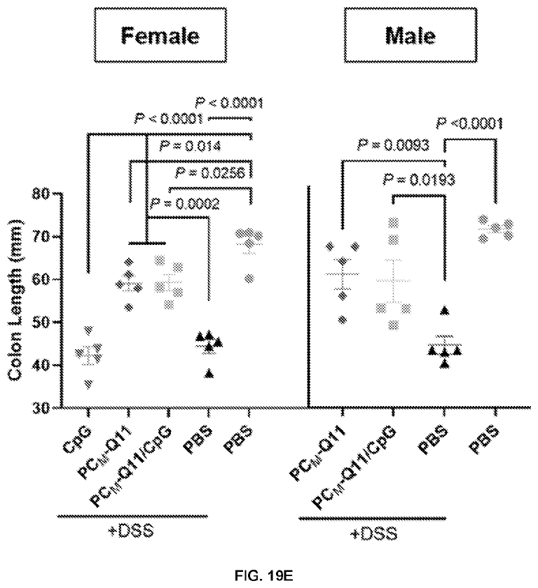

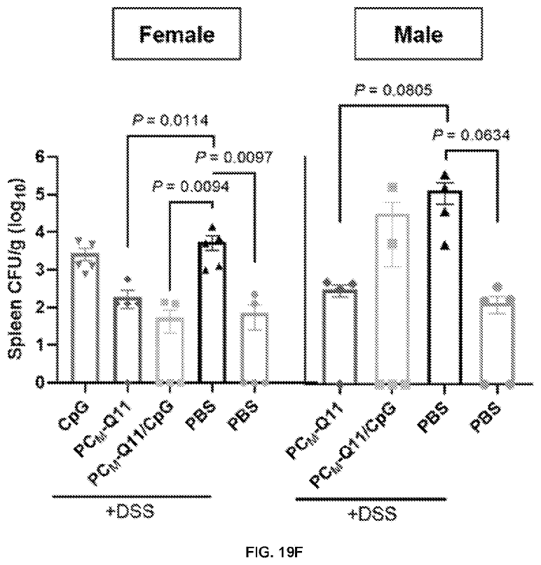

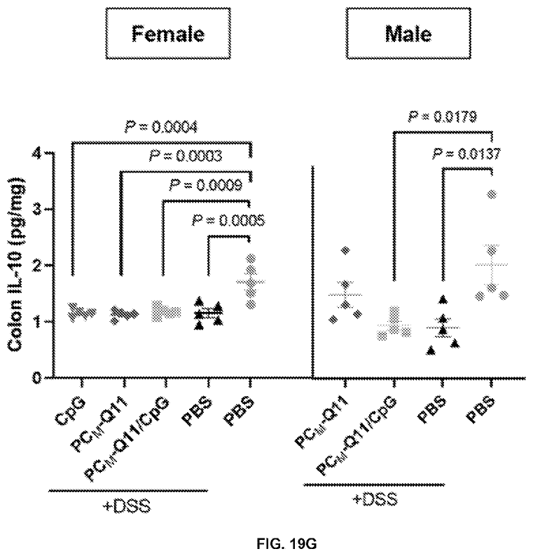

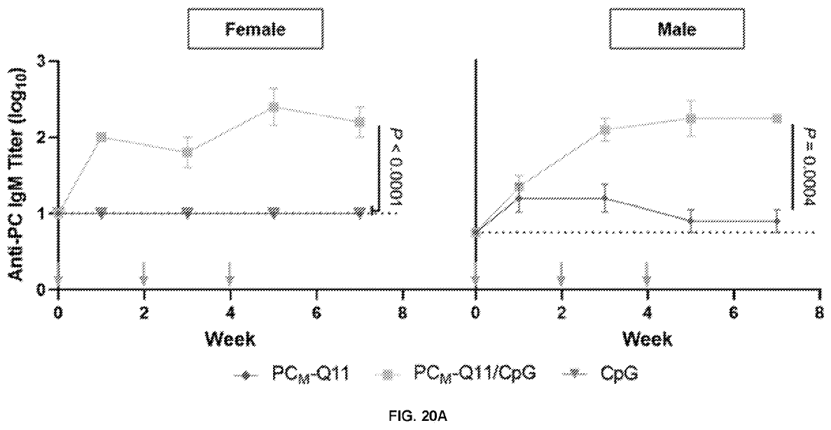

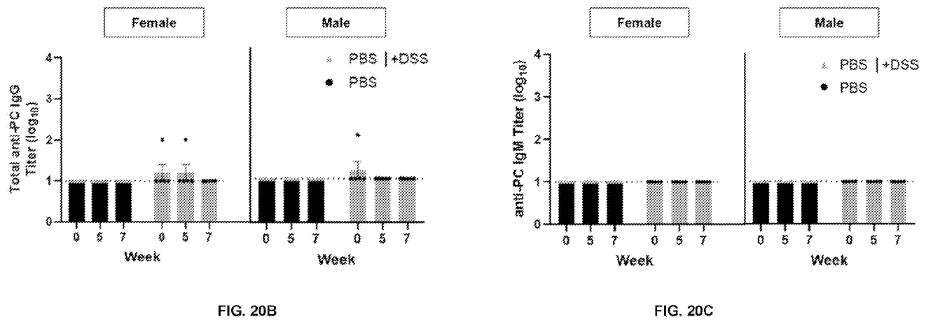

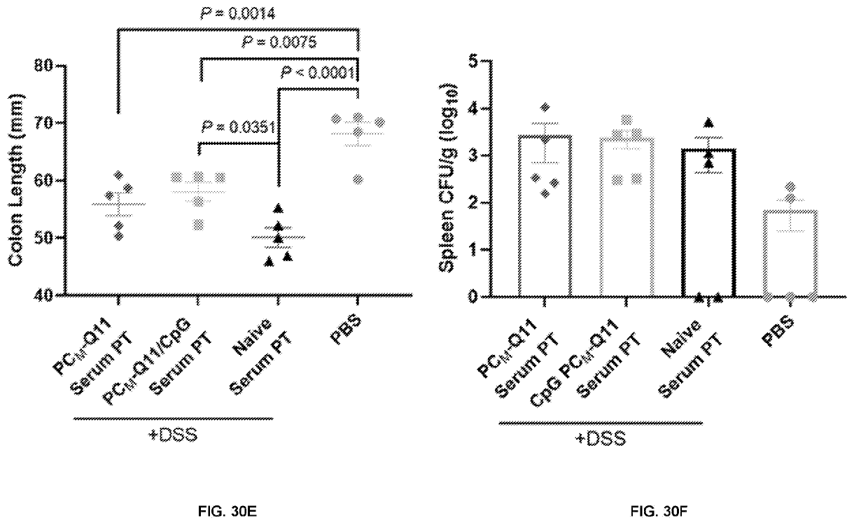

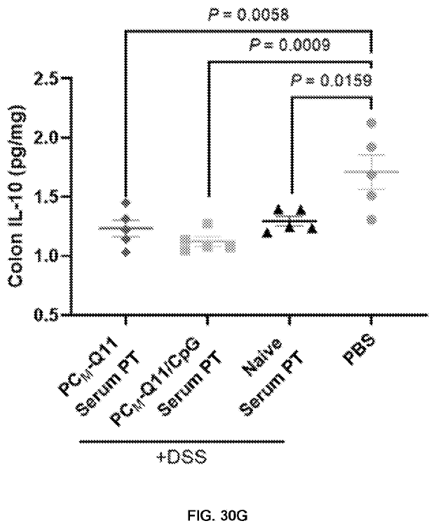

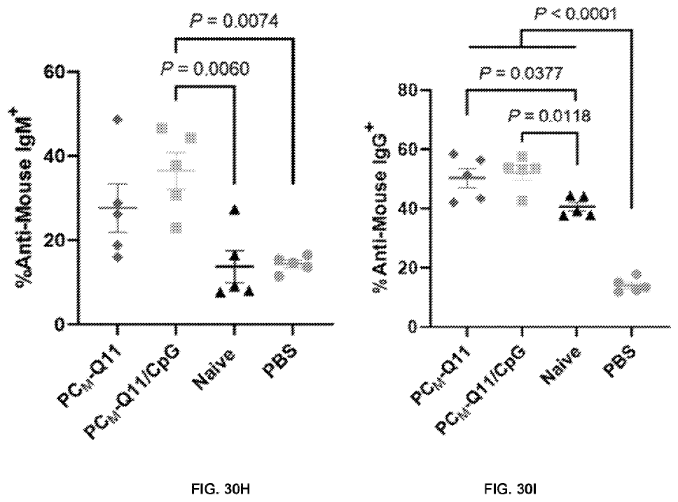

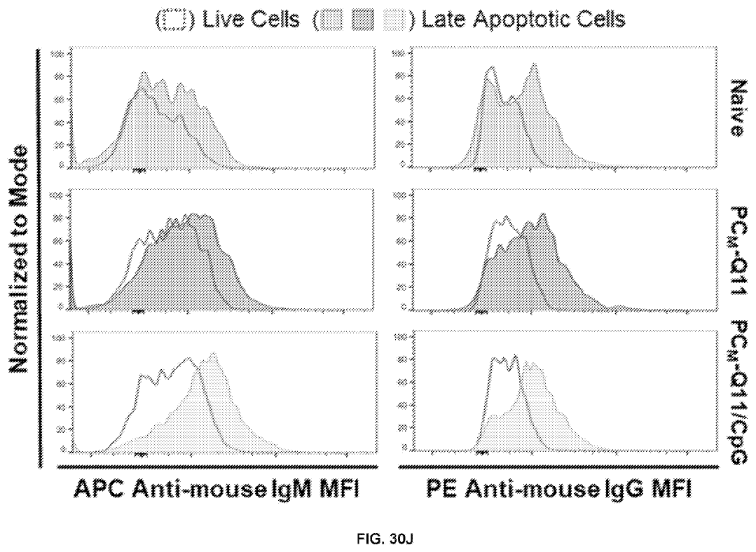

Statistical Analysis. Statistical analyses were performed using GraphPad Prism software. Means+, −, or ±standard error of the mean (SEM) are shown as specified in figure captions along with sample sizes. Each statistical test utilized as well as P values and their significance levels are also indicated in the figures and figure captions. For most data, one-way ANOVA, two-way ANOVA, or two-way RM ANOVA with Tukey's multiple comparison test were used. For ELISpot results, two-way ANOVA with Sidak's multiple comparison test was used. For spleen bacteria measurements, one-way ANOVA with Dunnett's multiple comparison to the disease control was used. Further, a mixed-effects analysis was used for disease activity index data as missing values for some days occasionally occur when mice fail to produce a fecal sample in the time allotted. Mixed-effects analysis were also used for some colitis weight loss curves, as noted in figure captions, when a mouse reached humane endpoints before the experimental endpoint and therefore left missing values for the remaining days of the experiment. This occurred in the male mice DSS colitis experiment where one mouse in the PBS with DSS group and one mouse in the PC_{M}-Q11 immunized group reached humane endpoints on days 8 and 9, respectively. Their spleens could also not be processed and prepared for bacterial measurements with the same protocol and were therefore excluded. For the second chronic colitis experiment, one mouse in the PBS with DSS group reached humane endpoints at day 28. This also excluded this mouse from the spleen bacteria, FITC-dextran, and B cell flow cytometry data. For the Il10^{-/-} colitis experiment, one mouse in the control immunized group was found dead of an unknown cause prior to the induction of colitis. One mouse in the PBS with piroxicam group reached humane endpoints on day 14 and was therefore excluded from B cell flow data. In FIGS. 10A-10M, the Q11 group only contains 9 mice as the tenth was a misinjection with the fluorescent nanofiber solution seen in the subcutaneous space as opposed to the intraperitoneal space as was intended. Further, the PBS without DSS control group shown in FIGS. 19A-19G for the female mice and FIGS. 30A-30J is the same, duplicated in the figures for comparison with the other groups, as these experiments were run in parallel.

### Example 2

### Design and Characterization of Self-Assembling PC-Peptides

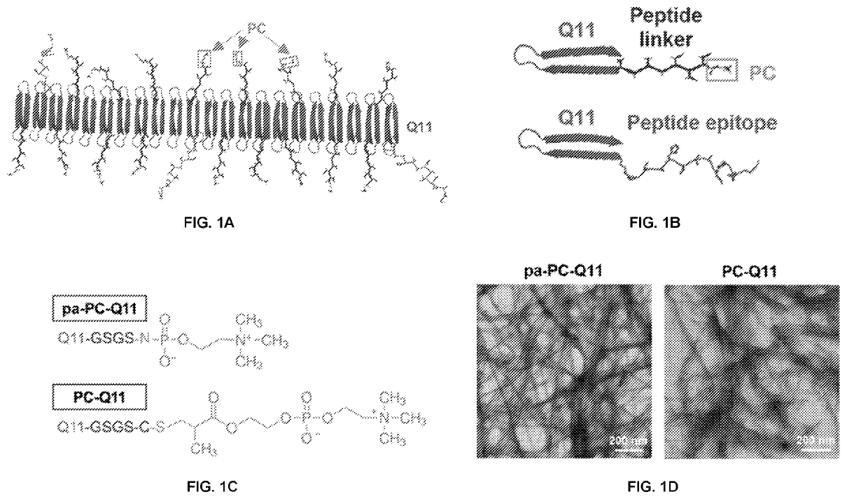

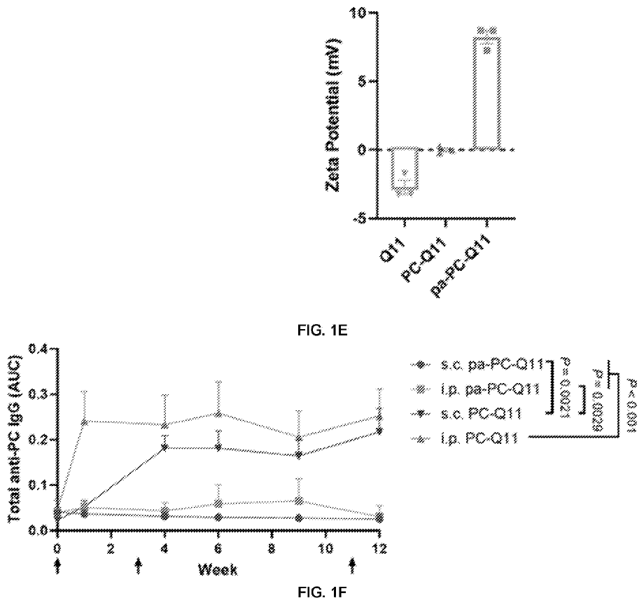

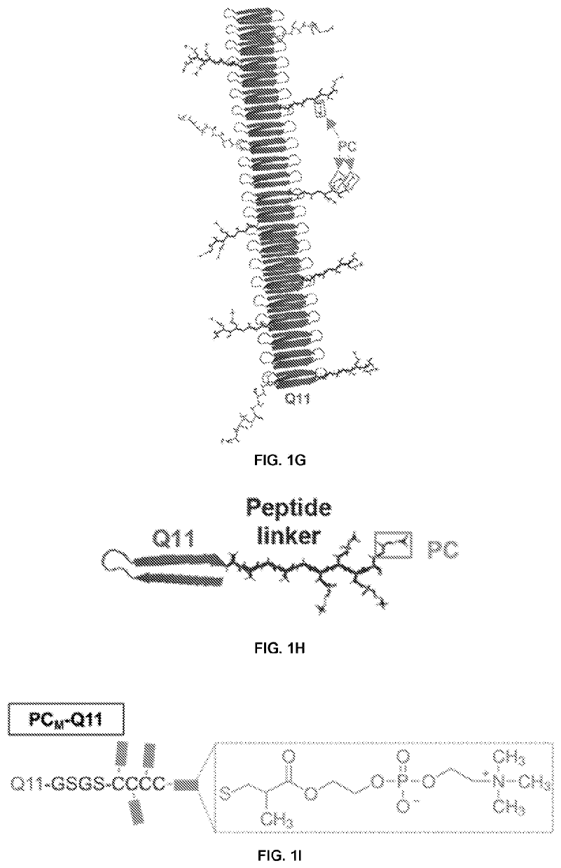

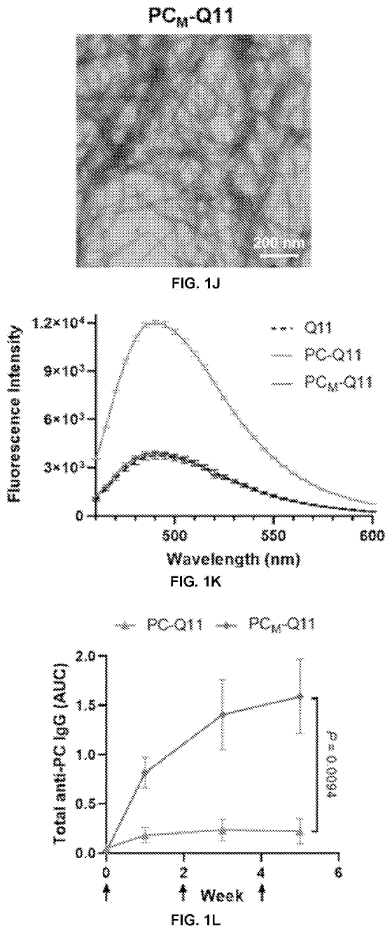

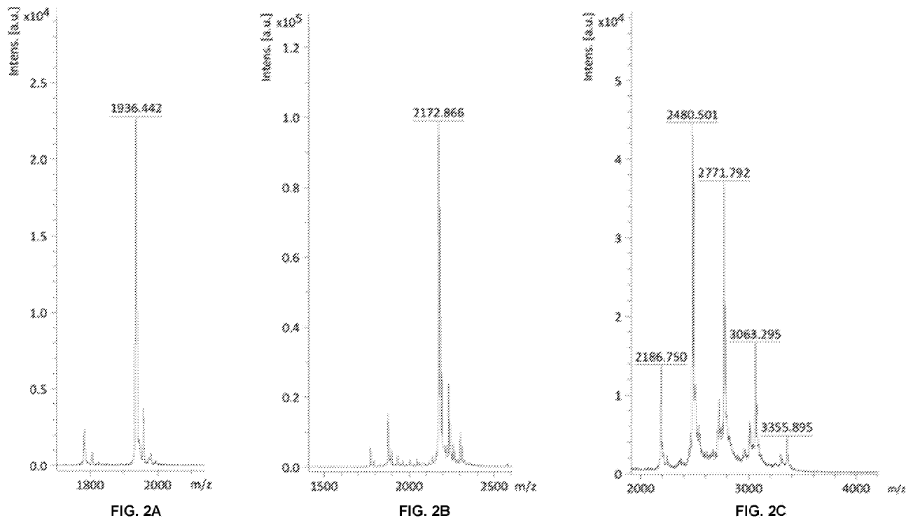

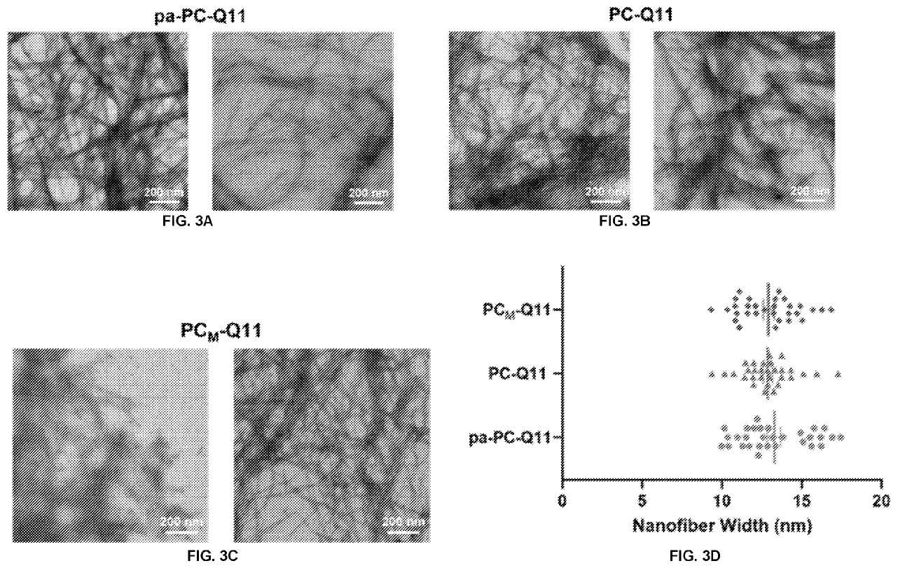

We designed multi-epitope peptide nanofibers bearing titratable amounts of both PC and T-helper epitopes, synthesizing each component separately and co-assembling them to form nanofibers (FIGS. 1A-1B). All components were produced using solid phase synthesis techniques and bore a C-terminal Q11 domain to drive self-assembly into nanofibers, an N-terminal epitope domain (PC or a peptide epitope), and an intervening (Ser-Gly)₂ linker (FIG. 1B, see peptide sequences in TABLE 1). To produce PC-Q11 conjugates, we compared both phosphoramidate and phosphodiester linkages. Phosphoramidate PC-Q11 (pa-PC-Q11, FIG. 1C) was produced by activating PC with EDC/NHS and reacting it with the peptide N-terminus on-resin. Phosphodiester PC-Q11 was synthesized by reacting 2-methacryloyloxyethyl phosphorylcholine with cysteine-terminated Cys-(Gly-Ser)₂-Q11 peptide via Michael addition, leaving the PC phosphodiester bond intact (PC-Q11, FIG. 1C). We initially explored these two conjugation schemes in parallel to select the option providing the best balance between immunogenicity, stability, and ease of synthesis. Both conjugation schemes were confirmed by MALDI-TOF mass spectrometry (FIGS. 2A-2B), and both conjugates formed nanofibers when coassembled with 50% unmodified Q11, with negative-stained TEMs showing nanofibers having morphologies similar to previously investigated Q11-based nanofibers (average widths about 13 nm, lengths of up to hundreds of nanometers) (FIG. 1D, FIG. 3A, FIG. 3B, FIG. 3D). Zeta potential measurements indicated that PC-Q11 nanofibers were neutrally charged, as expected of their zwitterionic structure, whereas pa-PC-Q11 nanofibers were positively charged (FIG. 1E).

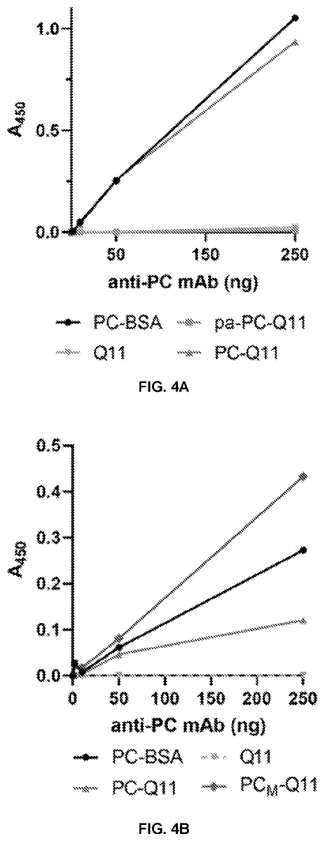

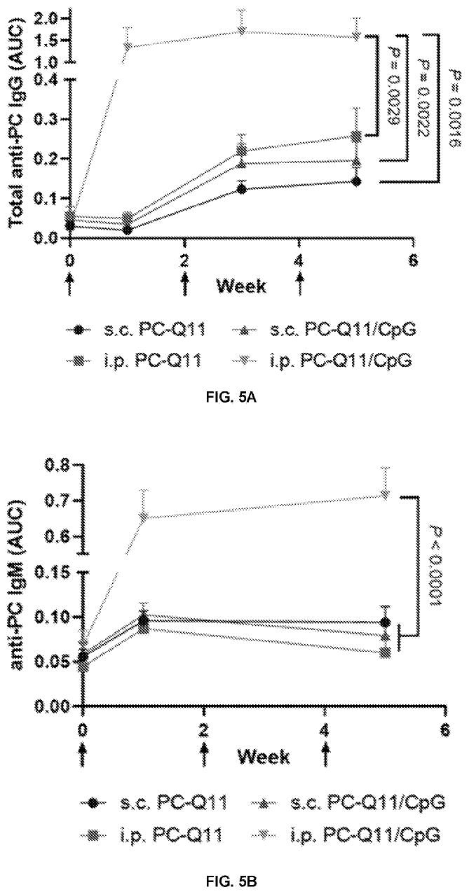

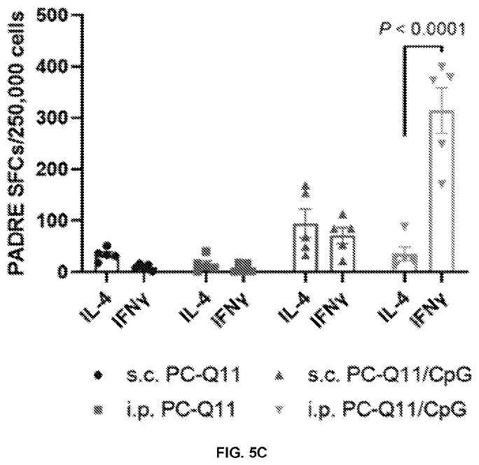

We next probed the reactivity of each conjugate nanofiber with a monoclonal anti-PC IgM antibody, using PC-BSA (bovine serum albumin) as a positive control. PC-BSA is commonly used to detect anti-PC natural antibodies as it presents the PC epitope with a similar structure to how it appears naturally on, for example, apoptotic cells and some bacteria. The antibody bound PC-Q11 but not pa-PC-Q11, indicating that the antigens generated by the phosphodiester linkage are more likely to resemble the epitopes that generate natural anti-PC antibodies (FIG. 4A). Indeed, when mice were immunized either subcutaneously (s.c.) or intraperitoneally (i.p.) with nanofibers containing the T-helper epitope PADRE-Q11 and either PC-Q11 or pa-PC-Q11, PC-Q11 nanofibers elicited a significantly greater antibody response against PC-BSA via both routes, with a recall response seen at week 11 (FIG. 1F). To select the route, boosting schedule, and adjuvant combinations maximizing antibody responses against PC-Q11, we subsequently immunized mice s.c. or i.p. with PC-Q11+PADRE with or without the addition of CpG adjuvant. CpG has been shown to increase IgG3 antibody responses which are integral against bacterial targets, so we expected it to be a suitable adjuvant for enhancing therapeutic anti-PC responses. CpG improved anti-PC IgG and IgM magnitudes as well as T-cell responses, and these were significantly greater for the i.p. route, with these responses stable after three immunizations (FIGS. 5A-5C). In sum, these initial findings highlighted the importance of the phosphodiester bond for PC epitope integrity and indicated that i.p. immunization with CpG adjuvant produced a robust antibody response.

### Example 3

Augmenting Anti-PC Immunogenicity with Multivalency

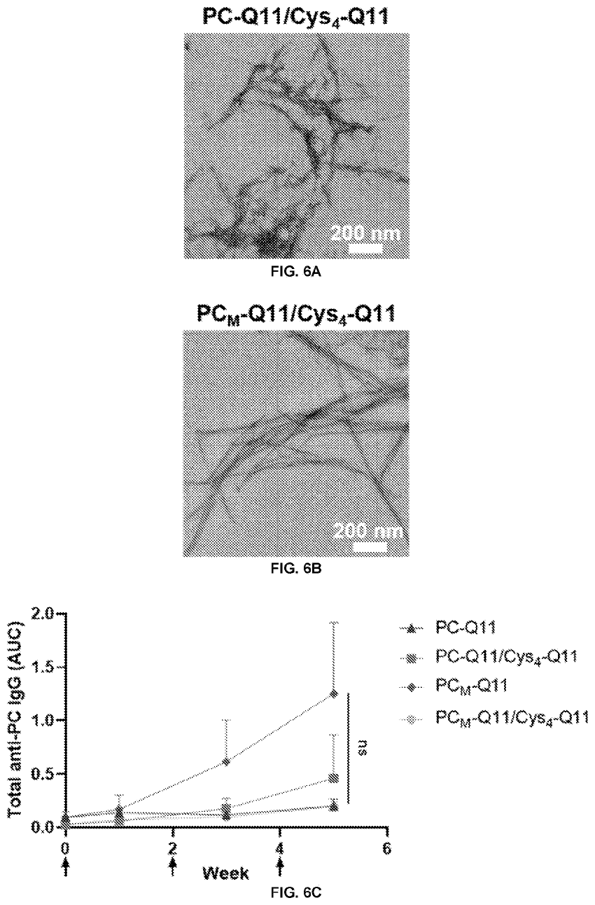

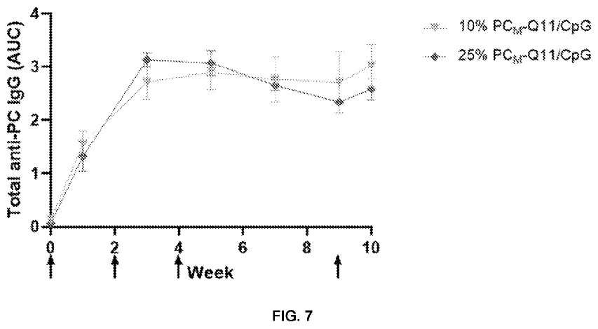

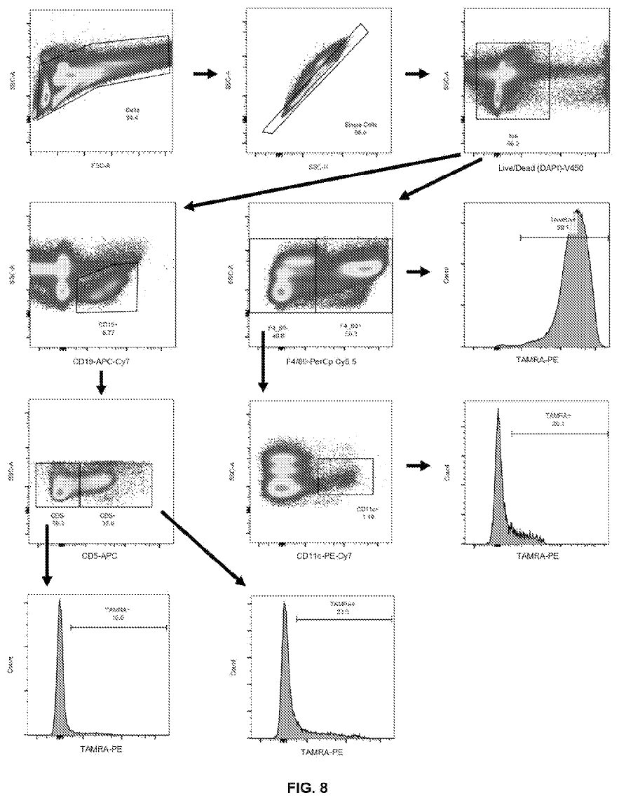

We next sought to augment anti-PC antibody responses further by adjusting the spacing and density of PC on nanofibers. The multivalent display of epitopes has been shown to improve B-cell receptor (BCR) crosslinking and enhance T-independent responses. While different ideal epitope spacing and copy numbers have been investigated, the reported optimum epitope density varies, and material flexibility plays a role (Veneziano, R. et al. Nat. Nanotechnol. 2020, 15, 716-723, incorporated herein by reference). Consequently, we set out to synthesize a PC-peptide conjugate with multiple closely spaced PC molecules per PC-peptide and created a linear multi-PC conjugate termed PC_{M}-Q11, where the “M” stands for “multi” (FIG. 1G). This was achieved by including four cysteine residues on the N-terminus of (Ser-Gly)₂-Q11, providing four thiols for potential PC attachment (FIGS. 1H-1I). One to four PC molecules were conjugated per PC_{M}-Q11 peptide (FIG. 2C). 2 mM PC_{M}-Q11 nanofibers were formed via the co-assembly of 0.2 mM PC_{M}-Q11 and 1.8 mM Q11, representing a similar number of PC molecules per peptide when compared to PC-Q11 while maintaining analogous nanofiber morphology including nanofiber width (FIG. 1J, FIGS. 3C-3D). Both PC-Q11 and PC_{M}-Q11 bound thioflavin-T (ThT), with the presence of a peak fluorescence intensity around 488 nm, regardless of the magnitude of the intensity, indicating that the β-sheet fibrillar structure was preserved for both nanofiber constructs (Groenning, M. et al. J. Struct. Biol. 2007, 158, 358-369, incorporated by reference) (FIG. 1K). Multivalent PC_{M}-Q11 nanofibers also exhibited increased binding of an anti-PC mAb compared to PC-Q11 nanofibers and PC-BSA by ELISA (FIG. 4B). Furthermore, anti-PC IgG responses to i.p. immunization with PC_{M}-Q11 were significantly greater than i.p. immunization with PC-Q11, indicating the superior immunogenicity of PC_{M}-Q11 (FIG. 1L). This enhanced immunogenicity was not attributable to the presence of free thiols, because the addition of extra cysteines (10% Cys₄-Q11) to nanofiber formulations did not significantly enhance or reduce the anti-PC antibody responses generated against PC-Q11 or PC_{M}-Q11, respectively (FIGS. 6A-6C). Additionally, when the epitope content of PC_{M}-Q11 in nanofibers was increased 2.5 times from 0.2 mM (10%) to 0.5 mM (25%) and anti-PC antibody levels elicited from i.p. immunization with both formulations with CpG were compared, there was no significant change in the response elicited indicating that 10% PC_{M}-Q11 was an appropriate formulation to balance immunogenicity and dose sparing (FIG. 7).

### Example 4

### Increased PC Valency Enhances B1a Cell Uptake

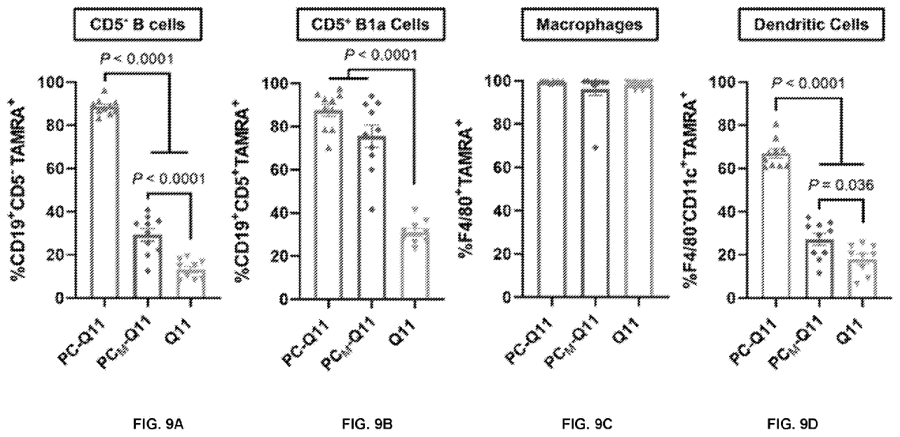

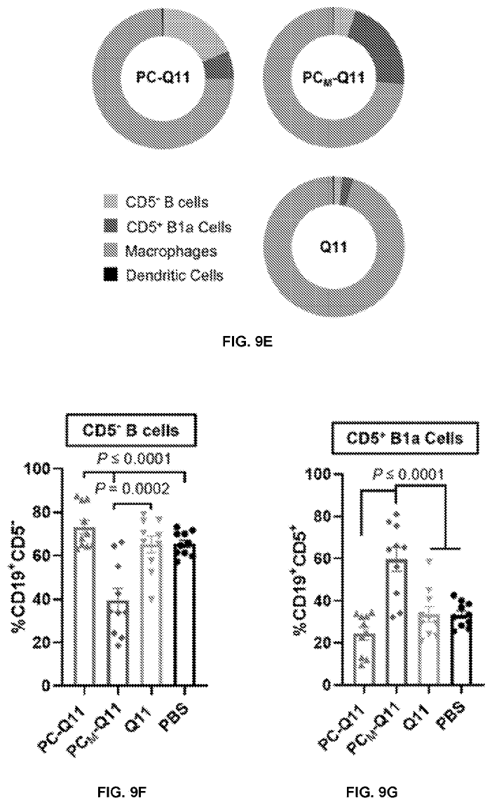

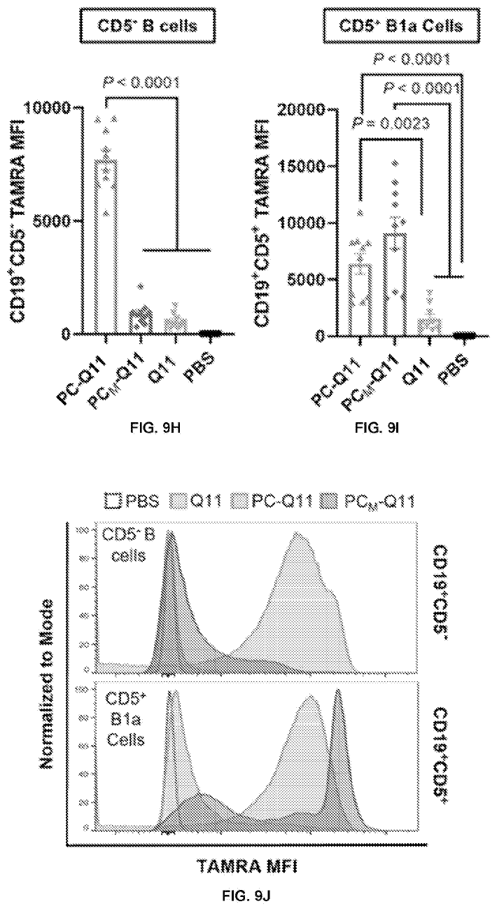

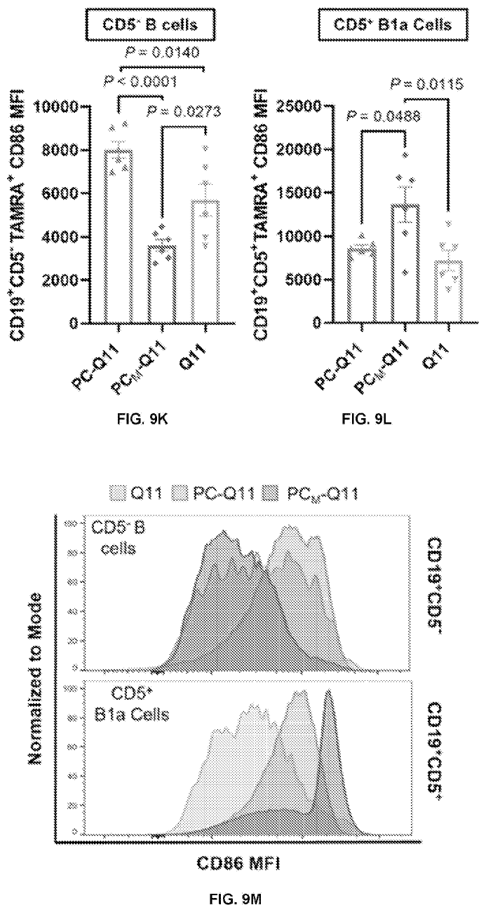

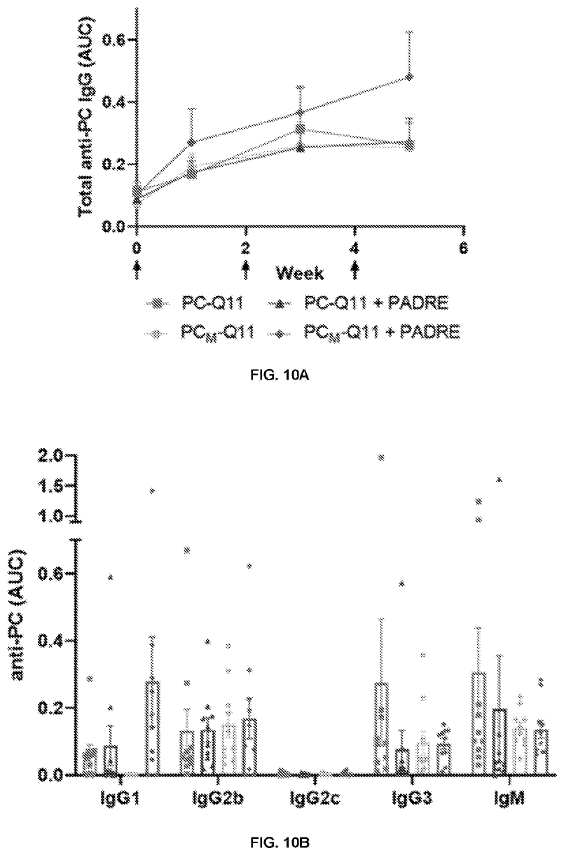

To investigate potential mechanisms behind the elevated antibody responses induced by PC_{M}-Q11 compared with PC-Q11, we studied the interaction of both nanofibers with B1a cells in the peritoneal cavity. Peritoneal B1a cells are known to migrate rapidly upon stimulation and differentiate into NAb-producing plasma cells, with this response beginning by day 1 after stimulation and commonly peaking around day 3. Unlike B1a cells, non-B1a cells are not important for natural antibody production. We thus hypothesized that PC_{M}-Q11 may interact with more B1a cells in the peritoneal cavity, contributing to its ability to produce elevated anti-PC antibody responses. To test this hypothesis, nanofibers were fluorescently labeled with TAMRA and injected into the peritoneal cavity. Four hours later, i.p. lavage fluid was collected, and flow cytometry was used to measure uptake into macrophages (F4/80⁺), B1a cells (CD19⁺CD5⁺), non-B1a B cells (CD19⁺CD5⁻), and dendritic cells (F4/80⁻CD11c⁺) (representative flow cytometry shown in FIG. 8). Peritoneal macrophages acquired Q11, PC_{M}-Q11, and PC-Q11 nanofibers avidly, with almost all macrophages staining positively for nanofibers regardless of nanofiber type. Among B cells and DCs, however, PC_{M}-Q11 and PC-Q11 nanofibers exhibited considerable differences in uptake. PC_{M}-Q11 nanofibers were preferentially acquired by B1a cells, as about 75% of B1a cells stained positively for nanofibers, yet fewer than 30% of DCs or non-B1a B cells acquired PC_{M}-Q11 nanofibers. Conversely, PC-Q11 nanofibers did not show this preference and were acquired by over 80% of non-B1a B cells and about 67% of DCs (FIGS. 9A-9D). This contrast can also be visualized when the total number of TAMRA⁺ cells for each nanofiber is categorized by cell type (FIG. 9E). By this measure, macrophages were the main cell type acquiring nanofibers for all groups, but PC-displaying nanofibers engaged considerable populations of B cells, with PC_{M}-Q11 nanofibers being acquired by a substantial B1a cell population and PC-Q11 nanofibers being acquired primarily by non-B1a B cells. This result indicated PC_{M}-Q11 had enhanced specificity for B1a cells over PC-Q11.

Looking at total cells, regardless of whether they had acquired nanofibers, we additionally found a reduction in non-B1a B cells in the peritoneal cavity in PC_{M}-Q11-injected mice compared with PC-Q11, Q11, or PBS alone (FIG. 9F). Conversely, B1a cells were more prevalent in mice injected with PC_{M}-Q11, with 2 times as many intraperitoneal B1a cells seen compared to PC-Q11-injected mice (FIG. 9G). TAMRA MFI values also illustrated selective uptake of PC_{M}-Q11 into B1a cells and PC-Q11 nanofibers into non-B1a B cells (FIGS. 9H-9J). Furthermore, PC_{M}-Q11 generated enhanced B1a cell activation over non-B1a cells, with increased expression of the CD86 activation marker on TAMRA⁺ cells showing a selective elevation on B1a cells that was not seen for PC-Q11 or Q11 (FIGS. 9K-9M). Together, these results indicated PC_{M}-Q11 had enhanced specificity for B1a cells over PC-Q11, corresponding with previous findings and understanding that increasing the multivalency of B-cell epitopes can improve B cell receptor clustering and consequently antigen internalization and providing a potential explanation for the enhanced anti-PC antibody production seen with PC_{M}-Q11 immunization. Further, these studies focused on the initial uptake of the first immunizations in naïve mice. It may be examined how the uptake of subsequent immunizations of nanofibers may be affected by pre-existing immune responses. This experiment was also conducted using nanofibers lacking T-cell epitopes, so it is not at this time known to what degree, if any, additional T-cell involvement may alter the response observed. Finally, while the substantial uptake by macrophages may be studied further to better understand the role they may play in the responses raised by peptide nanofibers, in the present study we focused on the role of humoral responses and B1a cells motivated by the enhanced specificity for B1a cells and significant antibody responses observed for PC_{M}-Q11 nanofibers.

### Example 5

### Anti-PC Immune Responses are Tailorable

We have previously shown that supramolecular nanofibers can be used to elicit immune responses against endogenous cytokine targets, including TNF and IL-17, via inclusion of the exogenous universal T-cell epitope, PADRE (pan-human leukocyte antigen (HLA) DR-binding epitope), whose use has been verified in C57BL/6 mice. While anti-PC NAbs are mostly generated through T-independent mechanisms, we sought to exploit the modularity of Q11 nanofibers to elicit both T-independent and T-dependent anti-PC responses. This tunability represents a distinct advantage over other technologies, and via the inclusion or exclusion of PADRE-Q11 into PC-Q11 or PC_{M}-Q11 nanofibers, we were able to tailor the antibody and T-cell responses generated and to compare not only the magnitude of these responses but also the biasing towards inflammatory or non-inflammatory phenotypes. PC_{M}-Q11 nanofibers containing PADRE showed a trend toward stronger anti-PC IgG responses compared to those raised with PC-Q11 nanofibers with and without PADRE or PC_{M}-Q11 without PADRE (FIG. 10A). To further characterize those antibody responses, we examined anti-PC IgG subclass levels along with anti-PC IgM. Natural anti-PC antibodies in humans are typically IgM or IgG, with the IgG2 subclass most prevalent. Human IgG2 correlates with mouse IgG3, and both of these are known to be critical in the defense against T-independent bacterial targets. Additionally, IgG2b and IgG2c in mice are typically more inflammatory whereas IgG1 is associated with a more non-inflammatory phenotype. The inclusion of PADRE-Q11 augmented IgG1 responses for PC_{M}-Q11 immunizations, with PC_{M}-Q11+PADRE eliciting a higher IgG1 response than the other groups (FIG. 10B). ELISpot of splenocytes producing IFNγ and IL-4 indicated that i.p. immunization without adjuvant did not generate measurable T-cell responses (FIG. 10C).

To further enhance the immune response to PC-peptide conjugates, we investigated the addition of CpG adjuvant. CpG (ODN 1826) is a commonly used adjuvant, and when administered intraperitoneally, it is also known to improve antibody responses against bacterial targets, with increased IgG3 frequently observed in mice. The magnitudes of antibody responses for all immunizations with CpG were heightened (FIG. 10D). Further, the additional inclusion of PADRE-Q11 to PC_{M}-Q11 showed significant increased anti-PC IgG compared to all other groups when administered with CpG (FIG. 10D). The incorporation of CpG also increased IgM responses, with coassembled PADRE-Q11 responses trending higher and significantly more IgM found in PC_{M}-Q11+PADRE immunized mice compared to all other groups (FIG. 10E). IgG subclass responses were further altered, with the addition of CpG changing the dominant subclasses for PC_{M}-Q11+PADRE, noticeably increasing IgG3 responses as well as showing a marked increase in IgG2b and IgG2c (FIG. 10E). Moreover, splenocyte stimulation with PADRE peptide showed as expected that only PADRE-containing immunizations had significant T-cell responses, and that all mice immunized with CpG were biased towards IFNγ over IL-4 (FIG. 10F). While these antibody and T-cell responses indicated that CpG may bias responses toward a more inflammatory phenotype, the augmented antibody levels including higher IgG3 and IgM suggested that the potential therapeutic benefit of these immunizations warranted further examination. Overall, these results highlighted how multiple formulations may be produced for therapeutic investigation with these modular materials.

Furthermore, to illustrate the importance of PADRE T-cell epitope inclusion in the anti-PC antibody responses elicited by both PC-Q11 and PC_{M}-Q11 immunizations, we examined the anti-PC responses generated in CD4⁺ T-cell depleted mice (FIGS. 11A-11E, representative flow cytometry shown in FIG. 12). Anti-PC IgM was generated by both immunizations in the absence of CD4⁺ T cells but was still significantly greater for PC_{M}-Q11 immunizations when CD4⁺ T cells were present (FIGS. 10G-10H). Conversely, total anti-PC IgG production was reliant on CD4⁺ T cells, with CD4⁺ T cell-depleted mice having significantly smaller anti-PC IgG responses than the isotype control (FIGS. 101-10J). Of the small amounts of anti-PC IgG produced in CD4⁺ T cell-depleted mice, the main subclass elicited was IgG3, which can be produced in a T-independent manner⁴⁰ (FIGS. 10K-10L). PADRE-specific T-cell responses were found in isotype control-treated but not anti-CD4 antibody-treated mice (FIG. 10M). Together, this data suggested that both T-independent natural anti-PC antibody production and T-dependent anti-PC antibody production contribute to the overall anti-PC responses elicited by PC-Q11 or PC_{M}-Q11 immunizations.

### Example 6

### Pc_{M}-Q11 Immunizations are Protective in a Model of Chronic Colitis

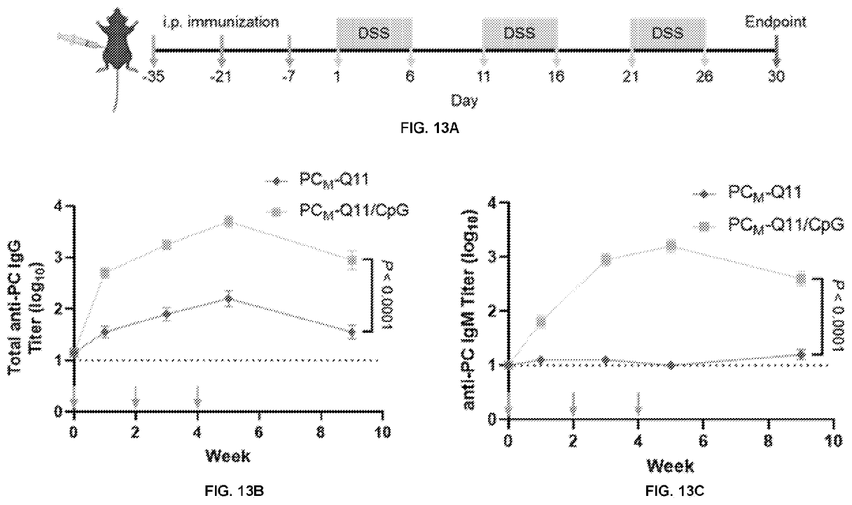

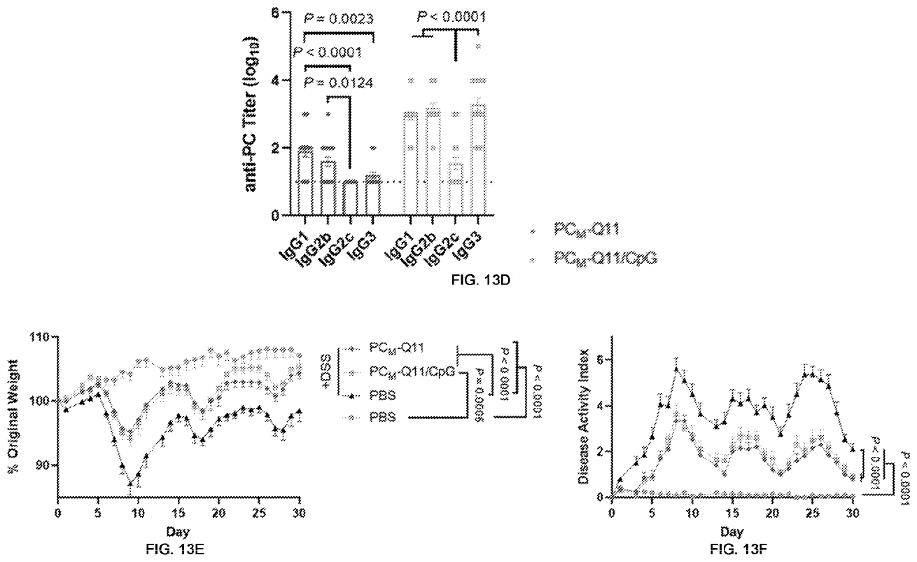

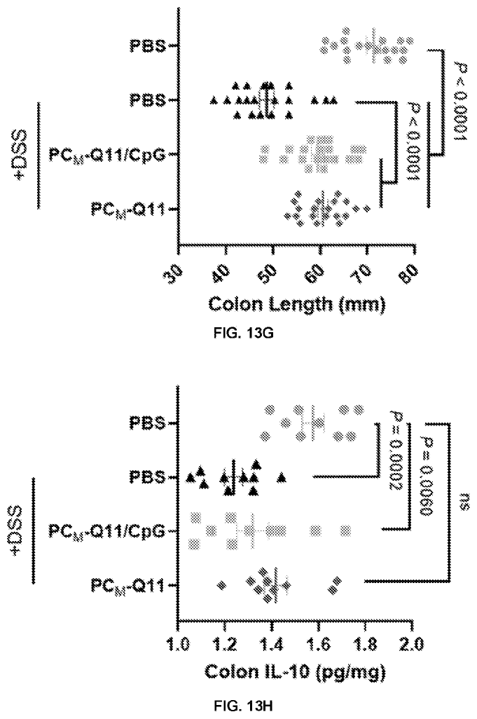

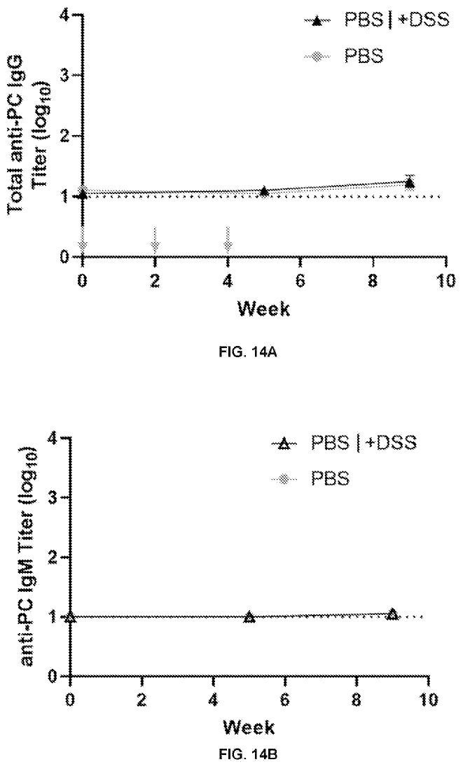

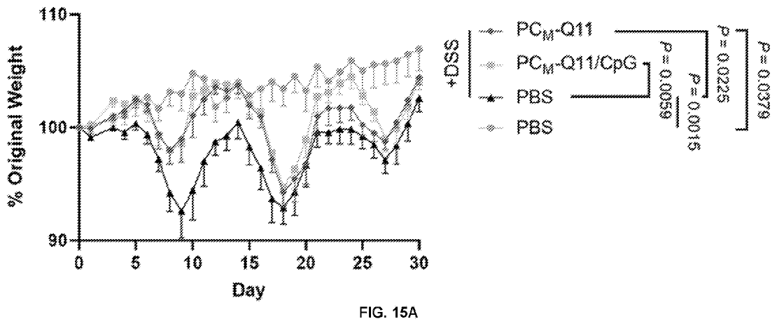

We next investigated the extent to which anti-PC responses raised by peptide nanofibers were therapeutic in a murine model of colitis induced by dextran sodium sulfate (DSS). Previous experiments had indicated that the inclusion of PADRE or CpG in the immunization modulated the strength and phenotype of the B- and T-cell responses raised, but it was not yet clear which formulation may be most therapeutic. On one hand, CpG augmented antibody responses, particularly IgG3 and IgM, which are thought to enhance protection against pathogenic bacteria. On the other hand, CpG also induced a more inflammatory (IFNg-producing) T-cell response, so experimentation in the context of a relevant disease model was necessary to ascertain how these potentially countervailing factors influenced efficacy of the immunizations. In DSS-induced colitis, DSS administered ad libitum in drinking water leads to epithelial cell disruption in the colon, defects in the mucosal barrier, and consequently high levels of inflammation that mimics the disease-state of IBD. In short duration DSS-colitis experiments, innate immune cells dominate the observed pathology, but as the model progresses, defects in lymphocyte function may also play a role, resembling the pathogenesis of chronic IBD. Before the induction of colitis, mice were immunized with either PC_{M}-Q11 or PC_{M}-Q11/CpG, both coassembled with PADRE-Q11, or injected with PBS (for PBS with DSS (disease) and PBS without DSS (healthy) controls) followed by the induction of a 30-day chronic DSS colitis model (FIG. 13A). Mice were immunized three times at weeks 0, 2, and 4, as this was found to produce durable anti-PC responses (FIG. 7). We found that PC_{M}-Q11/CpG induced both greater IgG and IgM responses (FIGS. 13B-13C). PC_{M}-Q11 did not induce anti-PC IgM within detection levels (FIG. 13C). Notably, antibody responses for both immunized groups were slightly reduced at the end of the chronic DSS period (FIGS. 13B-14C). No preexisting natural anti-PC IgG or IgM antibodies were within detection levels for PBS-immunized disease or healthy control mice (FIGS. 14A-14B). A direct comparison between mice who received unadjuvanted or adjuvanted immunizations with CpG indicated that PC_{M}-Q11 skewed the subclass response towards non-inflammatory IgG1 with IgG2b also present whereas PC_{M}-Q11/CpG raised more IgG2b, IgG2c, and IgG3 in addition to IgG1 (FIG. 13D).

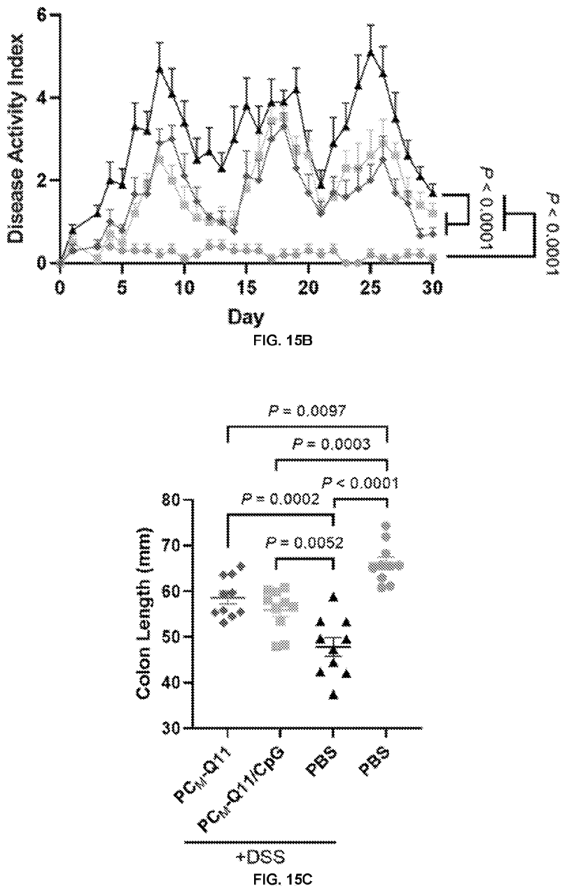

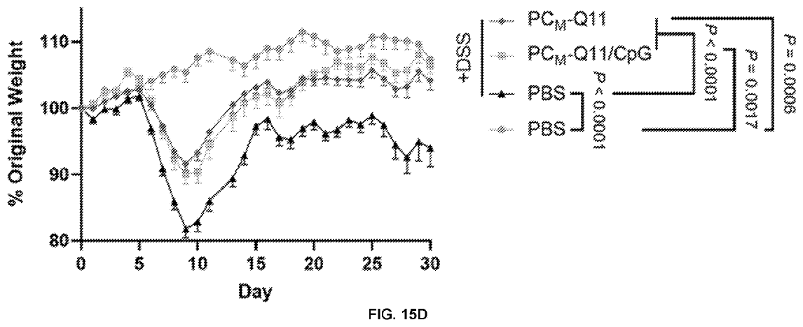

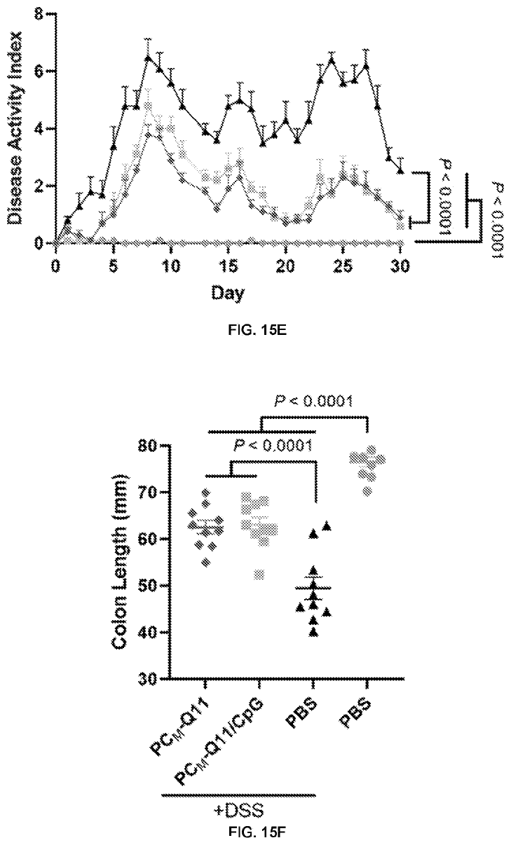

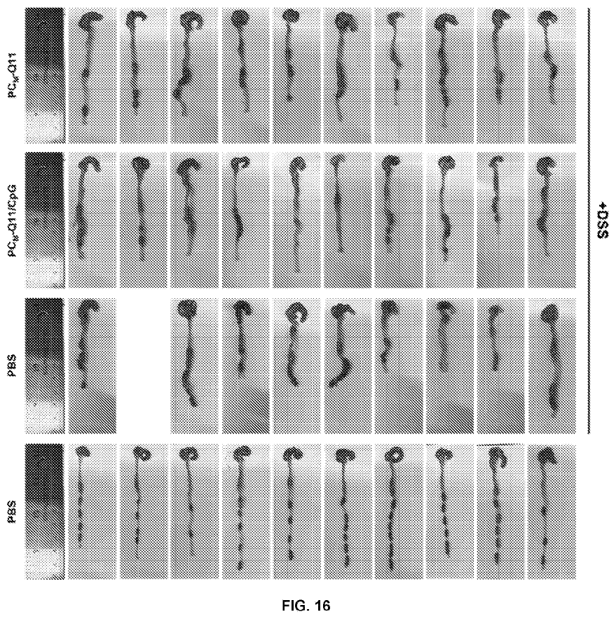

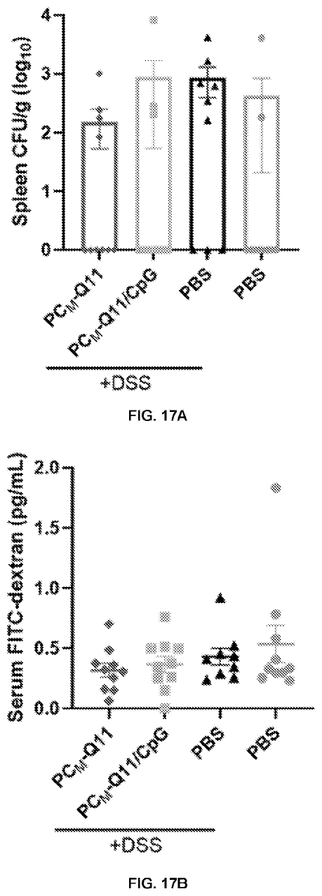

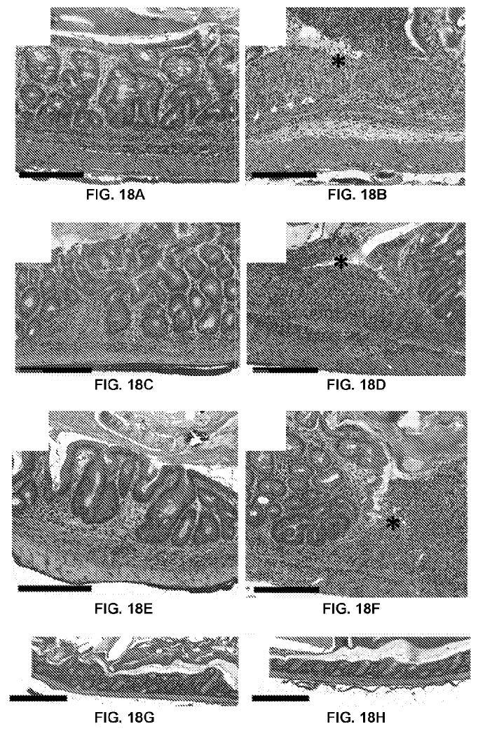

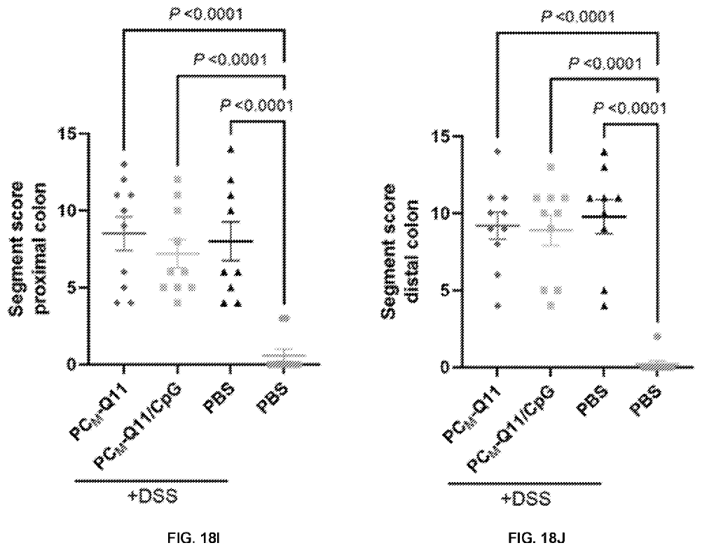

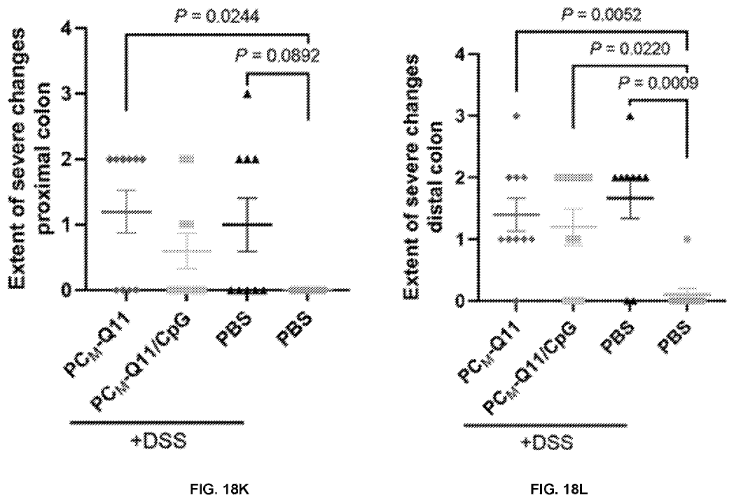

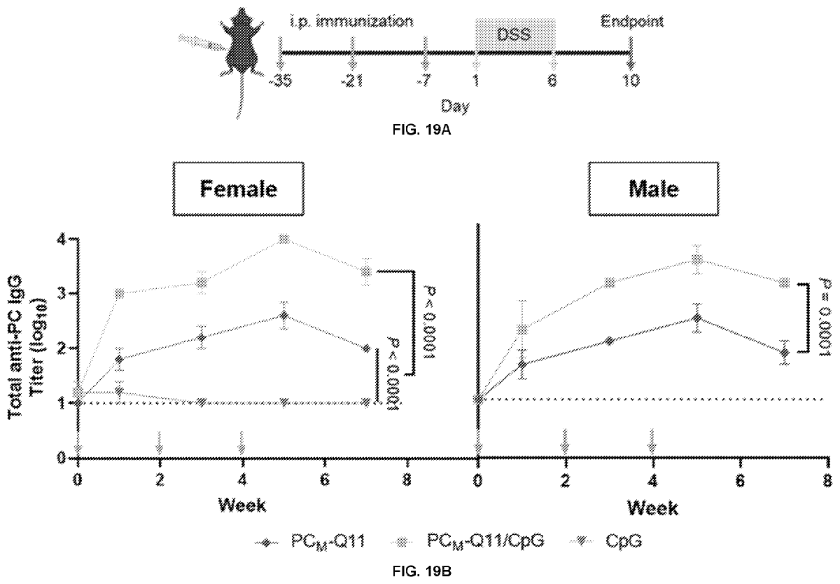

In the chronic DSS model, both PC_{M}-Q11 and PC_{M}-Q11/CpG immunizations exhibited significant protective efficacy in two separate experiments (FIGS. 15A-15F), with the combined results showing reduced weight loss (FIG. 13E), improved disease severity (FIG. 13F), and diminished colon shortening (FIG. 13G). Disease activity index (DAI) scores (FIG. 13F) represented an additive measure of weight loss, fecal consistency, and occult blood, with higher scores indicating more severe disease. Immunized mice had significantly improved DAI scores compared to disease controls, with fecal consistency noticeably more solid and fewer mice presenting with occult blood throughout the chronic model. Both weight loss and DAI scores were cyclic, coinciding with the administration of DSS (FIGS. 13E-13F). After the 30-day evaluation period, mice were euthanized, and their colons were measured, with shorter colons indicating more severe inflammation and damage that resulted in fibrotic shortening. The colons of immunized mice were significantly longer than those of disease controls, providing a clear physical representation of diminished disease severity (FIG. 13G, FIG. 16). Moreover, levels of IL-10, an anti-inflammatory cytokine associated with B1a cells and NAb production, trended higher in colon homogenates from immunized mice, with PC_{M}-Q11-immunized mice having the highest levels that were not significantly different from healthy controls (FIG. 13H). Measures of colon epithelial integrity including bacterial spread to the spleen and leakage into the serum of FITC-dextran given via oral gavage were not significantly different from healthy controls for any DSS-administered group (FIGS. 17A-17B). Histological evaluation of hematoxylin and eosin-stained segments from the proximal and distal colon by a pathologist blinded to treatment group, however, did not show significant histologic improvements for immunized mice when examined 5 days after the end of DSS exposure (FIGS. 18A-18L), although a trend toward reduced extent of severe damage in both the proximal and distal colon segments was evident for the PC_{M}-Q11/CpG immunized group at this early time point (FIGS. 18K-18L). Overall, these assessments illustrated that immunization with PC_{M}-Q11 or PC_{M}-Q11/CpG provided considerable protective benefit in reducing disease severity in a mouse model of chronic DSS-induced colitis.

### Example 7

### Pc_{M}-Q11 Efficacy is Robust and Reduces Viable Bacteria Spread to the Spleen

Given the efficacy of PC nanofiber active immunotherapies that we observed in a chronic model of colitis, we further examined their efficacy in acute colitis induced by one cycle of DSS in both female and male mice, including whether any effects could be attributed to CpG alone. CpG is a TLR-9 agonist that has been reported to improve intestinal barrier function and, when injected intraperitoneally, to have therapeutic effects in models of colitis. These results, however, vary greatly depending on the amount and schedule of CpG administration. Therefore, we investigated whether administration of CpG comparable to the CpG included with PC_{M}-Q11 immunizations contributed to the observed efficacy. Mice were immunized as before, however, only one cycle of DSS-induced colitis was conducted, resulting in a shorter 10-day model (FIG. 19A). We found anti-PC IgG and IgM levels to be similar to previous results for immunized (FIG. 19B and FIG. 20A) and unimmunized groups (FIGS. 20B-20C) in both male and female mice. Additionally, we did not measure high levels of anti-PC IgG or IgM after CpG injection alone (FIG. 19B and FIG. 20A).

Again, we found that PC_{M}-Q11 and PC_{M}-Q11/CpG immunizations exhibited significant protective efficacy in the shorter one cycle DSS acute colitis model in female mice, including, of note, reduced viable bacteria spread to the spleen after colon damage, while CpG alone was not attributed to any improved disease metrics (FIGS. 19A-19G). DSS-induced colitis has been reported to affect male mice more severely. However, both PC_{M}-Q11 and PC_{M}-Q11/CpG immunizations still exhibited protective efficacy in male mice, with improved weight loss and disease activity index scores, similar to female mice (FIGS. 19C-19D). Weight loss and DAI scores in female mice immunized with CpG alone were just as severe as PBS-immunized DSS controls (FIGS. 19C-19D). Further, PC_{M}-Q11 and PC_{M}-Q11/CpG immunized groups demonstrated increased colon lengths compared to CpG-immunized mice and DSS disease controls for both sexes (FIG. 19E). Using bacteria cultured from the spleen as an indication of leakage from the damaged colon (Chassaing, B., et al. Curr. Protoc. Immunol. 2009, 104, 152511-152514, incorporated by reference), it was found that PC_{M}-Q11 and PC_{M}-Q11/CpG immunization dramatically decreased spleen colony forming units (CFUs) in female mice to levels similar to those in mice that did not receive DSS (FIG. 19F). In contrast, CpG-treated and PBS-immunized groups exhibited considerable bacterial spread to the spleen from the damaged colon (FIG. 19F). This trend was also present, though not statistically significant, for male mice immunized with PC_{M}-Q11, whereas male mice immunized with PC_{M}-Q11/CpG had a more bimodal result with two mice having high bacterial spread and three mice having no bacterial spread to the spleen at all (FIG. 19F). Finally, no differences in IL-10 levels were observed for any female mice receiving DSS, but all were lower than healthy controls in female mice, potentially owing to the early endpoint of the experiment compared to more chronic models (FIG. 19G). Colon IL-10 levels in PC_{M}-Q11 immunized male mice, however, were not significantly reduced compared to healthy controls (FIG. 19G). Together, these data conclusively indicated that CpG administered in this manner to female mice was not therapeutic in this model of DSS-induced colitis. Immunization efficacy was, however, reproducible in this shorter model for PC_{M}-Q11 and PC_{M}-Q11/CpG immunized mice and seen across sexes, indicating the robustness of anti-PC active immunotherapy for different DSS regimens.

### Example 8

Therapeutic Immunization with PC_{M}-Q11 Reduces Colitis Disease Severity

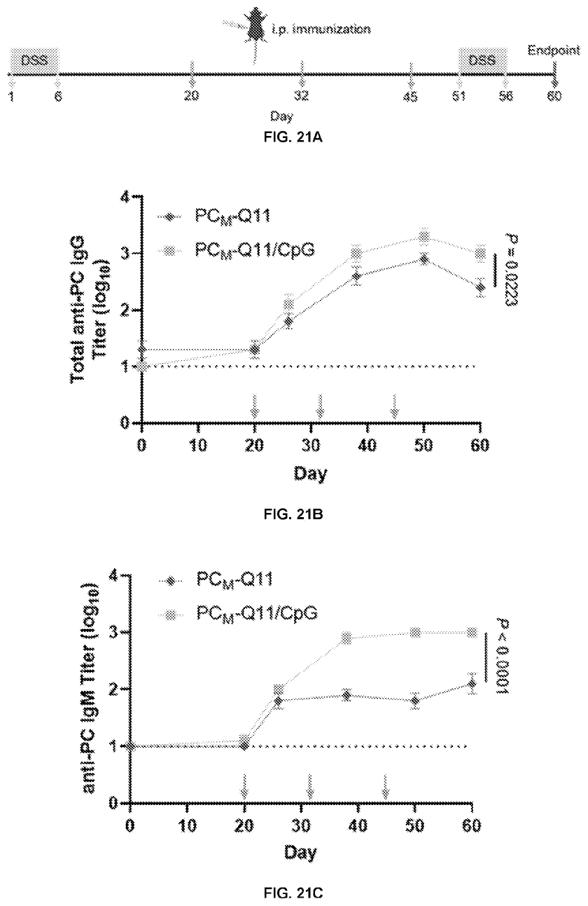

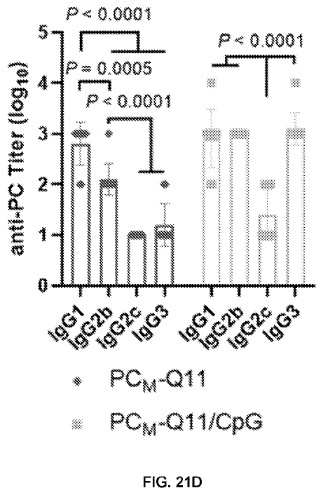

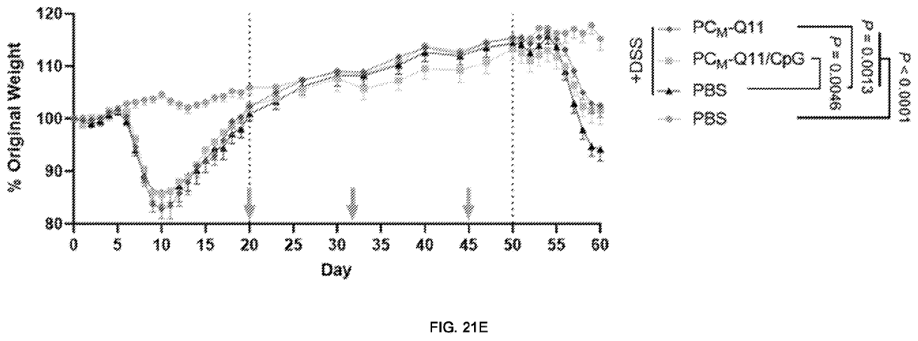

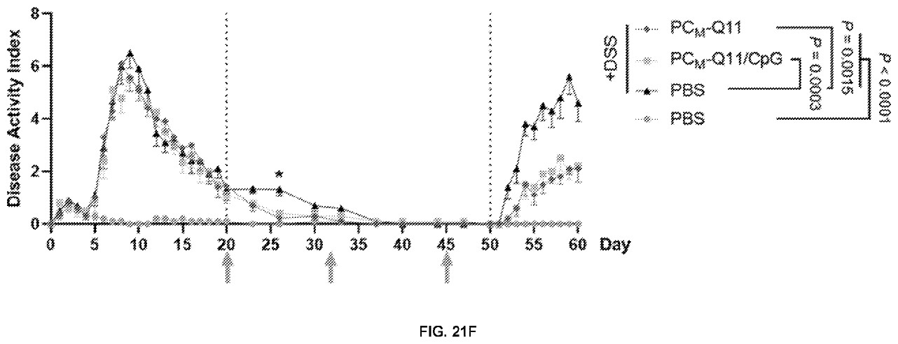

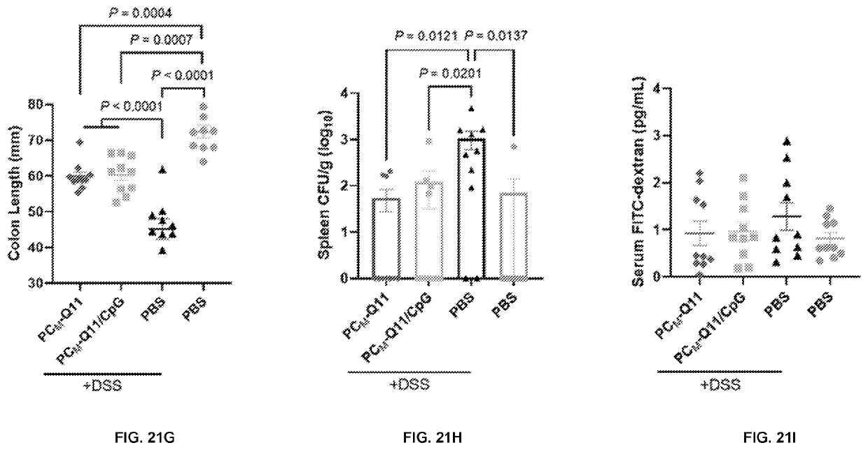

We next sought to determine the efficacy of these immunizations in a therapeutic DSS colitis model rather than a protection model. Here, colitis was induced in naïve mice with five days of DSS administration followed by daily monitoring until day 20, when mice had recovered almost all of their original body weight. Then, mice were immunized three times over four weeks with either PC_{M}-Q11 or PC_{M}-Q11/CpG, both coassembled with PADRE-Q11, then again administered DSS for five days (FIG. 21A). Interestingly, mice with prior DSS colitis raised higher anti-PC IgG responses when immunized with PC_{M}-Q11 without CpG than is typical of this immunization when it is administered in the absence of prior colitis, approaching levels nearer to those elicited by PC_{M}-Q11/CpG immunizations (FIG. 21B). There also was the addition of a measurable anti-PC IgM response, but IgG subclasses were not altered (FIGS. 21C-21D). Only minimal anti-PC IgG or IgM responses were measured in PBS-immunized control mice both before and after DSS colitis (FIGS. 22A-22B).

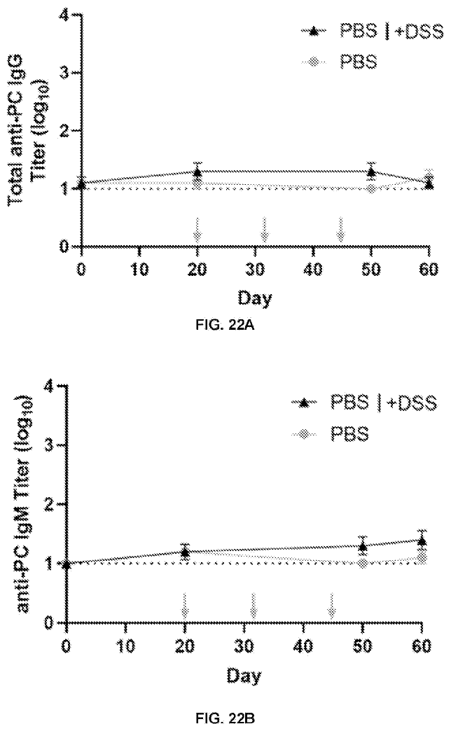

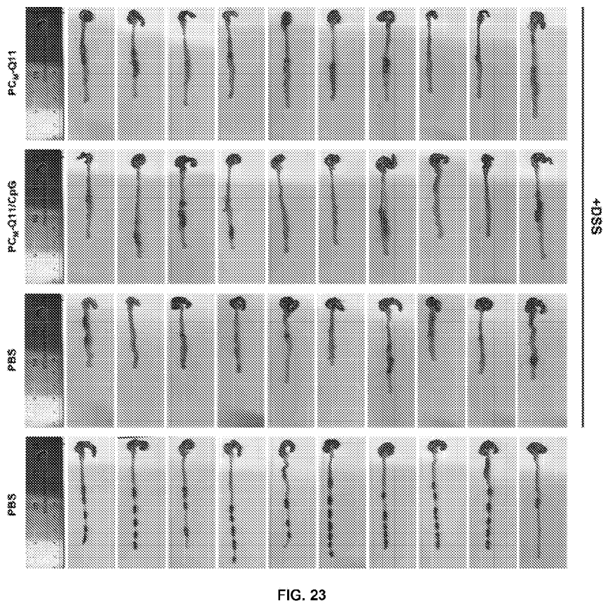

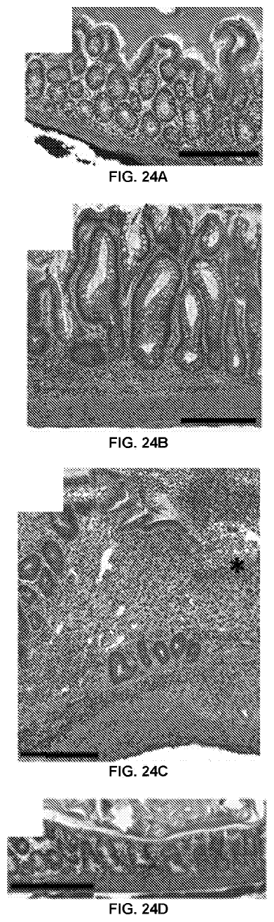

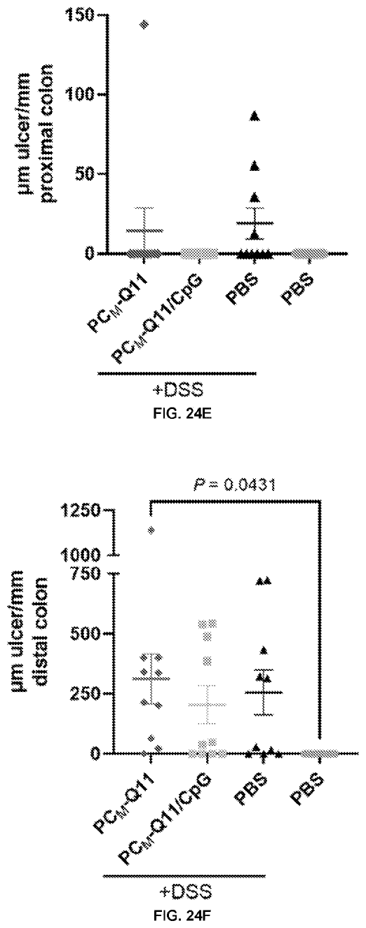

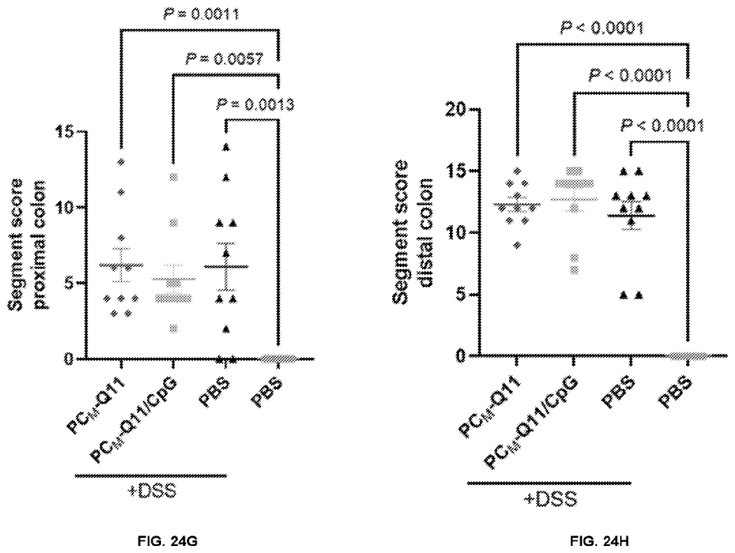

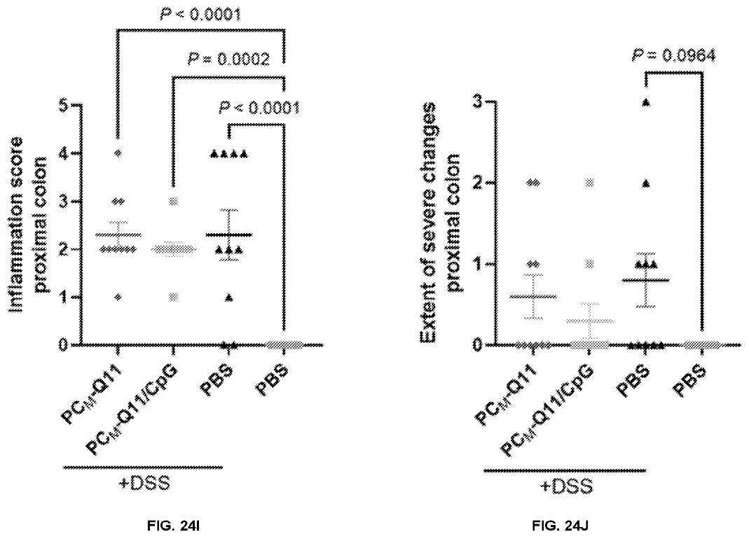

We found that PC_{M}-Q11 immunizations were therapeutic upon the second 10-day cycle of colitis, with both weight loss and disease activity index scores significantly improved (FIGS. 21E-21F). In fact, even after the first immunization, DAI scores began to decrease compared to unimmunized controls (FIG. 21F). Colon lengths were also significantly longer for immunized groups (FIG. 21G and FIG. 23). Further, bacterial spread to the spleen was significantly reduced and comparable to healthy controls for immunized groups (FIG. 21H). However, detection of 3-5 kDa m.w. FITC-dextran in the serum after oral gavage was similar in immunized groups and disease controls (FIG. 21I), suggesting the effect of immunization is not limited to effects on colon mucosal integrity alone. Proximal and distal colon segments taken at the experimental endpoint were evaluated by a pathologist blinded to experimental group (FIGS. 24A-24F). Proximal segments demonstrated fewer ulcers present for PC_{M}-Q11 groups (with or without CpG) compared to disease controls (FIGS. 24A-24E). No differences in ulceration were observed in the distal colon segments (FIG. 24F). However, the total segment scores did not differ between experimental groups for either the proximal or distal colon segments of the DSS-exposed groups (FIGS. 24G-24H). Slight but nonsignificant trends were observed for decreased inflammation and extent of severe changes scores in the proximal colon for the PC_{M}-Q11/CpG immunized group (FIGS. 241-24J). Overall, these results indicated that immunization with PC_{M}-Q11 formulations also reduced clinical disease severity in a therapeutic setting, with decreased weight loss, disease activity scores, and bacterial spread to the spleen, but via mechanisms that did not significantly affect histological scoring performed 5 days after DSS discontinuation.

### Example 9

### Pc Immunization in an Il10^{-/-} Model of Colitis

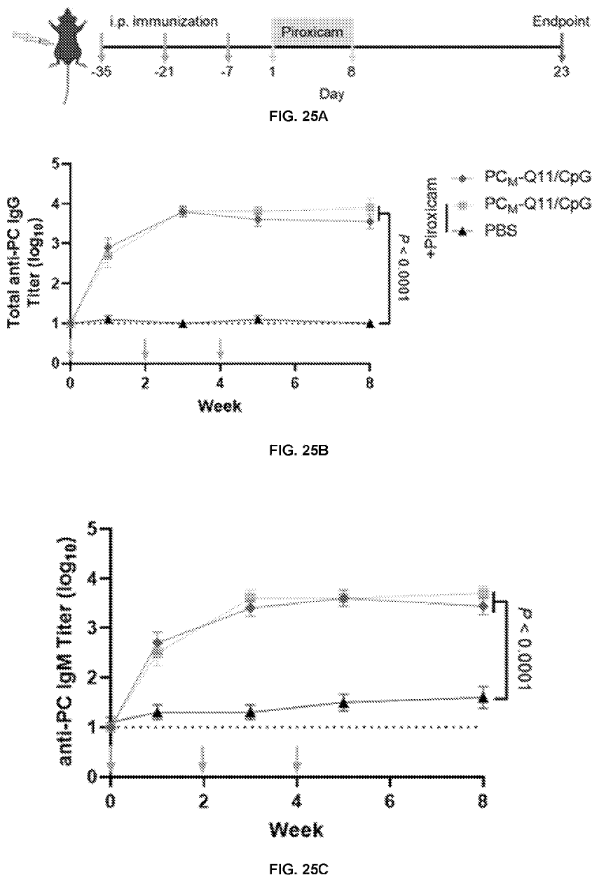

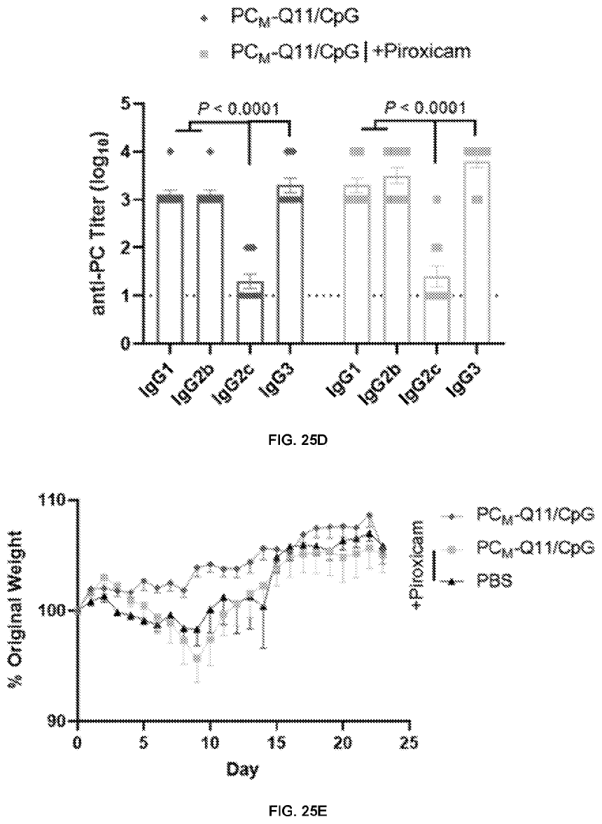

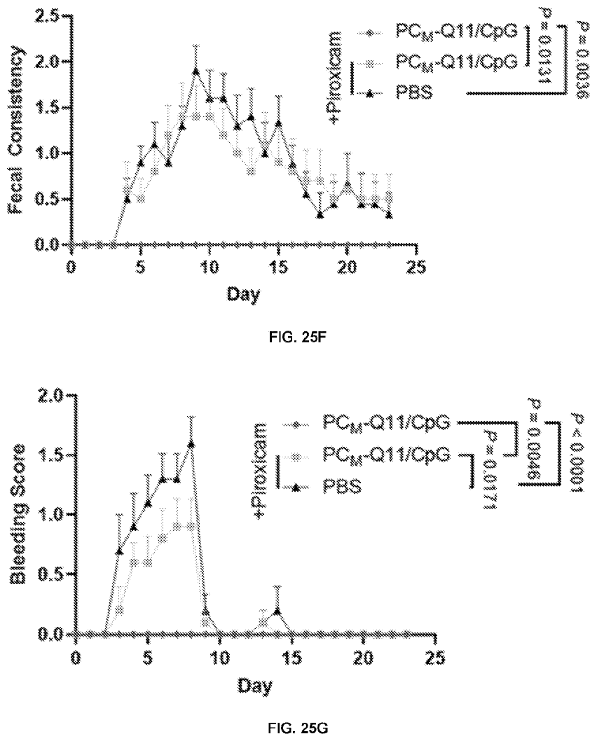

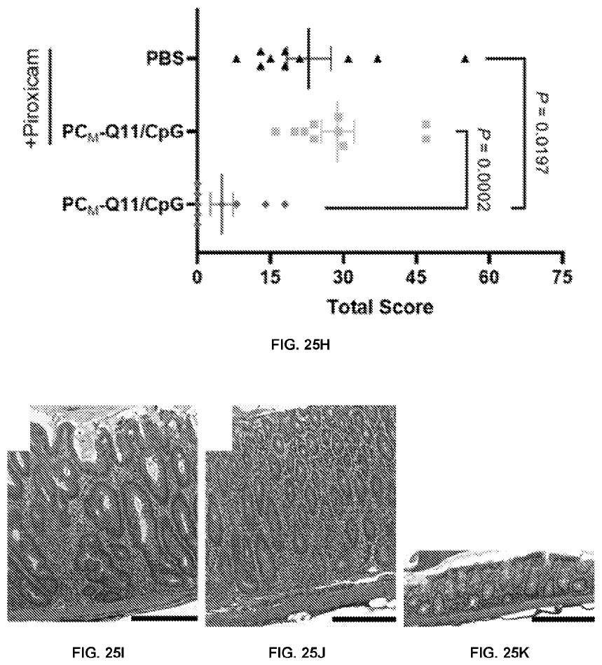

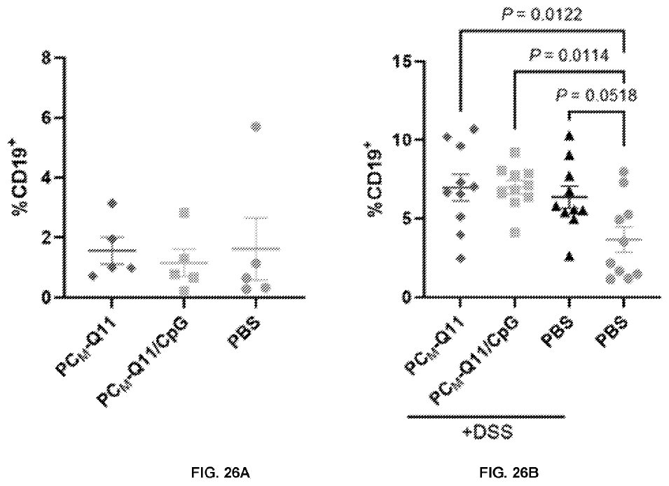

To determine the role IL-10 may have in the efficacy of PC immunizations and to evaluate the strategy in another colitis model, we immunized Il10^{-/-} mice with PC_{M}-Q11/CpG or PBS then triggered colitis by oral administration of the nonsteroidal anti-inflammatory drug (NSAID) piroxicam. Piroxicam induces epithelial cell apoptosis and allows bacterial influx into the mucosal tissue, triggering an antibacterial immune response that results in chronic colitis in the absence of IL-10⁶⁵ (model timepoints shown in FIG. 25A). Il10^{-/-} mice immunized with PC_{M}-Q11/CpG produced strong anti-PC IgG and IgM responses with similar IgG subclass biasing as in wild type mice (FIGS. 25B-25D). For the duration of the colitis model, there were no differences in weight loss or fecal consistency measures between piroxicam-administered groups (FIGS. 25E-25F). There was, however, a significant reduction in the occult bleeding score for PC_{M}-Q11/CpG immunized mice compared to disease controls (FIG. 25G). This reduction in bleeding was not associated with a decrease in the severity of colitis scored histologically on day 16 post-piroxicam in PC_{M}-Q11/CpG immunized compared to PBS-administered mice. Importantly, mice immunized with PC_{M}-Q11/CpG but not given piroxicam showed no colitis development at this time point (FIGS. 25H-25K). Together, this data showed while IL-10 is not necessary for the generation of strong anti-PC antibody responses, it may be important in the previously observed efficacy of PC_{M}-Q11/CpG immunizations in DSS-induced colitis.

### Example 10

### Effects of PC_{M}-Q11 Immunization and Colitis on B Cell Infiltrates in the Colon

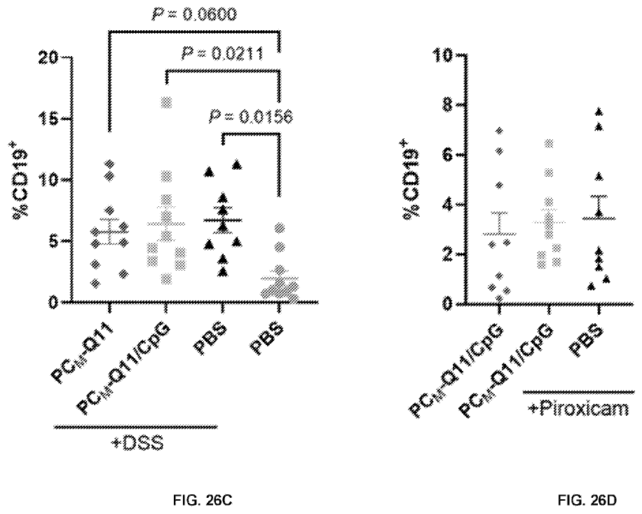

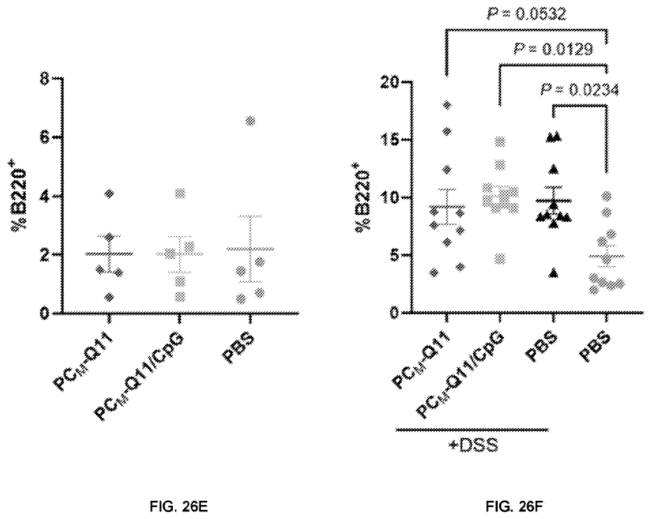

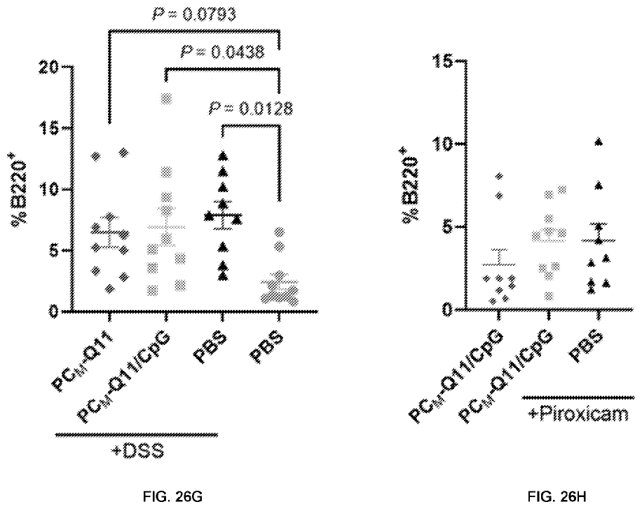

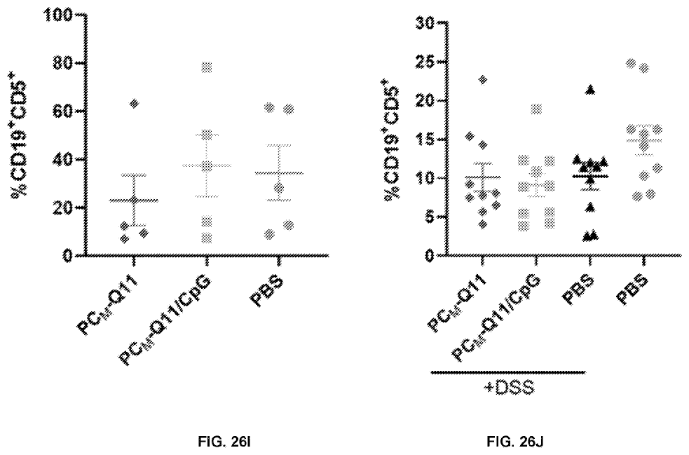

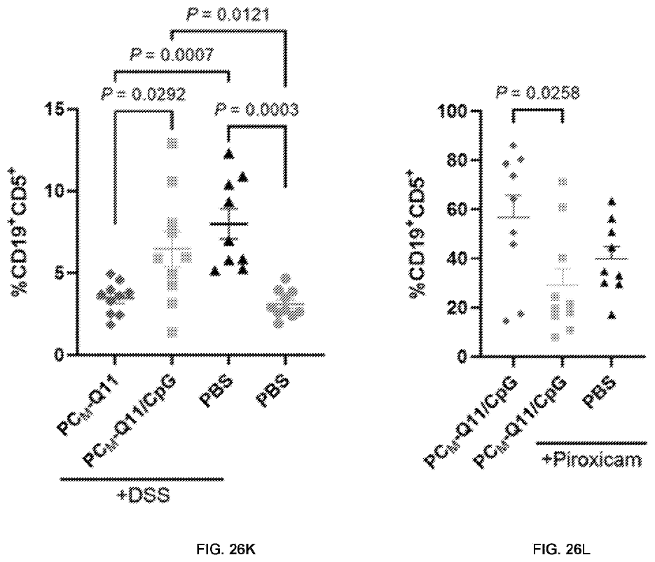

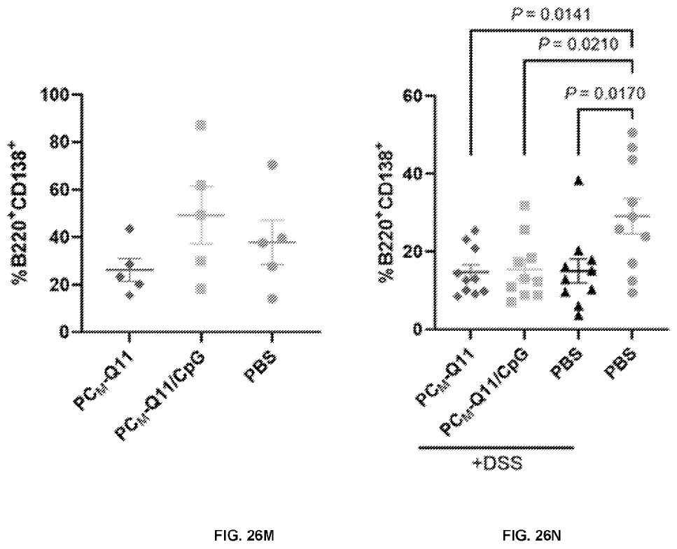

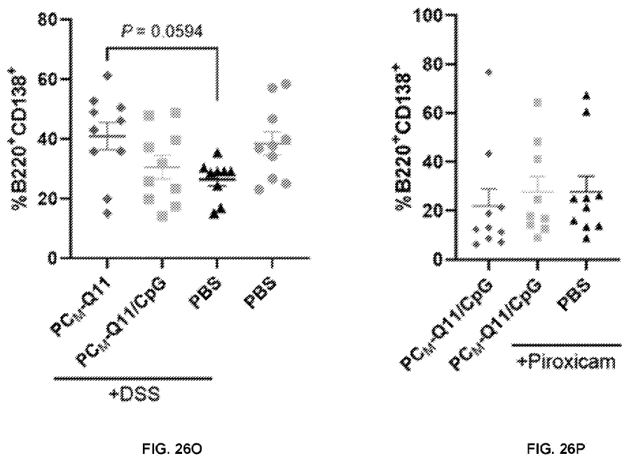

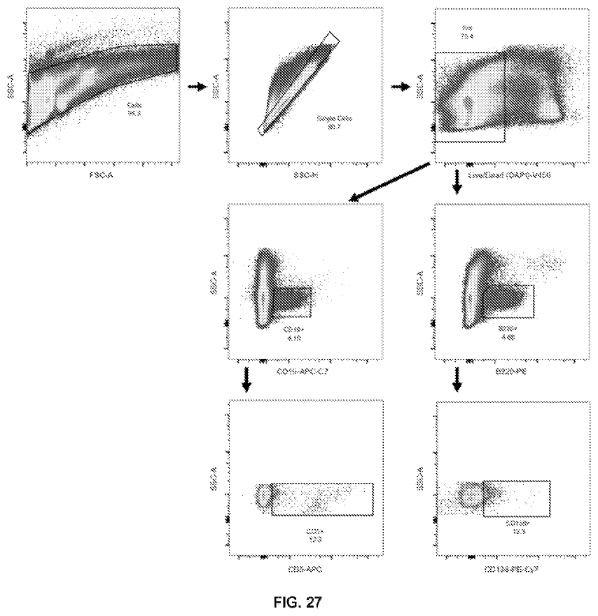

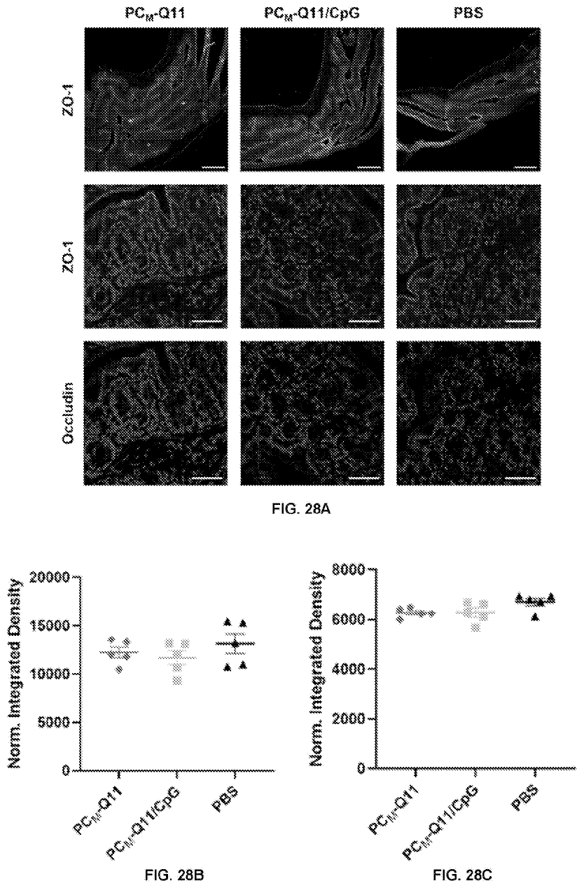

B1a cells are known to migrate and generate anti-inflammatory cytokines upon activation. While they mainly migrate to the spleen and lymph nodes, migration to the intestines with differentiation into antibody-secreting cells can also occur. Knowing that PC_{M}-Q11 nanofiber immunizations exhibit an affinity for B1a cells, we examined whether any therapeutic efficacy could be attributed to B1a cell recruitment from the peritoneal site of immunization to the colon. We analyzed the percentage of B cells (CD19⁺ or B220⁺), B1a cells (CD19⁺CD5⁺) and plasma cells (B220⁺CD138⁺) in mouse colons both after immunization and after the induction of colitis (FIGS. 26A-26P, representative flow cytometry shown in FIG. 27). We found that the percentage of colon B cells was greater for mice given DSS than healthy control mice but was not significantly different among groups in healthy immunized or Il10^{-/-} colitis mice (FIGS. 26A-26H). This result for DSS colitis was not unexpected, as the elevation of B-cell populations in inflammatory regions during colitis has been reported. Additionally, we did not observe any significant difference in the percentage of colonic B1a cells among groups in immunized mice or therapeutic DSS colitis mice (FIGS. 26I-26J). We did, however, notice a decrease in B1a cells in the chronic DSS colitis model for PC_{M}-Q11 immunized mice compared to other DSS-administered mice that resulted in similar B1a cell levels to healthy controls (FIG. 26K). On the other hand, in Il10^{-/-} mice, there was a trend towards more B1a cells in immunized healthy mice compared to piroxicam-administered mice (FIG. 26L). Finally, the only significant difference noted in plasma cell populations was in DSS colitis models. All DSS-administered mice in the therapeutic model had reduced plasma cells in the colon, but in chronic DSS colitis, PC_{M}-Q11 immunized mice had more plasma cells than disease controls, with these levels being more similar to healthy controls (FIGS. 26M-26P). Overall, our current findings of B cell populations after immunization and after colitis models did not strongly suggest that the presence of B1a cells or plasma cells in the colon contributes to therapeutic efficacy for anti-PC immunizations.

### Example 11

Evaluation of Colon Tight Junction Proteins Levels after PC_{M}-Q11 Immunization

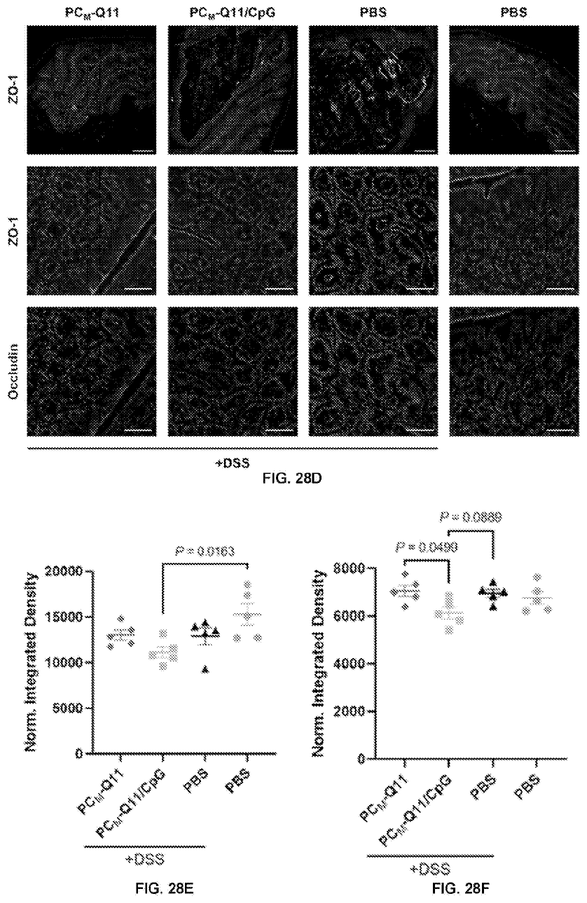

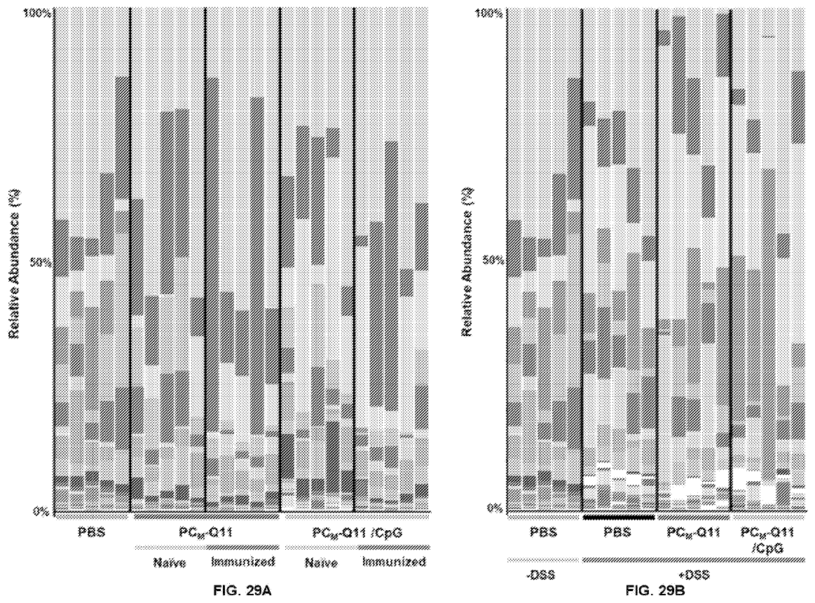

To ascertain if the observed reduction in disease severity that PC_{M}-Q11 immunizations provided in DSS colitis was due to effects of these immunizations on tight junction proteins in the colon, we quantified the levels of ZO-1 and occludin in colon sections from mice immunized with PC_{M}-Q11 and PC_{M}-Q11/CpG or PBS both after immunization and after chronic colitis (FIGS. 28A-28F). We found that ZO-1 and occludin levels were not significantly different after PC_{M}-Q11 or PC_{M}-Q11/CpG immunization when compared to PBS control mice (FIGS. 28A-28C). After chronic colitis, however, there was a reduction in ZO-1 levels in PC_{M}-Q11/CpG immunized mice compared to healthy controls (FIGS. 28D-28E). PC_{M}-Q11/CpG immunized mice also had slightly reduced occludin levels compared to other DSS-administered groups (FIGS. 28D-28F). It is important to note, however, that while there were slight differences in ZO-1 and occludin levels after chronic DSS colitis, these measurements only represent these protein levels where colonic epithelium was intact and not where ulceration caused by DSS itself locally destroys the mucosal barrier and markedly increases permeability (FIG. 28D).

### Example 12

### Pc_{M}-Q11 Immunizations Alter Gut Microbiome Diversity

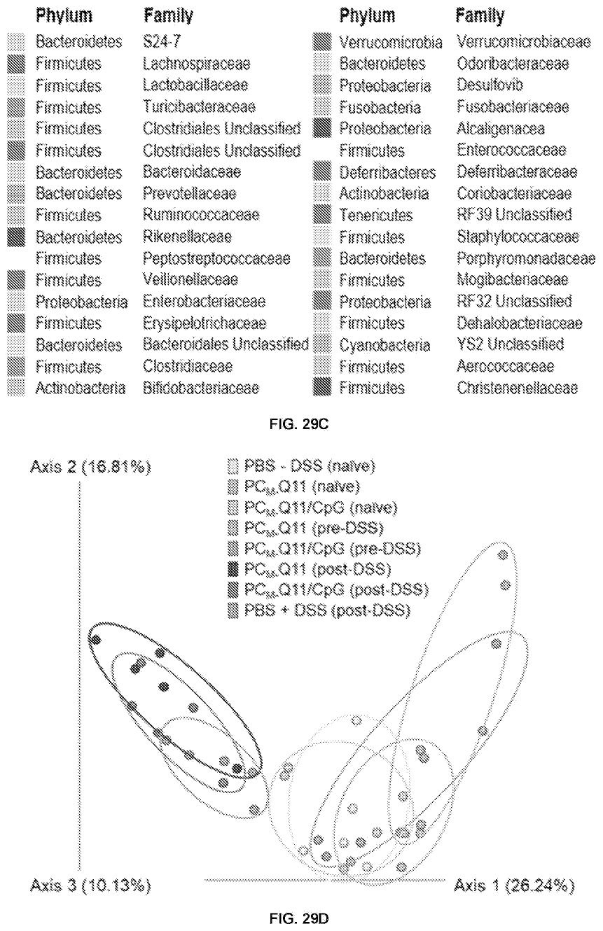

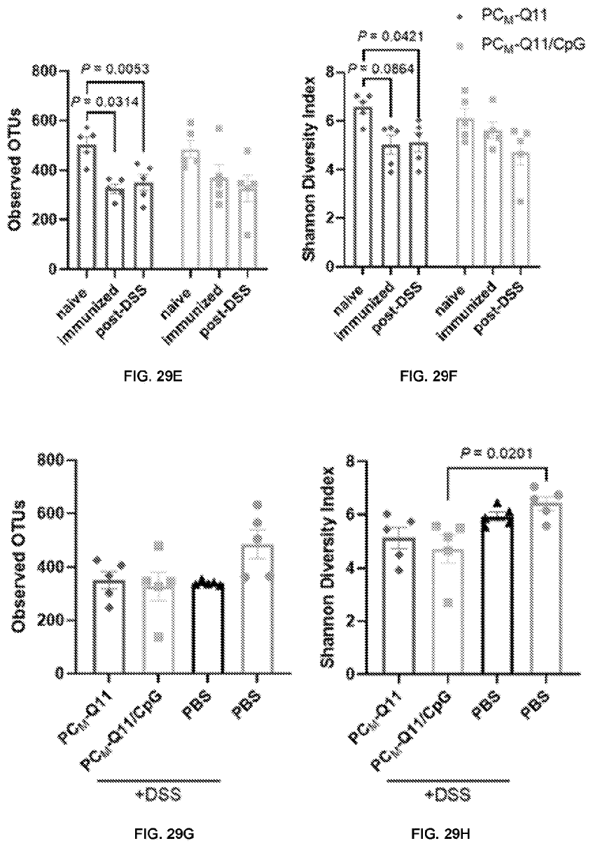

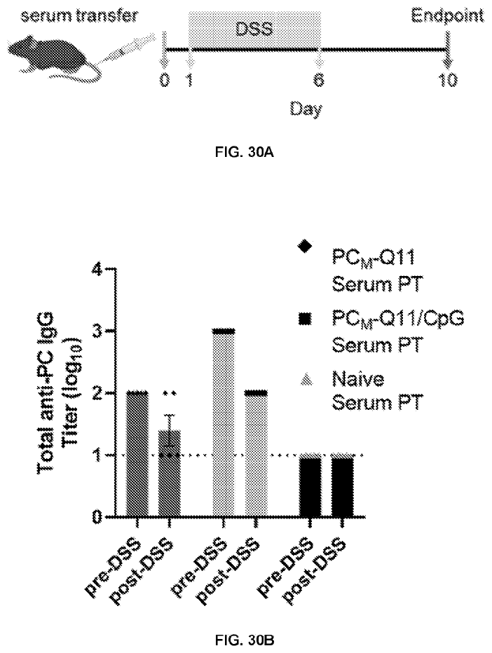

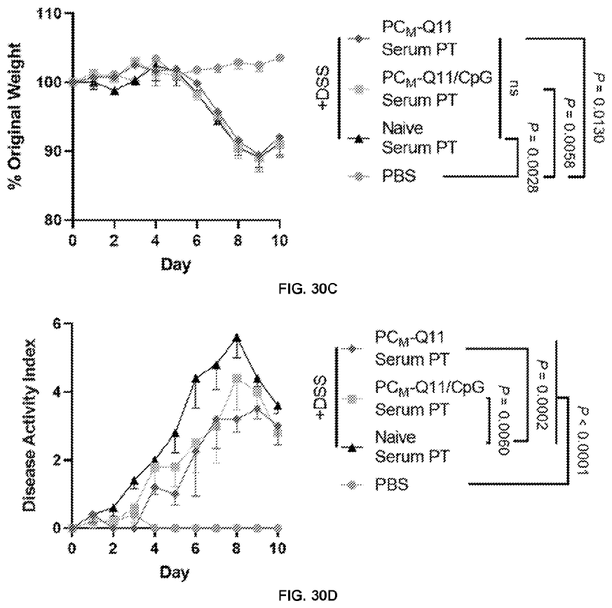

Changes in the gut microbiome have been associated with IBD⁶⁷ and are also seen in DSS-induced colitis. Given the presence of PC on bacteria, we sought to determine if immunization against PC altered the gut microbiome prior to the induction of chronic DSS colitis and to further examine how that change may play a role after colitis is induced. Sequencing of the V4 region of the 16S bacterial rRNA gene in fecal samples showed that immunization with PC_{M}-Q11 with or without CpG altered the relative abundance of different families of gut microbiota when compared to the same mice prior to immunization (FIG. 29A, FIG. 29C). We found that the gut microbiome was further modified by the induction of DSS colitis as evident by the changes in bacteria family relative abundance compared to healthy (PBS without DSS) control mice (FIGS. 29B-29C). Of note, bacteria of the Lactobacillaceae family were increased in immunized groups over healthy and diseased (PBS with DSS) controls. Bacteria in this family have been shown to be beneficial in both IBD and murine models of colitis. We further identified gut microbiome modification when measuring beta diversity via a Jaccard Emperor plot. Clusters of naïve groups and DSS-administered groups showed distinct profiles, whereas immunized groups, while also distinct from DSS-administered groups, showed some similarities to naïve groups (FIG. 29D). Bacterial richness, as measured by observed operational taxonomic units (OTUs), was decreased for immunized mice both with and without CpG (FIG. 29E). The Shannon diversity index was similarly reduced for immunized mice (FIG. 29F). Additionally, comparable decreases in diversity from healthy mice were seen for both immunized groups and DSS disease controls based on both observed OTUs and Shannon diversity indices (FIGS. 29G-29H). While gut microbiomes more similar to healthy mice in both level of diversity and taxonomic resemblance are typically regarded as the goal for treatments in DSS colitis models (Nagalingam, N. A., et al. Inflamm. Bowel Dis. 2011, 17, 917-926, incorporated by reference), using those criteria here was confounded by the changes in the gut microbiome resulting from PC_{M}-Q11 immunizations alone. Despite the decreased microbiome diversity seen in immunized mice before and after DSS-induced colitis, their lessened disease severity was apparent. It may be further studied how these gut microbiome differences could have potentially contributed to this therapeutic effect.

### Example 13

PC-Immune Serum Offers Some Protection when Passively Transferred Before Colitis and Binds Apoptotic Cells

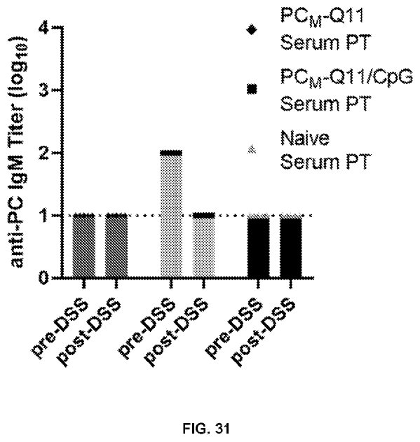

Seeking to clarify the therapeutic mechanism of nanofiber immunizations in colitis, we next determined the extent to which PC_{M}-Q11's affinity for B1a cells or its ability to generate anti-PC antibodies contributed to its therapeutic efficacy. Naïve mice were administered via tail vain injection a passive transfer (PT) of sera from PC_{M}-Q11 or PC_{M}-Q11/CpG immunized mice, or naïve sera from PBS-injected mice. One cycle of DSS-induced colitis was then initiated (FIG. 30A). We confirmed anti-PC IgG and IgM antibody titers after PT before inducing colitis (FIG. 30B and FIG. 31). While serum PT of PC_{M}-Q11 or PC_{M}-Q11/CpG sera did not have an effect on weight loss, it did significantly reduce DAI scores (FIGS. 30C-30D), indicating that the induced anti-PC antibodies likely offered some protection against severe colitis. Consistent with this finding, PC-immune serum also conferred protection against colon shortening compared with naïve serum PT controls, with PC_{M}-Q11/CpG serum PT mice having significantly longer colons (FIG. 30E). However, this protection did not extend to all measurements of disease, as no difference in colon levels of IL-10 nor significant reduction in bacteria in the spleen was observed between diseased groups (FIGS. 30F-30G). Given the partial protection conferred by transferred serum, it is possible that the number of local or systemic antibodies was not as great nor as continuously produced as in immunized mice. It is also possible that serum factors such as antibodies against PC may not be solely responsible for the therapeutic efficacy of PC_{M}-Q11 immunizations.

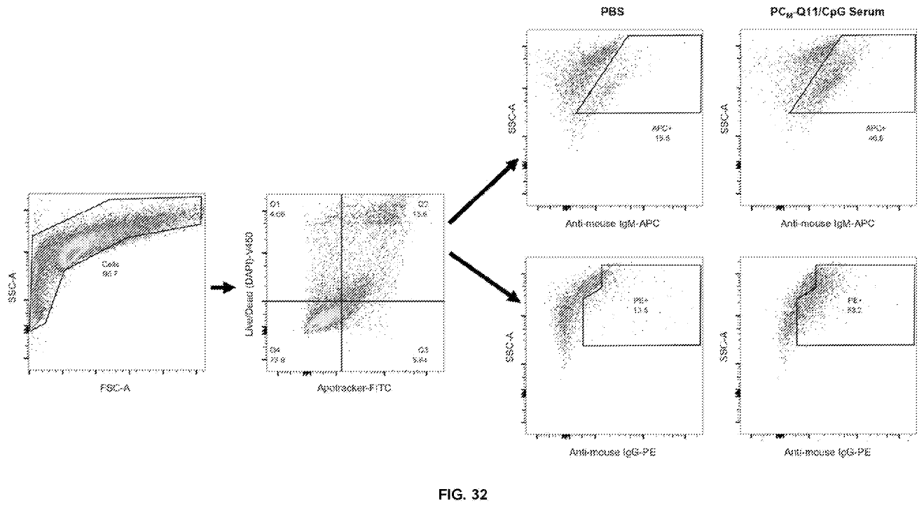

Furthermore, since one native function of anti-PC natural antibodies is to facilitate the clearance of apoptotic debris, we investigated the ability of antisera from immunized mice to bind apoptotic cells in vitro. Serum from PC_{M}-Q11/CpG-immunized mice contained elevated IgM and IgG that bound to apoptotic cells (FIGS. 30H-30J, representative flow cytometry in FIG. 32), and serum from PC_{M}-Q11-immunized mice, contained elevated IgG against apoptotic cells (FIGS. 30I-30J). This corresponded with previous findings where mice immunized with CpG had higher IgM levels than those immunized without adjuvant.

Collectively, these findings indicated that anti-PC active immunotherapies produce responses capable of improving colitis disease severity that could have implications as a long-term therapy for IBD.

### Example 14

### Oral Administration

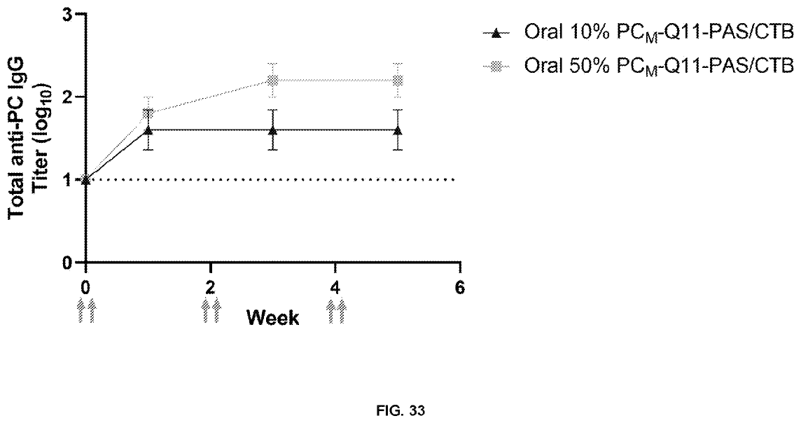

We examined PASylation of PC_{M}-Q11 nanofibers by including the muco-inert peptide sequence PAS (H₂N-ASPAAPAPASPAAPAPSAPA-NH₂) that enables oral vaccination, and results are shown in FIG. 33. Mice were immunized via oral gavage on two consecutive days every two weeks (indicated by grey arrows on graph) with 2 mM nanofiber formulations consisting of either 0.2 mM (10%) or 1 mM (50%) PC_{M}-Q11-PAS coassembled with PAS-Q11 (1.75 or 0.95 mM respectively) and the T-cell epitope PADRE-Q11 (0.05 mM). 10 μg of cholera toxin B subunit (CTB) was also included in oral vaccine formulations. Results indicated that mice given 50% PC_{M}-Q11-PAS raised anti-PC IgG responses comparable to intraperitoneal (i.p.) immunization without adjuvant.

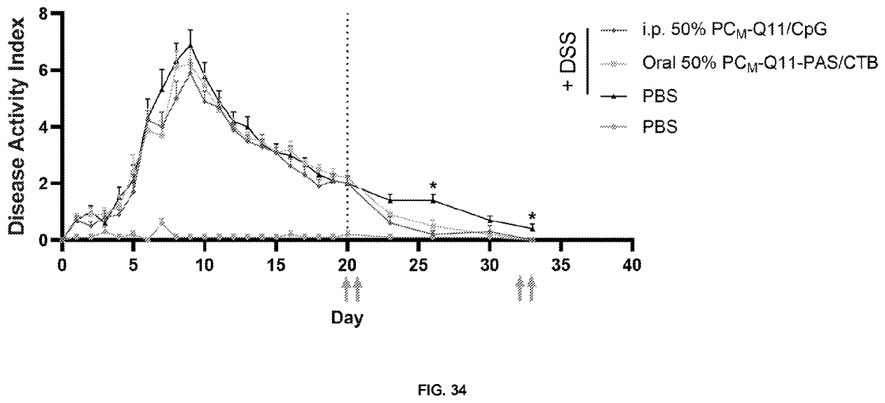

In an ongoing therapeutic model of DSS-induced colitis, mice were given 2% DSS in drinking water for 5 days and then allowed to recover for 15 days before being immunized either i.p. with 50% PC_{M}-Q11 and CpG adjuvant (on days 20 and 32) or orally with 50% PC_{M}-Q11-PAS with CTB adjuvant (on days 20 & 21 and 32 & 33). Results are shown in FIG. 34. Mice from immunized groups had significantly lower disease activity index scores on day 26 after just the initial immunizations and on day 33. Statistical analysis was determined by one-way ANOVA on day 26 or 33 with Tukey's multiple comparison test and *P<0.05.

### Example 15

### Discussion

IBD is a debilitating, chronic disease for which a long-term and widely efficacious therapy does not exist. While standard-of-care monoclonal antibody therapeutics are effective in some patients, they still present drawbacks including high primary and secondary patient non-response rates and an increased risk of severe infections. Active immunotherapies in which vaccination is used to induce endogenous antibodies against therapeutic targets are a promising solution. Presented herein is the first example of an active immunotherapy using phosphorylcholine for the treatment of IBD. By using natural anti-PC antibodies which are inherently anti-inflammatory and important for the clearance of apoptotic cells, bacteria and other pathogens as a basis for our design, we demonstrated that self-assembling peptide nanofibers bearing PC epitopes can be used to induce robust multi-class anti-PC antibody responses for therapeutic efficacy in colitis models. Others have pursued vaccination against different targets, including the cytokines IL-12/IL-23 and TGF-β1 and the pro-angiogenic factor, EMMPRIN, for IBD. As with passive immunization with monoclonal antibody biologics against such targets, however, the risk of impairing the beneficial, non-disease-causing effects, such as aiding in the prevention of severe infections, when depleting these multipurpose molecules is high and also not easily reversible when a long-lasting antibody response and breaks in T-cell tolerance are achieved. Consequently, when comparing immunization against PC with these three targets for the treatment of IBD, there are considerable advantages of building upon a natural anti-inflammatory antibody response as opposed to the risks associated with generating antibodies against multifaceted targets.

Previously investigated vaccines against PC consisted of PC attached to carrier proteins, most commonly Keyhole Limpet Hemocyanin (KLH), and are generally in the context of bacterial infections. PC-KLH immunization has also been investigated in mouse models of atherosclerosis, including i.p. vaccination with PC-KLH/CpG adjuvant and intranasal immunization with PC-KLH/DNA-based adjuvant. Despite only moderate anti-PC antibody production, both vaccines lessened atherosclerotic lesion severity, illustrating the promise of anti-PC therapies in an inflammatory model. Carrier protein vaccines, however, largely lacked the ability for fine control over B-cell epitope spacing, contain unremovable exogenous T-cell epitopes, and can be inherently inflammatory or require adjuvants that skew the immune phenotype. The importance of B-cell epitope multivalency and spacing in immune cell activation and the generation of robust cellular and humoral immune responses in the absence of adjuvant has been clearly demonstrated previously. In this work, we found that altering PC multivalency enhanced B1a cell selectivity and increased anti-PC antibody responses. Furthermore, it has been shown that the inclusion of an exogenous T-cell epitope and its ratio to the B-cell epitope is critical in modulating the strength and phenotype of the immune response, especially against self-molecules. This is further illustrated here, where the addition of the T-cell epitope PADRE enhanced anti-PC immune responses. Lastly, in many autoimmune diseases, inflammation is central to the pathogenesis. It is therefore generally favorable for vaccinations against such diseases to not increase inflammation either themselves or through the addition of necessary adjuvant. PC-carrier protein vaccines have largely required the addition of adjuvant to induce responses capable of therapeutic efficacy. In contrast, PC_{M}-Q11 vaccination generated similar reductions in colitis disease severity irrespective of CpG adjuvant incorporation. Collectively, we demonstrated the ability of modular Q11 nanofibers to generate tailored anti-PC immune responses according to these parameters, which are part of designing long-lived and broadly effective active immunotherapies.

In addition to PC-protein vaccines, PC attached to tuftsin, a tetrapeptide capable of stimulating innate immune cells, has been investigated as both a subcutaneously- and orally-delivered drug in mouse models of autoimmune inflammatory diseases including systemic lupus erythematosus. PC-tuftsin has also been investigated DSS-induced colitis models, where it was given orally every day for 11 days, starting before DSS administration, and extending through recovery in an acute model and was administered daily for 15 days at the conclusion of a chronic model. While modest efficacy was observed, the mechanism and longevity of these anti-inflammatory responses were not fully investigated. Therefore, it is unknown what role an adaptive immune response to PC may have played in the therapeutic efficacy of these treatments, or if less frequent dosing would have the same impact on disease severity. In contrast, i.p. immunization with PC_{M}-Q11 induced an adaptive anti-PC antibody immune response capable of protection in a 30-day chronic DSS-induced colitis model with 3 prophylactic vaccine doses. It was also shown to be effective as a 3-dose therapeutic vaccine between DSS colitis cycles. While these vaccination results show promise in two different disease scenarios, any envisioned clinical IBD setting may initiate treatment at various stages of disease. Therefore, multiple vaccination schedules, including immunization concurrent with colitis induction and longer-term studies with delayed colitis induction, may be examined.

We selected the TLR-9 agonist, CpG, to adjuvant our anti-PC active immunotherapy due to its demonstrated effects against bacterial infections and the protective role that TLR-9 activation plays in colitis in mice as well as humans. When analyzing the effects of immunizing with PC_{M}-Q11 versus PC_{M}-Q11/CpG, we observed an increased anti-PC antibody response with the addition of CpG that included higher levels of IgM and IgG3 as well as the more inflammatory IgG2b and IgG2c subclasses and a Th1-biased T-cell response. While this response with CpG was anticipated, the fact that both formulations had the same therapeutic efficacy was surprising. The exact rationale for this observation was not known, but it is possible that the inflammation and Th1 T-cell response induced by CpG is counteracted by the induction of anti-PC IgM and IgG3 as well as TLR-9 activation for an overall positive phenotype for reducing colitis disease severity. Additionally, the different IgG subclasses generated by each vaccine act on different Fc receptors with varying roles. Immunization with PC_{M}-Q11 alone elicited predominantly IgG1. Mouse IgG1 only binds FcRn, FcγRIIB, and FcγRIII, all of which have been tied to anti-inflammatory effects. While these receptors are also bound by IgG2b and IgG2c, these subclasses are associated with other activating Fc receptors on macrophages and neutrophils leading to inflammatory cytokine production and are similar to human IgG1 in that they induce potent complement activation. Regardless of the exact cause of the observed similar therapeutic efficacies of both PC_{M}-Q11 and PC_{M}-Q11/CpG, however, immunizations without adjuvant are favorable for human translation as they lessen side-effects, and CpG adjuvants have differing effects in mice and humans, making the ability to use PC_{M}-Q11 without adjuvant advantageous.

Additionally, the role of the host gut microbiome in IBD is complex and not yet completely understood. Dysbiosis that lowers taxonomic diversity of gut microbiota is common in patients with both Crohn's disease and ulcerative colitis, as well as in animal studies of IBD; however, differing changes in specific bacteria populations or potential specific bacterial targets have been reported. This includes reports that bacteria in the Lactobacillaceae family, which are increased in PC_{M}-Q11 immunized mice after colitis, are protective in IBD. Additionally, the creation of mucosal polyreactive IgA and IgM antibodies that help regulate the microbiome is thought to be driven by natural antibody-producing intestinal B cells. Here, we found that the generation of anti-PC antibodies affects the gut microbiome, decreasing bacterial diversity after immunization even before DSS-induced colitis. Microbiome dysbiosis compared to healthy controls was further seen at the end of the DSS-induced chronic colitis model. The conventional goal when comparing the microbiomes of colitis-stricken and healthy mice is to see similar microbiome composition. PC_{M}-Q11 immunization alone had already altered the diversity, and PC_{M}-Q11 immunized mice had less severe disease. Thus anti-PC effects on the diversity of the microbiome may be examiner further and the underlying mechanisms of how these changes could be beneficial in colitis.

In this work, we demonstrated i.p. vaccination as an optimal route for inducing an anti-PC antibody response. i.p. immunization was advantageous for B1a cell activation and was an ideal route important for generating baseline principles for effective anti-PC immunization in colitis. PC_{M}-Q11 immunization via other routes that have more translatable potential may be examined, including the intranasal route. Additionally, B1a cells are prominent in the pleural cavity, and intranasal immunization with PC-KLH and a DNA-based adjuvant has been shown to increase B1a cell populations in not only the pleural cavity but also more systemically in the spleen. Therefore, intranasal immunization with PC_{M}-Q11 may allow for potent B1a cell activation in addition to the generation of mucosal anti-PC antibodies in the intestines, potentially improving therapeutic efficacy at the disease site.

In total, our experiments suggested that the observed efficacy of PC_{M}-Q11 immunizations can be significantly attributed to induced anti-PC antibody response and is likely dependent on IL-10. Other factors may also contribute and may be studied. PC-immune sera transferred into naïve mice reduced DSS colitis disease severity, and that therapeutic efficacy was reduced in an Il10^{-/-} murine model of colitis. IL-10 is an important immunosuppressive cytokine with many important roles including mitigating inflammatory immune responses in the gut. IL-10 may be necessary for anti-PC antibodies to clear their targets in a non-inflammatory manner. IL-10 secretion is also associated with B1a cells and natural antibody production, however, we did not find strong evidence of an increased presence of B1a cells or plasma cells in the colon. Regulatory B10 cells are phenotypically similar to B1a cells, sharing several overlapping surface markers including CD5, and are also known to secrete high levels of IL-10. Therefore, the potential role of IL-10 secreting B10 cells in PC_{M}-Q11 vaccination efficacy may be studied further. Additionally, previous studies have shown that there is a delicate interplay between regulatory B and T cells involved in maintaining gut homeostasis and offering protection against colitis. We have shown that CD4⁺ T cells are important for augmenting PC_{M}-Q11 immunization-induced anti-PC antibody production. Other T-cell subsets, however, have been shown to be both beneficial and pathogenic in patients with IBD as well as murine models of colitis. Here, we conducted a shorter 10-day DSS-induced colitis model. At this time point, DSS colitis better models the innate immune factors contributing to colitis, as it has been shown that severe combined immunodeficiency (SCID) Rag^{-/-} mice will still develop DSS-induced colitis in this time frame. We also conducted a longer, chronic DSS-induced colitis model. In chronic DSS-induced colitis models, defects in lymphocyte function have been shown to play a role, with potential pathogenic roles of T cells or a reduction in beneficial T-cell subsets seen. Given the observed efficacy of PC_{M}-Q11 immunizations in multiple DSS colitis models where T cell involvement varies, the specific and time-dependent roles of T cells and what effect, if any, they have on PC_{M}-Q11 immunization efficacy may be studied further.

Furthermore, while we can attribute the observed efficacy of PC_{M}-Q11 immunizations to the induced anti-PC antibody response, our investigations into the effects of PC immunizations on intestinal barrier integrity in DSS-induced colitis showed that there were only small changes in the expression of the tight junction proteins ZO-1 and occludin as well as FITC-dextran leakage into the serum. Histological evaluation of colon sections also showed either slight or no trends in improvement in colonic epithelial damage at the experimental endpoints, 5 days after DSS administration ceased. The lack of significant histological improvements seen at this timepoint, however, could be attributed to the fact that histological changes can happen more slowly than and do not always correlate with clinical symptom improvements. Conversely, bacterial levels in the spleen, often used as a marker for colon epithelial damage, were decreased, conflicting our other findings on epithelial barrier disruption and suggesting immunization may alternatively affect bacterial clearance. PC_{M}-Q11 immunizations were also shown to reduce microbiome diversity, indicating that the induced anti-PC antibodies may bind to and clear certain bacteria that display PC epitopes. Combined with the reduced bacterial spread, these findings suggested that PC_{M}-Q11 may not strengthen intestinal barrier function per se but rather that some leaked bacteria are bound by induced anti-PC antibodies and cleared before they can escape to other tissues. We were also able to demonstrate that anti-PC antibodies induced by PC_{M}-Q11 bind apoptotic colon epithelial cells in vitro, providing a method through which these antibodies could be clearing injured host cells. Altogether, these results suggested that PC_{M}-Q11 immunizations act through induced anti-PC antibodies to clear bacterial and autologous targets in a non-inflammatory manner requiring IL-10.

In summary, we developed a unique phosphorylcholine-targeted active immunotherapy for inflammatory bowel disease. We designed tunable Q11 peptide nanofibers generating immune responses against the small molecule epitope, PC, and we evaluated the importance of conjugate structure and multivalent epitope display for B1a cell selectivity and the generation of anti-PC antibody responses. We also showed control over the elicited immune phenotype by including a T-cell epitope and supplementing with adjuvant. Additionally, we found that immunization with PC_{M}-Q11, regardless of CpG adjuvant inclusion, reduced colitis severity when administered both prophylactically and therapeutically in DSS-induced models of colitis, lessening disease severity and demonstrating the robustness of this therapy. Furthermore, the induced anti-PC antibody response was a considerable component of the exhibited efficacy.

There are currently no therapies for IBD that are long-lasting and broadly efficacious. Available therapies also raise concerns over the development of anti-drug antibodies and increase the risk of infection. The development of an active immunotherapy for IBD may provide a new treatment option for this disease. Peptide nanofibers can generate durable and effective therapeutic anti-PC immune responses. Building on natural antibody responses, anti-PC immunizations may be an alternative treatment.

### Example 16

### Testing of Additional Self-Assembling Peptides

A PC-conjugate peptide will be generated, including at least one PC molecule and a self-assembling peptide comprising the amino acid sequence of QARILEADAEILRAYARILEAHAEILRAQ (Coil29, SEQ ID NO: 6). The PC-conjugate peptide will be similar to the peptide detailed in Example 2, but with the different self-assembling peptide. A PADRE-conjugate peptide, as well as a plain self-assembling peptide, will be generated with the same self-assembling peptide. A nanofiber will be formed, including alpha-helices. The nanofiber will be administered to mice as detailed in Examples 3-14. The PC-conjugate peptide will reduce and/or ameliorate colitis.

### Example 17

### Treatment of Inflammatory Disease

PC-conjugate peptides as detailed in Examples 1-16 will be administered to mice, tissue culture, or other animal model. The PC-conjugate peptides will ameliorate, reduce the symptoms of, and/or treat an inflammatory disease selected from inflammatory bowel disease (IBD), ulcerative colitis, Crohn's disease, rheumatoid arthritis, psoriasis, and cirrhosis.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A phosphorylcholine (PC)-conjugate peptide comprising: (i) a self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and (ii) at least one PC molecule conjugated to a terminus of the self-assembling peptide.

Clause 2. The PC-conjugate peptide of clause 1, wherein each self-assembling peptide forms a beta sheet and comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn).

Clause 3. The PC-conjugate peptide according to clause 1 or 2, wherein the self-assembling peptide comprises the sequence QQKFQFQFEQQ (SEQ ID NO: 31) or Ac-QQKFQFQFEQQ-NH₂ (SEQ ID NO: 99).

Clause 4. The PC-conjugate peptide of clause 1, wherein each self-assembling peptide forms an alpha-helix and comprises a polypeptide having an amino acid sequence of bXXXb (SEQ ID NO: 1), wherein X is independently any amino acid and b is independently any positively charged amino acid.

Clause 5. The PC-conjugate peptide of clause 1 or 4, wherein b is independently selected from Arg and Lys.

Clause 6. The PC-conjugate peptide of clause 1, 4, or 5, wherein bXXXb (SEQ ID NO: 1) is RAYAR (SEQ ID NO: 2) or KAYAK (SEQ ID NO: 3).

Clause 7. The PC-conjugate peptide of any one of clauses 1 and 4-6, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

Clause 8. The PC-conjugate peptide of clause 7, wherein the self-assembling peptide comprises an amino acid sequence selected from

```
  (Coil29, SEQ ID NO: 6)
QARILEADAEILRAYARILEAHAEILRAQ,
or
  (SEQ ID NO: 7)
QAKILEADAEILKAYAKILEAHAEILKAQ,
or
(SEQ ID NO: 8)
ADAEILRAYARILEAHAEILRAQ,
or
  (SEQ ID NO: 22)
Ac-QARILEADAEILRAYARILEAHAEILRAQ-NH₂,
or
  (SEQ ID NO: 23)
Ac-QAKILEADAEILKAYAKILEAHAEILKAQ-NH₂,
or
  (SEQ ID NO: 24)
Ac-ADAEILRAYARILEAHAEILRAQ-NH₂.
```

Clause 9. The PC-conjugate peptide of any one of clauses 1-8, further comprising: (iii) a linker between the at least one PC molecule and the self-assembling peptide.

Clause 10. The PC-conjugate peptide of clause 9, wherein the linker comprises an amino acid sequence selected from SEQ ID NO: 25 ((Ser-Gly)₂), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), SEQ ID NO: 29 (EAAAK)₂, and SEQ ID NO: 30 (GGAAY).

Clause 11. The PC-conjugate peptide of clause 10, wherein the linker comprises (Ser-Gly)₂ (SEQ ID NO: 25).

Clause 12. The PC-conjugate peptide of any one of clauses 9-11, wherein each PC molecule is attached to the linker via a Cys residue or an Asn residue.

Clause 13. The PC-conjugate peptide of any one of clauses 1-12, wherein the at least one PC molecule is attached to the C-terminus or the N-terminus of the self-assembling peptide.

Clause 14. The PC-conjugate peptide of any one of clauses 1-13, wherein 1 to 10 PC molecules are attached to the C-terminus or the N-terminus of the self-assembling peptide.

Clause 15. The PC-conjugate peptide of clause 14, wherein each PC molecule is independently attached to the linker via a Cys residue or an Asn residue.

Clause 16. The PC-conjugate peptide of any one of clauses 1-15, wherein 1 to 10 PC molecules are attached to the C-terminus or the N-terminus of the self-assembling peptide via a single linker.

Clause 17. The PC-conjugate peptide of clause 16, wherein 1 to 4 PC molecules are attached to the C-terminus or the N-terminus of the self-assembling peptide.

Clause 18. The PC-conjugate peptide of clause 17, wherein 4 PC molecules are attached to the C-terminus or the N-terminus of the self-assembling peptide.

Clause 19. The PC-conjugate peptide of clause 18, comprising the sequence of (PC molecule)₄-CCCCSGSG-QQKFQFQFEQQ-NH₂ SEQ ID NO: 34), wherein each one of the four PC molecules is attached to a different Cys in the linker CCCCSGSG (SEQ ID NO: 26).

Clause 20. The PC-conjugate peptide of any one of clauses 1-19, wherein each PC molecule is independently selected from C₅H₁₄NO₄P and C₁₁H₂₂NO₆P.

Clause 21. The conjugate peptide of any one of clauses 1-20, wherein the PC-conjugate peptide comprises a sequence selected from C₅H₁₄NO₄P-SGSG-QQKFQFQFEQQ-NH₂ (pa-PC-Q11, SEQ ID NO: 32), or C₁₁H₂₂NO₆P-CSGSG-QQKFQFQFEQQ-NH₂ (PC-Q11, SEQ ID NO: 33), or (C₁₁H₂₂NO₆P)ₙ-CCCCSGSG-QQKFQFQFEQQ-NH₂ (PC_{M}-Q11, SEQ ID NO: 34) wherein n is an integer selected from 1, 2, 3, and 4).

Clause 22. The PC-conjugate peptide of any one of clauses 1-21, further comprising a PAS peptide of SEQ ID NO: 35 or 36 conjugated to the self-assembling peptide at the opposite terminus from wherein the PC molecule is attached.

Clause 23. A nanofiber comprising a plurality of the PC-conjugate peptide of any one of clauses 1-22, wherein the conjugate peptide self-assembles into the nanofiber.

Clause 24. A nanofiber comprising: (i) at least one PC-conjugate peptide of any one of clauses 1-22; and (ii) at least one PADRE-conjugate peptide comprising: a self-assembling peptide, wherein each self-assembling peptide independently comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and at least one PADRE molecule, wherein at least one self-assembling peptide is conjugated to at least one PADRE molecule, and wherein the PADRE molecule comprises a polypeptide having the amino acid sequence of aKXVAAWTLKAa (SEQ ID NO: 18, wherein “X” comprises cyclohexylalanine and “a” comprises D-alanine).

Clause 25. The nanofiber of clause 24, wherein the PADRE-conjugate peptide further comprises a linker between the PADRE molecule and the self-assembling peptide.

Clause 26. The nanofiber of clause 25, wherein the linker comprises an amino acid sequence selected from SEQ ID NO: 25 ((Ser-Gly)₂), SEQ ID NO: 26 (CCCCSGSG), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), and SEQ ID NO: 29 (EAAAK)₂, and SEQ ID NO: 30 (GGAAY).

Clause 27. The nanofiber of any one of clauses 24-26, wherein the at least one PADRE-conjugate peptide comprises the sequence of NH₂-aKXVAAWTLKAa-SGSG-QQKFQFQFEQQ-NH₂ (PADRE-Q11, SEQ ID NO: 37).

Clause 28. The nanofiber of any one of clauses 24-27, wherein the cyclohexylalanine comprises D-alanine.

Clause 29. The nanofiber of any one of clauses 24-28, further comprising: (iv) a plain self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid), without any PC molecules attached thereto.

Clause 30. The nanofiber of any one of clauses 24-29, wherein the nanofiber comprises a combination of 10 to 10,000, or 100 to 10,000 peptides comprising PC-conjugate peptides and PADRE-conjugate peptides.

Clause 31. The nanofiber of clause 29, wherein the nanofiber comprises a combination of 10 to 10,000, or 100 to 10,000 peptides comprising PC-conjugate peptides, PADRE-conjugate peptides, and plain self-assembling peptides.

Clause 32. The nanofiber of any one of clauses 24-31, wherein at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are PC-conjugate peptides.

Clause 33. The nanofiber of any one of clauses 24-32, wherein at least about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the peptides in the nanofiber are PADRE-conjugate peptides.

Clause 34. The nanofiber of any one of clauses 29-33, wherein at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides.

Clause 35. The nanofiber of any one of clauses 29-34, wherein about 50% of the peptides in the nanofiber are PC-conjugate peptides, about 2.5% of the peptides are PADRE-conjugate peptides, and about 47.5% of the peptides are plain self-assembling peptides.

Clause 36. The nanofiber of any one of clauses 24-35, wherein the PC-peptide conjugate and the PADRE-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

Clause 37. The nanofiber of any one of clauses 24-36, wherein the self-assembling peptide forms a fibril including beta-sheet structures.

Clause 38. The nanofiber of any one of clauses 24-36, wherein the self-assembling peptide forms a fibril having a coiled coil structure.

Clause 39. The nanofiber of any one of clauses 24-36 and 38, wherein the self-assembling peptide forms a fibril having a structure of a helical filament formed around a central axis.

Clause 40. The nanofiber of clause 39, wherein the N-terminus of each self-assembling peptide is positioned at the exterior of the helical filament.

Clause 41. The nanofiber of any one of clauses 24-40, wherein the PC molecules are exposed on the exterior surface of the nanofiber.

Clause 42. The nanofiber of any one of clauses 24-41, wherein the nanofiber is about 5-20 nm in width.

Clause 43. The nanofiber of any one of clauses 24-42, wherein the nanofiber is about 100 nm to 1 μm, 100 nm to 2 μm, 100 nm to 3 μm, 100 nm to 4 μm, or 100 nm to 5 μm in length.

Clause 44. A phosphorylcholine (PC)-conjugate peptide comprising: (i) a self-assembling peptide, wherein the self-assembling peptide comprises a polypeptide having an amino acid sequence selected from VEVKVEVKV (SEQ ID NO: 65), VEVKVEVKVEVK (SEQ ID NO: 66), VVVAAAEEE (SEQ ID NO: 67), VEVEVEVEVEVEVEVEVEVE (SEQ ID NO: 68), CGNKRTRGC (SEQ ID NO: 69), VKVKVKVKVDPPTKVEVKVKV (SEQ ID NO: 70), LRKKLGKA (SEQ ID NO: 71), VVVVVVKK (SEQ ID NO: 72), AEAKAEAKAEAKAEAK (SEQ ID NO: 47), AEAKAEAK (SEQ ID NO: 73), AEAEAEAEAKAK (SEQ ID NO: 74), AEAEAKAK (SEQ ID NO: 75), AEAEAKAKAEAEAKAK (SEQ ID NO: 48), RADARADARADARADA (SEQ ID NO: 50), RADARGDARADARGDA (SEQ ID NO: 76), RADARADA (SEQ ID NO: 77), RARADADARARADADA (SEQ ID NO: 51), RARADADA (SEQ ID NO: 78), RARARARADADADADA (SEQ ID NO: 79), ADADADADARARARAR (SEQ ID NO: 80), DADADADARARARARA (SEQ ID NO: 81), RAEARAEARAEARAEA (SEQ ID NO: 82), RAEARAEA (SEQ ID NO: 83), KAKAKAKAEAEAEAEA (SEQ ID NO: 84), AEAEAEAEAKAKAKAK (SEQ ID NO: 49), KADAKADAKADAKADA (SEQ ID NO: 85), KADAKADA (SEQ ID NO: 86), AEAEAHAHAEAEAHAHA (SEQ ID NO: 87), AEAEAHAHA (SEQ ID NO: 88), HEHEHKHKHEHEHKHK (SEQ ID NO: 89), HEHEHKHK (SEQ ID NO: 90), FEFEFKFKFEFEFKFK (SEQ ID NO: 91), FEFKFEFK (SEQ ID NO: 92), LELELKLKLELELKLK (SEQ ID NO: 93), LELELKLK (SEQ ID NO: 94), KFDLKKDLKLDL (SEQ ID NO: 95), FKFEFKFF (SEQ ID NO: 96), FEFEFKFK (SEQ ID NO: 97), and RFRFRFRFRFRFRFRFRFRF (SEQ ID NO: 98); and (ii) at least one PC molecule conjugated to a terminus of the self-assembling peptide.

Clause 45. The PC-peptide conjugate of clause 44, wherein a plurality of the PC-peptide conjugates assembles into a nanofiber, nanotube, hydrogel, micelle, vesicle, nanoparticle, or suspension.

Clause 46. A pharmaceutical composition comprising: (a) the PC-conjugate peptide of any one of clauses 1-23 or the nanofiber of any one of clauses 24-45; and (b) a pharmaceutically acceptable carrier, diluent, and/or excipient.

Clause 47. A method of stimulating B1a cells to express antibodies, the method comprising contacting at least one B1a cell with the PC-conjugate peptide of any one of clauses 1-23 or the nanofiber of any one of clauses 24-45, such that the B1a cells in the subject express antibodies.

Clause 48. A method of producing anti-PC antibodies in a subject, the method comprising administering to the subject an effective amount of the PC-conjugate peptide of any one of clauses 1-23 or the nanofiber of any one of clauses 24-45 or the pharmaceutical composition of clause 46, such that anti-PC antibodies are produced in the subject.

Clause 49. The method of clause 47 or 48, wherein the antibodies are selected from IgM, IgG, and IgA, or a combination thereof.

Clause 50. A method of treating an inflammatory disease or disorder in a subject, the method comprising administering to the subject a therapeutically effective amount of the PC-conjugate peptide of any one of clauses 1-23 or the nanofiber of any one of clauses 24-45 or the pharmaceutical composition of clause 46.

Clause 51. The method of clause 50, wherein the inflammatory disease or disorder comprises inflammatory bowel disease (IBD), ulcerative colitis, Crohn's disease, rheumatoid arthritis, psoriasis, cirrhosis, atherosclerosis, cardiovascular inflammation, or a combination thereof.

Clause 52. A method of altering gut microbiome activity in a subject, the method comprising administering to the subject a therapeutically effective amount of the conjugate peptide of any one of clauses 1-23 or the nanofiber of any one of clauses 24-45 or the pharmaceutical composition of clause 46.

Clause 53. The method of any one of clauses 47-52, wherein the bacterial diversity in the colon is reduced.

Clause 54. The method of any one of clauses 47-53, wherein the amount of viable bacteria spread to the spleen from the colon is reduced.

Clause 55. The method of any one of clauses 47-54, wherein the PC-peptide conjugate or the nanofiber is administered to the subject intraperitoneally, orally, sublingually, intravenously, nasally, buccally, transdermally, intranasally, or intramuscularly, or via the lung.

Clause 56. The method of any one of clauses 47-55, the method further comprising administering at least one additional therapeutic agent.

Clause 57. The method according to clause 56, wherein the at least one additional therapeutic agent is administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition.

Clause 58. The method of any one of clauses 56-57, wherein the at least one additional therapeutic agent comprises an adjuvant.

Clause 59. The method of clause 58, wherein the adjuvant comprises CpG, or cholera toxin B subunit (CTB), or STING agonist, or cyclic dinucleotides, or alum, or MF59, or a combination thereof.

What is claimed is:

1. A phosphorylcholine (PC)-conjugate peptide comprising:
(i) a self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and
(ii) at least one PC molecule conjugated to a terminus of the self-assembling peptide.

2. The PC-conjugate peptide of claim 1, wherein each self-assembling peptide forms a beta sheet and comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn).

3. The PC-conjugate peptide according to claim 1 or 2, wherein the self-assembling peptide comprises the sequence QQKFQFQFEQQ (SEQ ID NO: 31) or Ac-QQKFQFQFEQQ-NH₂ (SEQ ID NO: 99).

4. The PC-conjugate peptide of claim 1, wherein each self-assembling peptide forms an alpha-helix and comprises a polypeptide having an amino acid sequence of bXXXb (SEQ ID NO: 1), wherein X is independently any amino acid and b is independently any positively charged amino acid.

5. The PC-conjugate peptide of claim 1 or 4, wherein b is independently selected from Arg and Lys.

6. The PC-conjugate peptide of claim 1, 4, or 5, wherein bXXXb (SEQ ID NO: 1) is RAYAR (SEQ ID NO: 2) or KAYAK (SEQ ID NO: 3).

7. The PC-conjugate peptide of any one of claims 1 and 4-6, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

8. The PC-conjugate peptide of claim 7, wherein the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 22), or Ac-QAKILEADAEILKAYAKILEAHAEILKAQ-NH₂ (SEQ ID NO: 23), or Ac-ADAEILRAYARILEAHAEILRAQ-NH₂ (SEQ ID NO: 24).

9. The PC-conjugate peptide of any one of claims 1-8, further comprising:
(iii) a linker between the at least one PC molecule and the self-assembling peptide.

10. The PC-conjugate peptide of claim 9, wherein the linker comprises an amino acid sequence selected from SEQ ID NO: 25 ((Ser-Gly)₂), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), SEQ ID NO: 29 (EAAAK)₂, and SEQ ID NO: 30 (GGAAY).

11. The PC-conjugate peptide of claim 10, wherein the linker comprises (Ser-Gly)₂ (SEQ ID NO: 25).

12. The PC-conjugate peptide of any one of claims 9-11, wherein each PC molecule is attached to the linker via a Cys residue or an Asn residue.

13. The PC-conjugate peptide of any one of claims 1-12, wherein the at least one PC molecule is attached to the C-terminus or the N-terminus of the self-assembling peptide.

14. The PC-conjugate peptide of any one of claims 1-13, wherein 1 to 10 PC molecules are attached to the C-terminus or the N-terminus of the self-assembling peptide.

15. The PC-conjugate peptide of claim 14, wherein each PC molecule is independently attached to the linker via a Cys residue or an Asn residue.

16. The PC-conjugate peptide of any one of claims 1-15, wherein 1 to 10 PC molecules are attached to the C-terminus or the N-terminus of the self-assembling peptide via a single linker.

17. The PC-conjugate peptide of claim 16, wherein 1 to 4 PC molecules are attached to the C-terminus or the N-terminus of the self-assembling peptide.

18. The PC-conjugate peptide of claim 17, wherein 4 PC molecules are attached to the C-terminus or the N-terminus of the self-assembling peptide.

19. The PC-conjugate peptide of claim 18, comprising the sequence of (PC molecule)₄-CCCCSGSG-QQKFQFQFEQQ-NH₂ SEQ ID NO: 34), wherein each one of the four PC molecules is attached to a different Cys in the linker CCCCSGSG (SEQ ID NO: 26).

20. The PC-conjugate peptide of any one of claims 1-19, wherein each PC molecule is independently selected from C₅H₁₄NO₄P and C₁₁H₂₂NO₆P.

21. The PC-conjugate peptide of any one of claims 1-20, wherein the PC-conjugate peptide comprises a sequence selected from C₅H₁₄NO₄P-SGSG-QQKFQFQFEQQ-NH₂ (pa-PC-Q11, SEQ ID NO: 32), or C₁₁H₂₂NO₆P-CSGSG-QQKFQFQFEQQ-NH₂ (PC-Q11, SEQ ID NO: 33), or (C₁₁H₂₂NO₆P)ₙ-CCCCSGSG-QQKFQFQFEQQ-NH₂ (PC_{M}-Q11, SEQ ID NO: 34) wherein n is an integer selected from 1, 2, 3, and 4).

22. The PC-conjugate peptide of any one of claims 1-21, further comprising a PAS peptide of SEQ ID NO: 35 or 36 conjugated to the self-assembling peptide at the opposite terminus from wherein the PC molecule is attached.

23. A nanofiber comprising a plurality of the PC-conjugate peptide of any one of claims 1-22, wherein the conjugate peptide self-assembles into the nanofiber.

24. A nanofiber comprising:
(i) at least one PC-conjugate peptide of any one of claims 1-22; and
(ii) at least one PADRE-conjugate peptide comprising:
a self-assembling peptide, wherein each self-assembling peptide independently comprises a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid); and
at least one PADRE molecule, wherein at least one self-assembling peptide is conjugated to at least one PADRE molecule, and wherein the PADRE molecule comprises a polypeptide having the amino acid sequence of aKXVAAWTLKAa (SEQ ID NO: 18, wherein “X” comprises cyclohexylalanine and “a” comprises D-alanine).

25. The nanofiber of claim 24, wherein the PADRE-conjugate peptide further comprises a linker between the PADRE molecule and the self-assembling peptide.

26. The nanofiber of claim 25, wherein the linker comprises an amino acid sequence selected from SEQ ID NO: 25 ((Ser-Gly)₂), SEQ ID NO: 26 (CCCCSGSG), SEQ ID NO: 9 (Gₙ wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 27 (KSGSG), SEQ ID NO: 28 (KKSGSG), and SEQ ID NO: 29 (EAAAK)₂, and SEQ ID NO: 30 (GGAAY).

27. The nanofiber of any one of claims 24-26, wherein the at least one PADRE-conjugate peptide comprises the sequence of NH₂-aKXVAAWTLKAa-SGSG-QQKFQFQFEQQ-NH₂ (PADRE-Q11, SEQ ID NO: 37).

28. The nanofiber of any one of claims 24-27, wherein the cyclohexylalanine comprises D-alanine.

29. The nanofiber of any one of claims 24-28, further comprising:
(iv) a plain self-assembling peptide comprising a polypeptide having an amino acid sequence selected from QQKFQFQFEQQ (SEQ ID NO: 31), FKFEFKFE (SEQ ID NO: 38), KFQFQFE (SEQ ID NO: 39), QQRFQFQFEQQ (SEQ ID NO: 40), QQRFQWQFEQQ (SEQ ID NO: 41), FEFEFKFKFEFEFKFK (SEQ ID NO: 42), QQRFEWEFEQQ (SEQ ID NO: 43), QQXFXWXFQQQ (SEQ ID NO: 44, where X is ornithine), FKFEFKFEFKFE (SEQ ID NO: 45), FKFQFKFQFKFQ (SEQ ID NO: 46), AEAKAEAKAEAKAEAK (SEQ ID NO: 47), AEAEAKAKAEAEAKAK (SEQ ID NO: 48), AEAEAEAEAKAKAKAK (SEQ ID NO: 49), RADARADARADARADA (SEQ ID NO: 50), RARADADARARADADA (SEQ ID NO: 51), SGRGYBLGGQGAGAAAAAGGAGQGGYGGLGSQG (SEQ ID NO: 52), EWEXEXEXEX (SEQ ID NO: 53, where X is Val, Ala, Ser, or Pro), WKXKXKXKXK (SEQ ID NO:54, where X is Val, Ala, Ser, or Pro), KWKVKVKVKVKVKVK (SEQ ID NO: 55, where X is Val, A, Ser, or Pro), LLLLKKKKKKKKLLLL (SEQ ID NO: 56), VKVKVKVKVDPPTKVKVKVKV (SEQ ID NO: 57), VKVKVKVKVDPPTKVKTKVKV (SEQ ID NO: 58), KVKVKVKVKDPPSVKVKVKVK (SEQ ID NO: 59), VKVKVKVKVDPPSKVKVKVKV (SEQ ID NO: 60), VKVKVKTKVDPPTKVKTKVKV (SEQ ID NO: 61), QQKFxFQFEQQ (SEQ ID NO: 62, wherein x is Glu, Asp, or Asn), QQKFQxQFEQQ (SEQ ID NO: 63, wherein x is Trp or Tyr), and QQKFQFxFEQQ (SEQ ID NO: 64, wherein x is Glu, Asp, or Asn), and bXXXb (SEQ ID NO: 1, wherein X is independently any amino acid and b is independently any positively charged amino acid), without any PC molecules attached thereto.

30. The nanofiber of any one of claims 24-29, wherein the nanofiber comprises a combination of 10 to 10,000, or 100 to 10,000 peptides comprising PC-conjugate peptides and PADRE-conjugate peptides.

31. The nanofiber of claim 29, wherein the nanofiber comprises a combination of 10 to 10,000, or 100 to 10,000 peptides comprising PC-conjugate peptides, PADRE-conjugate peptides, and plain self-assembling peptides.

32. The nanofiber of any one of claims 24-31, wherein at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are PC-conjugate peptides.

33. The nanofiber of any one of claims 24-32, wherein at least about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the peptides in the nanofiber are PADRE-conjugate peptides.

34. The nanofiber of any one of claims 29-33, wherein at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides.

35. The nanofiber of any one of claims 29-34, wherein about 50% of the peptides in the nanofiber are PC-conjugate peptides, about 2.5% of the peptides are PADRE-conjugate peptides, and about 47.5% of the peptides are plain self-assembling peptides.

36. The nanofiber of any one of claims 24-35, wherein the PC-peptide conjugate and the PADRE-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

37. The nanofiber of any one of claims 24-36, wherein the self-assembling peptide forms a fibril including beta-sheet structures.

38. The nanofiber of any one of claims 24-36, wherein the self-assembling peptide forms a fibril having a coiled coil structure.

39. The nanofiber of any one of claims 24-36 and 38, wherein the self-assembling peptide forms a fibril having a structure of a helical filament formed around a central axis.

40. The nanofiber of claim 39, wherein the N-terminus of each self-assembling peptide is positioned at the exterior of the helical filament.

41. The nanofiber of any one of claims 24-40, wherein the PC molecules are exposed on the exterior surface of the nanofiber.

42. The nanofiber of any one of claims 24-41, wherein the nanofiber is about 5-20 nm in width.

43. The nanofiber of any one of claims 24-42, wherein the nanofiber is about 100 nm to 1 μm, 100 nm to 2 μm, 100 nm to 3 μm, 100 nm to 4 μm, or 100 nm to 5 μm in length.

44. A phosphorylcholine (PC)-conjugate peptide comprising:
(i) a self-assembling peptide, wherein the self-assembling peptide comprises a polypeptide having an amino acid sequence selected from VEVKVEVKV (SEQ ID NO: 65), VEVKVEVKVEVK (SEQ ID NO: 66), VVVAAAEEE (SEQ ID NO: 67), VEVEVEVEVEVEVEVEVEVE (SEQ ID NO: 68), CGNKRTRGC (SEQ ID NO: 69), VKVKVKVKVDPPTKVEVKVKV (SEQ ID NO: 70), LRKKLGKA (SEQ ID NO: 71), VVVVVVKK (SEQ ID NO: 72), AEAKAEAKAEAKAEAK (SEQ ID NO: 47), AEAKAEAK (SEQ ID NO: 73), AEAEAEAEAKAK (SEQ ID NO: 74), AEAEAKAK (SEQ ID NO: 75), AEAEAKAKAEAEAKAK (SEQ ID NO: 48), RADARADARADARADA (SEQ ID NO: 50), RADARGDARADARGDA (SEQ ID NO: 76), RADARADA (SEQ ID NO: 77), RARADADARARADADA (SEQ ID NO: 51), RARADADA (SEQ ID NO: 78), RARARARADADADADA (SEQ ID NO: 79), ADADADADARARARAR (SEQ ID NO: 80), DADADADARARARARA (SEQ ID NO: 81), RAEARAEARAEARAEA (SEQ ID NO: 82), RAEARAEA (SEQ ID NO: 83), KAKAKAKAEAEAEAEA (SEQ ID NO: 84), AEAEAEAEAKAKAKAK (SEQ ID NO: 49), KADAKADAKADAKADA (SEQ ID NO: 85), KADAKADA (SEQ ID NO: 86), AEAEAHAHAEAEAHAHA (SEQ ID NO: 87), AEAEAHAHA (SEQ ID NO: 88), HEHEHKHKHEHEHKHK (SEQ ID NO: 89), HEHEHKHK (SEQ ID NO: 90), FEFEFKFKFEFEFKFK (SEQ ID NO: 91), FEFKFEFK (SEQ ID NO: 92), LELELKLKLELELKLK (SEQ ID NO: 93), LELELKLK (SEQ ID NO: 94), KFDLKKDLKLDL (SEQ ID NO: 95), FKFEFKFF (SEQ ID NO: 96), FEFEFKFK (SEQ ID NO: 97), and RFRFRFRFRFRFRFRFRFRF (SEQ ID NO: 98); and
(ii) at least one PC molecule conjugated to a terminus of the self-assembling peptide.

45. The PC-peptide conjugate of claim 44, wherein a plurality of the PC-peptide conjugates assembles into a nanofiber, nanotube, hydrogel, micelle, vesicle, nanoparticle, or suspension.

46. A pharmaceutical composition comprising: (a) the PC-conjugate peptide of any one of claims 1-23 or the nanofiber of any one of claims 24-45; and (b) a pharmaceutically acceptable carrier, diluent, and/or excipient.

47. A method of stimulating B1a cells to express antibodies, the method comprising contacting at least one B1a cell with the PC-conjugate peptide of any one of claims 1-23 or the nanofiber of any one of claims 24-45, such that the B1a cells in the subject express antibodies.

48. A method of producing anti-PC antibodies in a subject, the method comprising administering to the subject an effective amount of the PC-conjugate peptide of any one of claims 1-23 or the nanofiber of any one of claims 24-45 or the pharmaceutical composition of claim 46, such that anti-PC antibodies are produced in the subject.

49. The method of claim 47 or 48, wherein the antibodies are selected from IgM, IgG, and IgA, or a combination thereof.

50. A method of treating an inflammatory disease or disorder in a subject, the method comprising administering to the subject a therapeutically effective amount of the PC-conjugate peptide of any one of claims 1-23 or the nanofiber of any one of claims 24-45 or the pharmaceutical composition of claim 46.

51. The method of claim 50, wherein the inflammatory disease or disorder comprises inflammatory bowel disease (IBD), ulcerative colitis, Crohn's disease, rheumatoid arthritis, psoriasis, cirrhosis, atherosclerosis, cardiovascular inflammation, or a combination thereof.

52. A method of altering gut microbiome activity in a subject, the method comprising administering to the subject a therapeutically effective amount of the conjugate peptide of any one of claims 1-23 or the nanofiber of any one of claims 24-45 or the pharmaceutical composition of claim 46.

53. The method of any one of claims 47-52, wherein the bacterial diversity in the colon is reduced.

54. The method of any one of claims 47-53, wherein the amount of viable bacteria spread to the spleen from the colon is reduced.

55. The method of any one of claims 47-54, wherein the PC-peptide conjugate or the nanofiber is administered to the subject intraperitoneally, orally, sublingually, intravenously, nasally, buccally, transdermally, intranasally, or intramuscularly, or via the lung.

56. The method of any one of claims 47-55, the method further comprising administering at least one additional therapeutic agent.

57. The method according to claim 56, wherein the at least one additional therapeutic agent is administered prior to the PC-conjugate peptide or the nanofiber or the pharmaceutical composition, or concurrently with the PC-conjugate peptide or the nanofiber or the pharmaceutical composition, or after the PC-conjugate peptide or the nanofiber or the pharmaceutical composition.

58. The method of any one of claims 56-57, wherein the at least one additional therapeutic agent comprises an adjuvant.

59. The method of claim 58, wherein the adjuvant comprises CpG, or cholera toxin B subunit (CTB), or STING agonist, or cyclic dinucleotides, or alum, or MF59, or a combination thereof.


SEQUENCE LISTING (100 sequences)

SEQ ID NO: 1 - skipped

SEQ ID NO: 2 - protein, 5 aa, synthetic construct
RAYAR

SEQ ID NO: 3 - protein, 5 aa, synthetic construct
KAYAK

SEQ ID NO: 4 - skipped

SEQ ID NO: 5 - protein, 7 aa, synthetic construct
  VARIANT 1: note = independently any amino acid
  REPEAT 1: note = repeat from 0 to 20
  VARIANT 2: note = independently any positively charged amino acid
  VARIANT 3..5: note = independently any amino acid
  VARIANT 6: note = independently any positively charged amino acid
  VARIANT 7: note = independently any amino acid
  REPEAT 7: note = repeat 0 to 20
ZBXXXBZ

SEQ ID NO: 6 - protein, 29 aa, synthetic construct
QARILEADAEILRAYARILEAHAEILRAQ

SEQ ID NO: 7 - protein, 29 aa, synthetic construct
QAKILEADAEILKAYAKILEAHAEILKAQ

SEQ ID NO: 8 - protein, 23 aa, synthetic construct
ADAEILRAYARILEAHAEILRAQ

SEQ ID NO: 9 - protein, 10 aa, synthetic construct
  VARIANT 2..10: note = may or may not be present
GGGGGGGGGG

SEQ ID NO: 10 - protein, 4 aa, synthetic construct
SGSG

SEQ ID NO: 11 - protein, 4 aa, synthetic construct
GSGS

SEQ ID NO: 12 - protein, 4 aa, synthetic construct
SSSS

SEQ ID NO: 13 - protein, 4 aa, synthetic construct
GGGS

SEQ ID NO: 14 - skipped

SEQ ID NO: 15 - protein, 24 aa, synthetic construct
GGCGGCGGCGGCGGCGGCGGCGGC

SEQ ID NO: 16 - protein, 15 aa, synthetic construct
GGGGSGGGGSGGGGS

SEQ ID NO: 17 - protein, 13 aa, synthetic construct
LEEKKGNYVVTDH

SEQ ID NO: 18 - protein, 12 aa, synthetic construct
  SITE 1: note = D-alanine
  SITE 3: note = cyclohexylalanine
  SITE 12: note = D-alanine
XKXVAAWTLKAX

SEQ ID NO: 19 - protein, 33 aa, synthetic construct
SGSGQARILEADAEILRAYARILEAHAEILRAQ

SEQ ID NO: 20 - protein, 46 aa, synthetic construct
LEEKKGNYVVTDHSGSGQARILEADAEILRAYARILEAHAEILRAQ

SEQ ID NO: 21 - protein, 45 aa, synthetic construct
  SITE 1: note = D-alanine
  SITE 3: note = cyclohexylalanine
  SITE 12: note = D-alanine
AKXVAAWTLKAASGSGQARILEADAEILRAYARILEAHAEILRAQ

SEQ ID NO: 22 - protein, 29 aa, synthetic construct
  MOD_RES <1: note = N-terminal acetylation
  MOD_RES >29: note = C-terminal amidation
QARILEADAEILRAYARILEAHAEILRAQ

SEQ ID NO: 23 - protein, 29 aa, synthetic construct
  MOD_RES <1: note = N-terminal acetylation
  MOD_RES >29: note = C-terminal amidation
QAKILEADAEILKAYAKILEAHAEILKAQ

SEQ ID NO: 24 - protein, 23 aa, synthetic construct
  MOD_RES <1: note = N-terminal acetylation
  MOD_RES >23: note = C-terminal amidation
ADAEILRAYARILEAHAEILRAQ

SEQ ID NO: 25 - protein, 4 aa, synthetic construct
SGSG

SEQ ID NO: 26 - protein, 8 aa, synthetic construct
CCCCSGSG

SEQ ID NO: 27 - protein, 5 aa, synthetic construct
KSGSG

SEQ ID NO: 28 - protein, 6 aa, synthetic construct
KKSGSG

SEQ ID NO: 29 - protein, 10 aa, synthetic construct
EAAAKEAAAK

SEQ ID NO: 30 - protein, 5 aa, synthetic construct
GGAAY

SEQ ID NO: 31 - protein, 11 aa, synthetic construct
QQKFQFQFEQQ

SEQ ID NO: 32 - protein, 15 aa, synthetic construct
SGSGQQKFQFQFEQQ

SEQ ID NO: 33 - protein, 16 aa, synthetic construct
CSGSGQQKFQFQFEQQ

SEQ ID NO: 34 - protein, 19 aa, synthetic construct
CCCCSGSGQQKFQFQFEQQ

SEQ ID NO: 35 - protein, 20 aa, synthetic construct
ASPAAPAPASPAAPAPSAPA

SEQ ID NO: 36 - protein, 20 aa, synthetic construct
ASPAAPAPASPAAPAPSAPA

SEQ ID NO: 37 - protein, 27 aa, synthetic construct
  SITE 1: note = D-alanine
  SITE 3: note = cyclohexylalanine
  SITE 12: note = D-alanine
AKXVAAWTLKAASGSGQQKFQFQFEQQ

SEQ ID NO: 38 - protein, 8 aa, synthetic construct
FKFEFKFE

SEQ ID NO: 39 - protein, 7 aa, synthetic construct
KFQFQFE

SEQ ID NO: 40 - protein, 11 aa, synthetic construct
QQRFQFQFEQQ

SEQ ID NO: 41 - protein, 11 aa, synthetic construct
QQRFQWQFEQQ

SEQ ID NO: 42 - protein, 16 aa, synthetic construct
FEFEFKFKFEFEFKFK

SEQ ID NO: 43 - protein, 11 aa, synthetic construct
QQRFEWEFEQQ

SEQ ID NO: 44 - protein, 11 aa, synthetic construct
  SITE 3: note = ornithine
  SITE 5: note = ornithine
  SITE 7: note = ornithine
QQXFXWXFQQQ

SEQ ID NO: 45 - protein, 12 aa, synthetic construct
FKFEFKFEFKFE

SEQ ID NO: 46 - protein, 12 aa, synthetic construct
FKFQFKFQFKFQ

SEQ ID NO: 47 - protein, 16 aa, synthetic construct
AEAKAEAKAEAKAEAK

SEQ ID NO: 48 - protein, 16 aa, synthetic construct
AEAEAKAKAEAEAKAK

SEQ ID NO: 49 - protein, 16 aa, synthetic construct
AEAEAEAEAKAKAKAK

SEQ ID NO: 50 - protein, 16 aa, synthetic construct
RADARADARADARADA

SEQ ID NO: 51 - protein, 16 aa, synthetic construct
RARADADARARADADA

SEQ ID NO: 52 - protein, 33 aa, synthetic construct
SGRGYBLGGQGAGAAAAAGGAGQGGYGGLGSQG

SEQ ID NO: 53 - protein, 10 aa, synthetic construct
  VARIANT 4: note = Val, Ala, Ser, or Pro
  VARIANT 6: note = Val, Ala, Ser, or Pro
  VARIANT 8: note = Val, Ala, Ser, or Pro
  VARIANT 10: note = Val, Ala, Ser, or Pro
EWEXEXEXEX

SEQ ID NO: 54 - protein, 10 aa, synthetic construct
  VARIANT 3: note = Val, Ala, Ser, or Pro
  VARIANT 5: note = Val, Ala, Ser, or Pro
  VARIANT 7: note = Val, Ala, Ser, or Pro
  VARIANT 9: note = Val, Ala, Ser, or Pro
WKXKXKXKXK

SEQ ID NO: 55 - protein, 15 aa, synthetic construct
KWKVKVKVKVKVKVK

SEQ ID NO: 56 - protein, 16 aa, synthetic construct
LLLLKKKKKKKKLLLL

SEQ ID NO: 57 - protein, 21 aa, synthetic construct
VKVKVKVKVDPPTKVKVKVKV

SEQ ID NO: 58 - protein, 21 aa, synthetic construct
VKVKVKVKVDPPTKVKTKVKV

SEQ ID NO: 59 - protein, 21 aa, synthetic construct
KVKVKVKVKDPPSVKVKVKVK

SEQ ID NO: 60 - protein, 21 aa, synthetic construct
VKVKVKVKVDPPSKVKVKVKV

SEQ ID NO: 61 - protein, 21 aa, synthetic construct
VKVKVKTKVDPPTKVKTKVKV

SEQ ID NO: 62 - protein, 11 aa, synthetic construct
  VARIANT 5: note = Glu, Asp, or Asn
QQKFXFQFEQQ

SEQ ID NO: 63 - protein, 11 aa, synthetic construct
  VARIANT 6: note = Trp or Tyr
QQKFQXQFEQQ

SEQ ID NO: 64 - protein, 11 aa, synthetic construct
  VARIANT 7: note = Glu, Asp, or Asn
QQKFQFXFEQQ

SEQ ID NO: 65 - protein, 9 aa, synthetic construct
VEVKVEVKV

SEQ ID NO: 66 - protein, 12 aa, synthetic construct
VEVKVEVKVEVK

SEQ ID NO: 67 - protein, 9 aa, synthetic construct
VVVAAAEEE

SEQ ID NO: 68 - protein, 20 aa, synthetic construct
VEVEVEVEVEVEVEVEVEVE

SEQ ID NO: 69 - protein, 9 aa, synthetic construct
CGNKRTRGC

SEQ ID NO: 70 - protein, 21 aa, synthetic construct
VKVKVKVKVDPPTKVEVKVKV

SEQ ID NO: 71 - protein, 8 aa, synthetic construct
LRKKLGKA

SEQ ID NO: 72 - protein, 8 aa, synthetic construct
VVVVVVKK

SEQ ID NO: 73 - protein, 8 aa, synthetic construct
AEAKAEAK

SEQ ID NO: 74 - protein, 12 aa, synthetic construct
AEAEAEAEAKAK

SEQ ID NO: 75 - protein, 8 aa, synthetic construct
AEAEAKAK

SEQ ID NO: 76 - protein, 16 aa, synthetic construct
RADARGDARADARGDA

SEQ ID NO: 77 - protein, 8 aa, synthetic construct
RADARADA

SEQ ID NO: 78 - protein, 8 aa, synthetic construct
RARADADA

SEQ ID NO: 79 - protein, 16 aa, synthetic construct
RARARARADADADADA

SEQ ID NO: 80 - protein, 16 aa, synthetic construct
ADADADADARARARAR

SEQ ID NO: 81 - protein, 16 aa, synthetic construct
DADADADARARARARA

SEQ ID NO: 82 - protein, 16 aa, synthetic construct
RAEARAEARAEARAEA

SEQ ID NO: 83 - protein, 8 aa, synthetic construct
RAEARAEA

SEQ ID NO: 84 - protein, 16 aa, synthetic construct
KAKAKAKAEAEAEAEA

SEQ ID NO: 85 - protein, 16 aa, synthetic construct
KADAKADAKADAKADA

SEQ ID NO: 86 - protein, 8 aa, synthetic construct
KADAKADA

SEQ ID NO: 87 - protein, 17 aa, synthetic construct
AEAEAHAHAEAEAHAHA

SEQ ID NO: 88 - protein, 9 aa, synthetic construct
AEAEAHAHA

SEQ ID NO: 89 - protein, 16 aa, synthetic construct
HEHEHKHKHEHEHKHK

SEQ ID NO: 90 - protein, 8 aa, synthetic construct
HEHEHKHK

SEQ ID NO: 91 - protein, 16 aa, synthetic construct
FEFEFKFKFEFEFKFK

SEQ ID NO: 92 - protein, 8 aa, synthetic construct
FEFKFEFK

SEQ ID NO: 93 - protein, 16 aa, synthetic construct
LELELKLKLELELKLK

SEQ ID NO: 94 - protein, 8 aa, synthetic construct
LELELKLK

SEQ ID NO: 95 - protein, 12 aa, synthetic construct
KFDLKKDLKLDL

SEQ ID NO: 96 - protein, 8 aa, synthetic construct
FKFEFKFF

SEQ ID NO: 97 - protein, 8 aa, synthetic construct
FEFEFKFK

SEQ ID NO: 98 - protein, 20 aa, synthetic construct
RFRFRFRFRFRFRFRFRFRF

SEQ ID NO: 99 - protein, 11 aa, synthetic construct
  MOD_RES <1: note = N-terminal acetylation
  MOD_RES >11: note = C-terminal amidation
QQKFQFQFEQQ

SEQ ID NO: 100 - protein, 16 aa, synthetic construct
NSGSGQQKFQFQFEQQ